(19) United States
(12) Patent Application Publication
(10) Pub. No.: US 2025/0326699 A1
(43) Pub. Date: Oct. 23, 2025

(54) USE OF A DILUENT TO MICROBIAL FERTILIZER FOR INCREASED EFFICACY AND/OR SHELF LIFE

(71) Applicant: KULA BIO, INC., Natick, MA (US)

(72) Inventors: Sung-Yong Harrison YOON, Natick, MA (US); Frederic KENDIRGI, Fort Collins, CO (US); Kyle CARTER, Natick, MA (US); Darian SMERCINA, Pasco, WA (US)

(21) Appl. No.: 19/183,150

(22) Filed: Apr. 18, 2025

Related U.S. Application Data
(60) Provisional application No. 63/636,506, filed on Apr. 19, 2024.

(51) Int. Cl. C05F 11/08; C05F 1/00; C05F 9/00; C12N 1/14; C12N 1/20; C12R 1/39

(52) U.S. Cl. CPC C05F 11/08; C05F 1/00; C05F 9/00; C12N 1/14; C12N 1/20; C12R 2001/39

(57) ABSTRACT

The disclosure provides a composition comprising i) a diluent derived from a plant, yeast, insect, crustacean or algae, and ii) a nitrogen fixing microorganism; along with methods of preparing and using said composition.

### CROSS REFERENCE TO RELATED APPLICATIONS

The present application claims the priority benefit of U.S. Provisional Application No. 63/636,506, filed Apr. 19, 2024, which is hereby incorporated by reference in its entirety.

### FIELD OF THE DISCLOSURE

The disclosure relates to biofertilizers and diluents extracted from plants, yeast, insects, crustaceans or algae.

### BACKGROUND OF THE DISCLOSURE

Microbial-based fertilizers or biostimulants are produced through fermentation processes to generate single or multi-strain products that can be applied in conjunction with standard farmer practices. The focus of fermentation is to achieve a total cell and/or spore density that provides the best plant responses such as increase biomass, fruit quality and/or yield, growth disease and/or chemical over application resistance. Traditionally the product of the fermentation process is applied directly with minimal downstream processing at a given rate based on trials and experimentation.

However, a need exists for new microbial-based fertilizer products.

### BRIEF SUMMARY

Certain aspects of the disclosure provide a composition comprising i) a diluent and ii) a microorganism. In some aspects, the diluent is derived from a plant, yeast, insect, crustacean or algae. In some aspects, the microorganism is a nitrogen fixing microorganism.

In some aspects, the diluent is in a dry formulation.

In some aspects, the nitrogen fixing microorganism is in a dry formulation.

In some aspects, the diluent is in a liquid formulation.

In some aspects, the nitrogen fixing microorganism is in a liquid formulation.

In some aspects, the composition further comprises one, two, three, four, five, six, or more nitrogen fixing or non-nitrogen fixing microorganisms.

In some aspects, the composition comprises Xanthobacter autotrophicus, Pseudomonas fluorescens, Rhodopseudomonas palustris, Azospirillum lipoferum, Cupriavidus necator, or combinations thereof.

In some aspects, the composition comprises Xanthobacter autotrophicus and Pseudomonas fluorescens.

In some aspects, the composition comprises Xanthobacter autotrophicus and Rhodopseudomonas palustris.

In some aspects, the composition comprises Xanthobacter autotrophicus and Azospirillum lipoferum.

In some aspects, composition comprises Xanthobacter autotrophicus and Cupriavidus necator.

In some aspects, the diluent is derived from a plant.

In some aspects, the plant is selected from a group consisting of a coffee plant, a carrot, a potato, a citrus plant, a banana, an alfalfa grass, a tomato, a grape, a rice, a maple tree, and combinations thereof.

In some aspects, the diluent is derived from a peel of a banana, a rind of a citrus plant, a coffee ground, waste from a grape, a husk of a rice plant, or a maple syrup from a maple tree.

In some aspects, the diluent is derived from an algae.

In some aspects, the diluent is derived from a yeast.

In some aspects, the yeast is a brewing yeast.

In some aspects, the yeast is a spent brewing yeast.

In some aspects, the diluent is derived from an insect.

In some aspects, the insect is a bee.

In some aspects, the diluent is derived from honey or bee's wax.

In some aspects, the nitrogen fixing microorganism is a bacteria.

In some aspects, the bacteria is selected from the group consisting of Acidiphilium multivorum, Acidiphilium species, Alcaligenes paradoxus, Alcaligenes species, Arthrobacter species, Azoarcus indigens, Azohydromonas australica, Azohydromonas lata, Azohydromonas species, Azorhizobium caulinodans, Azospirillium brasiliense, Azospirillium spp., Azospirillum amazonsense, Azospirillum lipoferum, Azospirillum lipoferum (RSAL0111), Azospirillum species, Azospirillum thiophilum, Azotobacter chroococum (MCC 0055), Azotobacter spp., Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beggiatoa species, Beijerinckia mobilis, Beijerinckia species, Bradyrhizobium elnakii, Bradyrhizobium japonicum, Bradyrhizobium japonicum (strain USDA 122), Bradyrhizobium species, Burkholderia species, Burkholderia vietnameiensis, Cupriavidus necator, Cupriavidus species, Cyanobacteria spp., Derxia gummosa, Derxia species, Gluconacetobacter diazotrophicus, Gluconacetobacter diazotrophicus (MCC 0046), Herbaspirillum autrotrophicum, Herbaspirillum frisingense (MCC 0052), Herbaspirillum species, Hydrogenophaga pseudoflava, Hydrogenophaga species, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Mesorhizobium species, Methylibium petroleiphilum, Methylibium species, Methylocapsa aurea, Methylocapsa species, Methyloferula species, Methyloferula stellate, Methyloversatilis species, Methyloversatilis universalis, Microcyclus aquaticus, Microcyclus ebruneus, Microcyclus species, Nitrosococcus oceani, Nitrosococcus species, Nitrosomonas communis, Nitrospirillum amazonense, Nocardia autotrophica, Nocardia opaca, Nocardia species, Oligotropha carboxidovorans, Oligotropha species, Paenibacillus durus (MCC 0046), Pannonibacter phragmitetus, Pannonibacter species, Paracoccus denitrificans, Paracoccus pantrophus, Paracoccus species, Paracoccus yeei, Pelagibaca bermudensis, Pelagibaca species, Pseudomonas facilis, Pseudomonas fluorescens, Pseudomonas species, Pseudooceanicola atlanticus, Pseudooceanicola species, Ralstonia eutropha, Ralstonia species, Renobacter species, Renobacter vacuolatum, Rhizobium gallicum, Rhizobium japonicum, Rhizobium japonicum (MCC 0071), Rhizobium leguminosarum, Rhizobium leguminosarum biovar viciae, Rhizobium species, Rhizobium spp., Rhodobacter capsulatus, Rhodobacter species, Rhodobacter sphaeroides, Rhodomicrobium species, Rhodomicrobium vannielii, Rhodopseudomonas palustris, Rubrivivax gelatinosus, Rubrivivax species, Salipiger mucosus, Salipiger species, Sinorhizobium americanum, Sinorhizobium fredii, Sinorhizobium meliloti, Sinorhizobium species, Skermanella species, Skermanella stibiiresistens, Stappia aggregate, Stappia species, Thauera humireducens, Thauera species, Variovorax paradoxus, Variovorax species, Xanthobacter autotrophicus, Xanthobacter species, and combinations thereof.

In some aspects, the bacteria is Xanthobacter autotrophicus.

In some aspects, the nitrogen fixing microorganism is a fungi.

In some aspects, the fungi is selected from the group consisting of Glomus aggregatum, Glomus intraradices, Glomus mosseae, Glomus etunicatum, Trichoderma reesei, Candida utilis, Penicillium bilaiae, Saccharomyces cerevisiae, Trichoderma harzianum, Trichoderma virens, and combinations thereof.

In some aspects, the composition comprises at least about 80% v/v, at least about 85% v/v, at least about 90% v/v, at least about 95% v/v, or at least about 99% v/v of the diluent.

In some aspects, the composition comprises at least about 1% v/v, at least about 5% v/v, at least about 10% v/v, at least about 15% v/v, or at least about 20% v/v of the microorganism.

In some aspects, the liquid formulation is concentrated to remove water.

Certain aspects of the disclosure provide a kit comprising any of the compositions disclosed herein.

In some aspects, the microorganism is in a dry formulation.

In some aspects, the diluent is in a dry formulation.

Certain aspects of the disclosure provide a method of preparing a biofertilizer comprising: i) obtaining an extract or slurry from a biomass to form a diluent, wherein the biomass is derived from plant, yeast, insect, crustacean or algae; and ii) combining the diluent with a nitrogen fixing microorganism to form a biofertilizer.

In some aspects, the extract or slurry from a biomass is obtained by steeping the biomass, by homogenizing the biomass, or juicing the biomass.

In some aspects, the method further comprises dehydrating the diluent after step i) to form a dry formulated diluent and rehydrating the dry formulated diluent before combining the diluent with the nitrogen fixing microorganism in step ii).

Certain aspects of the disclosure provide a method of preparing a biofertilizer comprising: i) adding a plant biomass or an algae biomass to a hot water bath to obtain a tea mixture; ii) filtering the tea mixture and the plant biomass or an algae biomass to obtain a filtrate; iii) centrifuging the filtrate to obtain a tea extract; iv) filtering the tea extract to obtain a tea supernatant; and v) combining the tea supernatant with a microorganism to form a biofertilizer.

Certain aspects of the disclosure provide a method of preparing a biofertilizer comprising: i) providing a diluent in a dry formulation and a microorganism in a dry formulation, wherein the diluent is derived from a plant or algae, wherein the diluent has not been subjected to fermentation; ii) rehydrating the diluent in a dry formulation to form a rehydrated diluent; iii) rehydrating the microorganism in a dry formulation to form a rehydrated nitrogen fixing microorganism; and iv) combining the rehydrated diluent and the rehydrated nitrogen fixing microorganism to form a biofertilizer.

Certain aspects of the disclosure provide a method of increasing nitrogen utilization efficiency of a plant, comprising administering to the plant any of the compositions disclosed herein.

In some aspects, nitrogen utilization efficiency is measured as: (Yield Nt)/(fertilizer N), wherein Yield Nt is the amount of nitrogen incorporated a crop, and fertilizer N is the amount of fertilizer nitrogen added to the crop.

Certain aspects of the disclosure provide a method of increasing biomass, fruit quality, growth rate, lateral root density, or yield of a plant comprising administering to the plant any of the compositions disclosed herein.

In some aspects, the biomass is measured via aboveground fresh weight.

In some aspects, the biomass is measured via aboveground dry weight.

Certain aspects of the disclosure provide a method of increasing resistance to growth disease in a plant comprising administering to the plant any of the compositions disclosed herein.

In some aspects, the growth disease is a fungal disease.

In some aspects, the fungal disease is powdery mildew, downy mildew, or blight.

In some aspects, the growth disease is tip burn.

Certain aspects of the disclosure provide a method of increasing resistance to chemical over application in a plant comprising administering to the plant any of the compositions disclosed herein.

In some aspects, the composition comprises between about 1×10⁹ CFU/mL and about 4×10⁹ CFU/mL of the microbial cells.

In some aspects, the composition comprises at least about 1×10⁹ CFU/mL of the microbial cells.

In some aspects, the composition comprises about 5×10⁸ CFU/mL of a first microbial species, and about 5×10⁸ CFU/mL of a second microbial species.

In some aspects, the first microbial species is Xanthobacter autotrophicus.

In some aspects, the second microbial species is Pseudomonas fluorescens, Rhodopseudomonas palustris, Azospirillum lipoferum, or Cupriavidus necator.

Certain aspects of the disclosure provide a method of improving the efficacy of a microorganism in a biofertilizer comprising contacting the microorganism with a diluent, wherein the diluent is derived from a plant, yeast, insect, or algae, wherein the microorganism is a nitrogen-fixing microorganism.

In some aspects, the diluent is derived from a plant.

In some aspects, the plant is selected from a group consisting of a coffee plant, a carrot, a potato, an orange, a banana, an alfalfa grass, a tomato, a grape, a rice, or a maple tree.

In some aspects, the diluent is derived from a peel of a banana, a peel of an orange, a coffee ground, a husk of a rice plant, or a syrup from a maple tree.

In some aspects, the diluent is derived from an algae.

In some aspects, the diluent is derived from a yeast.

In some aspects, the yeast is a brewing yeast.

In some aspects, the yeast is a spent brewing yeast.

In some aspects, the diluent is derived from an insect.

In some aspects, the insect is a bee.

In some aspects, the diluent is derived from honey or bee's wax.

### BRIEF DESCRIPTION OF THE DRAWINGS/FIGURES

FIG. 1A (FIG. 1A) provides nitrogen utilization efficiency (NUE) for treatment groups containing 1) 80% Growth Standard Practice (GSP) recommended Total Nitrogen rate of 100 lbs Nitrogen/acre; 2) 80% GSP+1E9 CFUs Biofertilizer (as used throughout the figures and examples unless otherwise indicated, means a biofertilizer comprising Xanthobacter autotrophicus, e.g. 1×10⁸ CFU/mL of Xanthobacter autotrophicus); 3) 80% GSP+4E9 CFUs Biofertilizer; 4) 80% GSP+1E10 CFUs Biofertilizer; 5) 80% GSP+4E10 CFUs Biofertilizer; 6) 80% GSP+1E8 CFUs Biofertilizer+Diluent #1; 7) 80% GSP+4E8 CFUs Biofertilizer+Diluent #1; 8) 80% GSP+E9 CFUs Biofertilizer+Diluent #1; 9) 80% GSP+4E9 CFUs Biofertilizer+Diluent #1; and 10) 100% GSP. An increase in NUE suggests either an increased efficiency of nitrogen uptake or a greater input of nitrogen into the system from nitrogen fixation.

FIG. 1B (FIG. 1B) provides above-ground dry weight (grams) for treatment groups containing 1) 80% Growth Standard Practice (GSP) recommended Total Nitrogen rate of 100 lbs Nitrogen/acre; 2) 80% GSP+1E9 CFUs Biofertilizer; 3) 80% GSP+4E9 CFUs Biofertilizer; 4) 80% GSP+1E10 CFUs Biofertilizer; 5) 80% GSP+4E10 CFUs Biofertilizer; 6) 80% GSP+1E8 CFUs Biofertilizer+Diluent #1; 7) 80% GSP+4E8 CFUs Biofertilizer+Diluent #1; 8) 80% GSP+1E9 CFUs Biofertilizer+Diluent #1; 9) 80% GSP+4E9 CFUs Biofertilizer+Diluent #1; and 10) 100% GSP.

FIG. 1C (FIG. 1C) provides above-ground fresh weight (grams) for treatment groups containing 1) 80% Growth Standard Practice (GSP) recommended Total Nitrogen rate of 100 lbs Nitrogen/acre; 2) 80% GSP+1E9 CFUs Biofertilizer; 3) 80% GSP+4E9 CFUs Biofertilizer; 4) 80% GSP+1E10 CFUs Biofertilizer; 5) 80% GSP+4E10 CFUs Biofertilizer; 6) 80% GSP+1E8 CFUs Biofertilizer+Diluent #1; 7) 80% GSP+4E8 CFUs Biofertilizer+Diluent #1; 8) 80% GSP+1E9 CFUs Biofertilizer+Diluent #1; 9) 80% GSP+4E9 CFUs Biofertilizer+Diluent #1; and 10) 100% GSP.

FIG. 1D (FIG. 1D) provides below-ground dry weight (grams) for treatment groups containing 1) 80% Growth Standard Practice (GSP) recommended Total Nitrogen rate of 100 lbs Nitrogen/acre; 2) 80% GSP+1E9 CFUs Biofertilizer; 3) 80% GSP+4E9 CFUs Biofertilizer; 4) 80% GSP+1E10 CFUs Biofertilizer; 5) 80% GSP+4E10 CFUs Biofertilizer; 6) 80% GSP+1E8 CFUs Biofertilizer+Diluent #1; 7) 80% GSP+4E8 CFUs Biofertilizer+Diluent #1; 8) 80% GSP+1E9 CFUs Biofertilizer+Diluent #1; 9) 80% GSP+4E9 CFUs Biofertilizer+Diluent #1; and 10) 100% GSP.

FIG. 2 (FIG. 2) provides the fresh weight (grams) for lettuce treated with 1) low nitrogen baseline (Low N UTC) representing 80% GSP; 2) 80% GSP+1:10 ratio of 1E9 Biofertilizer in Diluent #1 (AK1); 3) 80% GSP+1:10 ratio of 1E10 Biofertilizer in Diluent #1 (AK2); 4) 80% GSP+1E9 Biofertilizer (K1); 5) 80% GSP+1E10 Biofertilizer (K2); and 6) high nitrogen baseline (High N UTC) representing 100% GSP.

FIG. 3A (FIG. 3A) provides NUE % for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs biofertilizer+Diluent #1 (10× dilution in water); and 6) 1E7 CFUs Biofertilizer+Diluent #1 (100× dilution in water).

FIG. 3B (FIG. 3B) provides plant height average (mm) for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs Biofertilizer+Diluent #1 (10× dilution in water); and 6) 1E7 CFUs Biofertilizer+Diluent #1 (100× dilution in water).

FIG. 3C (FIG. 3C) provides leaf area (mm²) for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs Biofertilizer+Diluent #1 (10× dilution in water); and 6) 1E7 CFUs Biofertilizer+Diluent #1 (100× dilution in water).

FIG. 3D (FIG. 3D) provides above-ground fresh biomass (g) for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs Biofertilizer+Diluent #1 (10× dilution in water); and 6) 1E7 CFUs Biofertilizer+Diluent #1 (100× dilution in water).

FIG. 3E (FIG. 3E) provides above-ground dry biomass (g) for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs Biofertilizer+Diluent #1 (10× dilution in water); and 6) 1E7 CFUs Biofertilizer+Diluent #1 (100× dilution in water).

FIG. 3F (FIG. 3F) provides below-ground dry biomass (g) for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs Biofertilizer+Diluent #1 (10× dilution in water); and 6) 1E7 CFUs Biofertilizer+Diluent #1 (100× dilution in water).

FIG. 3G (FIG. 3G) provides root:shoot ratio for treatment groups containing 1) untreated control at 80% GSP and 100% GSP; 2) 1E9 CFUs Biofertilizer+Diluent #1; 3) 1E8 CFUs Biofertilizer+Diluent #1 (10× dilution in Diluent #1); 4) 1E7 CFUs Biofertilizer+Diluent #1 (100× dilution in Diluent #1); 5) 1E8 CFUs Biofertilizer+Diluent #1 (10× dilution in water); and 6) 1E7 CFUs Biofertilizer+Diluent #1 (100× dilution in water).

FIG. 4 (FIG. 4) shows lateral root density of lettuce after treatment with standard Biofertilizer with Diluent #1 (D1) or Biofertilizer prepared with tomato extract or orange peel extract at two different concentrations (DE=ions equivalent to D1, ME=ions equivalent to growth media). Statistically significant differences are indicated by uppercase letters based on analysis of variance (ANOVA).

FIGS. 5A-5B (FIGS. 5A-5B) show aboveground fresh biomass (FIG. 5A) and dry biomass (FIG. 5B) observed in lettuce treated with standard Biofertilizer+D1 compared to Biofertilizer prepared with tomato or orange peel extract as diluent. Values are shown as average±standard error (n=12). Statistically significant differences are indicated by uppercase letters based on analysis of variance (ANOVA).

FIGS. 6A-6C (FIGS. 6A-6C) show leaf tissue nitrogen content (FIG. 6A), total leaf nitrogen (FIG. 6B), and nitrogen use efficiency crop (FIG. 6C) data of lettuce grown in pots. Leaf tissue content is reported in percent (w/w). Total leaf nitrogen is reported in mg. Nitrogen use efficiency crop reported as a ratio. Values are shown as average±standard error (n=12). Statistically significant differences are indicated by uppercase letters based on analysis of variance (ANOVA).

FIG. 7 (FIG. 7) shows lateral root density of lettuce after treatment with standard Biofertilizer+D1 or Biofertilizer prepared with various alternative diluent sources tested in Example 8. Statistically significant differences are indicated by uppercase letters based on analysis of variance (ANOVA).

FIG. 8 (FIG. 8) shows lateral root density of lettuce after treatment with standard Biofertilizer+D1 or Biofertilizer prepared with various alternative diluent sources tested in Example 9. Statistically significant differences are indicated by uppercase letters based on analysis of variance (ANOVA).

FIG. 9 (FIG. 9) shows aboveground fresh biomass observed in lettuce treated with standard Biofertilizer+D1 compared to Biofertilizer with cells only, or Biofertilizer prepared using other diluents. Values are shown as average±standard error (n=12). Statistically significant differences are indicated by uppercase letters based on analysis of variance (ANOVA).

FIGS. 10A-10C (FIGS. 10A-10C) show leaf tissue nitrogen content (FIG. 10A), total leaf nitrogen (FIG. 10B), and nitrogen use efficiency crop (FIG. 10C) data of lettuce grown in pots. Leaf tissue content is reported in percent (w/w). Total leaf nitrogen is reported in mg. Nitrogen use efficiency crop reported as a ratio. Values are shown as average±standard error (n=12). Statistically significant differences are indicated by uppercase letters based on analysis of variance (ANOVA).

FIG. 11 (FIG. 11) shows daily CFU/mL of treatments averaged across biological reps and technical plating duplicates (control, no yeast extract with 4 g/L Rice Husk diluent added (“replacement”), or yeast extract+4 g/L Rice Husk diluent added (“Boost”)).

FIG. 12 (FIG. 12) shows OD₆₀₀ PHB values from each treatment corrected for dilution (control, no yeast extract with 4 g/L Rice Husk diluent added (“replacement”), or yeast extract+4 g/L Rice Husk diluent added (“Boost”)).

FIG. 13 (FIG. 13) shows daily CFU/mL of each treatment from each treatment averaged across biological reps (control, no yeast extract with 4 g/L Rice Husk diluent added (“replacement”), or yeast extract+4 g/L Rice Husk diluent added (“Boost”)).

FIG. 14 (FIG. 14) shows a graphical representation of the rice husk diluent formulations.

FIG. 15 (FIG. 15) shows CFU/mL counts at each sampled point of diluent formulations stored at 4° C.

FIG. 16 (FIG. 16) shows CFU/mL counts at each sampled point of diluent formulations stored at room temperature.

FIG. 17 (FIG. 17) shows CFU/mL counts at each sampled point of diluent formulations stored at 25° C.

FIG. 18 (FIG. 18) shows CFU/mL counts at each time point of higher density experiments stored at 25° C.

FIG. 19 (FIG. 19) shows Log 10-Fold change from the initial cell density for each sampled point of higher density experiments. The dotted line indicates a log-fold decline in cell density.

FIG. 20 (FIG. 20) shows lettuce biomass in grams of each diluent treatment from the lettuce tissue culture trials. Statistically significant differences are indicated by uppercase letters based on analysis of variance (ANOVA).

FIG. 21 (FIG. 21) shows percent biomass relative to the Biofertilizer+D1 control of each diluent treatment from the lettuce tissue culture trials.

FIG. 22 (FIG. 22) shows lettuce biomass in grams of each diluent treatment from the redefined lettuce tissue culture trials. Statistically significant differences are indicated by uppercase letters based on analysis of variance (ANOVA).

FIG. 23 (FIG. 23) shows percent biomass relative to the Biofertilizer+D1 control of each diluent treatment from the redefined lettuce tissue culture trials.

FIG. 24 (FIG. 24) shows lettuce biomass in grams of each diluent treatment from the lower density lettuce tissue culture trials. Statistically significant differences are indicated by uppercase letters based on analysis of variance (ANOVA).

FIG. 25 (FIG. 25) shows percent biomass relative to the Biofertilizer+D1 control of each diluent treatment from the lower density lettuce tissue culture trials.

FIG. 26 (FIG. 26) shows lateral root density of lettuce after treatment with standard Biofertilizer+D1 or Mixed microbial cultures in diluent. Statistically significant differences are indicated by uppercase letters based on analysis of variance (ANOVA).

FIGS. 27A-27B (FIGS. 27A-27B) show percent yield increase and nitrogen use efficiency for treatments with a combination of GSP, Biofertilizer+D1, and D1 diluent.

FIGS. 28A-28B (FIGS. 28A-28B) show percent yield increase and nitrogen use efficiency for treatments and boosts with a combination of GSP, Biofertilizer+D1, and D1 diluent.

FIGS. 29A-29B (FIGS. 29A-29B) show percent yield increase and nitrogen use efficiency for treatments with a combination of GSP, Biofertilizer+D1, and D1 diluent, with the Application rate equivalent for biofertilizer treatments being 1) 2 ounces equaling 1 pound of Nitrogen, or 2) 8 ounces equaling 1 pound of Nitrogen.

FIGS. 30A-30B (FIGS. 30A-30B) show percent yield increase and nitrogen use efficiency for treatments with 80% GSP, 100% GSP, or 80% GSP+Biofertilizer+Diluent formulation D1, with Biofertilizer CFUs ranging from 1E7 CFUs to 1E9 CFUs.

FIG. 31 (FIG. 31) shows Lateral Root Density measured in number of lateral roots per cm of taproot length. Values show mean±standard error (n=15).

FIG. 32 (FIG. 32) shows aboveground fresh biomass of lettuce grown in tissue culture. Values are reported as average mass in g±standard error (n=8).

FIG. 33 (FIG. 33) shows aboveground fresh biomass of lettuce grown in pots. Values are reported as average mass in g±standard error (n=12).

FIGS. 34A-34C (FIGS. 34A-34C) show leaf tissue nitrogen content (FIG. 34A), total leaf nitrogen (FIG. 34B), and nitrogen use efficiency crop (FIG. 34C) data of lettuce grown in pots. Leaf tissue content is reported in percent (w/w). Total leaf nitrogen is reported in mg. Nitrogen use efficiency crop reported as a ratio. Values are shown as average±standard error (n=12).

FIG. 35 (FIG. 35) shows aboveground vegetative biomass of tomatoes grown in pots. Values are reported as average mass in g±standard error (n=7).

FIGS. 36A-36D (FIGS. 36A-36D) show fruit yield data of tomatoes grown in pots. Total fruit yield per plant reported in average g fruit per plant±standard error (n=7) (FIG. 36A). Red fruit yield per plant reported in average g red fruit per plant±standard error (n=7) (FIG. 36B). Total fruit counts reported in average number of fruits per plant±standard error (n=7) (FIG. 36C). Average fruit size per plant reported in g per fruit±standard error (n=7) (FIG. 36D).

### DETAILED DESCRIPTION

Certain aspects of the disclosure provide a composition comprising i) a diluent and ii) a microorganism. In some aspects, the diluent is derived from a plant, yeast, insect, crustacean or algae. In some aspects, the microorganism is a nitrogen fixing microorganism.

Certain aspects of the disclosure provide a kit comprising any of the compositions disclosed herein.

Certain aspects of the disclosure provide a method of preparing a biofertilizer comprising: i) obtaining an extract or slurry from a biomass to form a diluent, wherein the biomass is derived from plant, yeast, insect, crustacean or algae; and ii) combining the diluent with a nitrogen fixing microorganism to form a biofertilizer.

Certain aspects of the disclosure provide a method of preparing a biofertilizer comprising: i) adding a plant biomass or an algae biomass to a hot water bath to obtain a tea mixture; ii) filtering the tea mixture and the plant biomass or an algae biomass to obtain a filtrate; iii) centrifuging the filtrate to obtain a tea extract; iv) filtering the tea extract to obtain a tea supernatant; and v) combining the tea supernatant with a microorganism to form a biofertilizer.

Certain aspects of the disclosure provide a method of preparing a biofertilizer comprising: i) providing a diluent in a dry formulation and a microorganism in a dry formulation, wherein the diluent is derived from a plant or algae, wherein the diluent has not been subjected to fermentation; ii) rehydrating the diluent in a dry formulation to form a rehydrated diluent; iii) rehydrating the microorganism in a dry formulation to form a rehydrated nitrogen fixing microorganism; and iv) combining the rehydrated diluent and the rehydrated nitrogen fixing microorganism to form a biofertilizer.

Certain aspects of the disclosure provide a method of increasing nitrogen utilization efficiency of a plant, comprising administering to the plant any of the compositions disclosed herein.

Certain aspects of the disclosure provide a method of increasing biomass, fruit quality, growth rate, lateral root density, or yield of a plant comprising administering to the plant any of the compositions disclosed herein.

Certain aspects of the disclosure provide a method of increasing resistance to growth disease in a plant comprising administering to the plant any of the compositions disclosed herein.

Certain aspects of the disclosure provide a method of increasing resistance to chemical over application in a plant comprising administering to the plant any of the compositions disclosed herein.

Certain aspects of the disclosure provide a method of improving the efficacy of a microorganism in a biofertilizer comprising contacting the microorganism with a diluent, wherein the diluent is derived from a plant, yeast, insect, or algae, wherein the microorganism is a nitrogen-fixing microorganism.

### Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference. Incorporation by reference of any such documents shall not be considered an admission that the incorporated materials are prior art to the present disclosure, or considered as material to the patentability of the present disclosure.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean about, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this application, various embodiments of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range. Furthermore, a recited range should be considered to include the end points of the range. For example, “between about 15 and about 50” should be interpreted to include about 15, about 50, and subranges between as described above.

The terms “comprises,” “comprising,” “includes,” “including,” “having,” and their conjugates as used herein are interchangeable and mean “including but not limited to.” It is understood that wherever aspects are described herein with the language “comprising,” otherwise analogous aspects described in terms of “consisting of” and/or “consisting essentially of” are also provided.

The term “consisting of” as used herein means including and limited to.

The term “consisting essentially of” as used herein means the specified material of a composition, or the specified steps of a method, and those additional materials or steps that do not materially affect the basic characteristics of the material or method.

As used herein, the term “effective amount” in terms of a biofertilizer will depend upon a variety of factors, including, for example, percent viability of cells in the biofertilizer, concentration of cells in the biofertilizer, the levels of nutrients, including ammonia and carbon sources (e.g., PHB), and whether the biofertilizer is in the form of a liquid cell suspension or comprises a solid biomass component, such as soils, plant materials, or inert materials. A person of ordinary skill in the art will be able to determine an effective amount taking into account these variables. For purposes of the instant disclosure, an effective amount of a biofertilizer means an amount of the biofertilizer that is sufficient to result in an enhanced property or characteristic of a soil microbiome and/or a crop or plant that is statistically greater than the same property or characteristic in the absence of the biofertilizer, such as, for example, increased crop yield, increased fruit or vegetable yield or root storage mass, increased carbon and/or nitrogen availability in the microbiome. In some aspects, the property or characteristic (e.g., crop or plant yield or yield quality) enhanced by the biofertilizer is observed with at least a 5%, or at least a 6%, or 7%, or 8%, or 9%, or 10%, or 25%, or 50%, or 75%, or 100%, or 200%, or 300%, or 400%, or 500%, or 1000%, or 1250%, or 1500%, or 2000%, or more increase over the same property or characteristic established in the absence of the biofertilizer.

As used herein, the term “microbiome” refers to the collection of all microorganisms living in a particular environment, including in the soil surrounding and/or interacting with the root of a plant.

As used herein, the term “biofertilizer” refers to a preparation containing living cells or latent cells of microorganisms that help plants (e.g., crop plants) grow. The term also refers to a preparation containing living cells or latent cells of microorganisms that help to feed and/or enhance the soil microbiome. The term also refers to a preparation containing living cells or latent cells of microorganisms that produce chemicals (including but not limited to nitrate, ammonia, phosphorus) to directly or indirectly provide nutrition to plants (e.g., crop plants), or to directly or indirectly signal plant or microbial pathways to the benefit of the plant (e.g. crop plants), in the soil, soilless substrate, or other growth medium. In some aspects, the biofertilizer can be a preparation containing living cells or latent cells of microorganisms, or having enhanced accumulation of microbial intracellular storage compound (MISC), or a combination thereof. In some aspects, the MISC accumulated by the microorganism comprises a polyhydroxyalkanoate (PHA), a polyphosphate (PolyP), a lipid, or a combination thereof. In some aspects, the MISC is a PHA. In some aspects, the PHA is polyhydroxybutyrate (PHB), poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

As used herein, the term “nitrogenase” refers to an enzyme that is produced by certain specialized bacteria called nitrogen-fixing bacteria, such as cyanobacteria and Xanthobacter (e.g., X. autotrophicus), which are responsible for reducing atmospheric nitrogen (N₂) to ammonia (NH₃) as part of the nitrogen cycle.

As used herein, the term “stable” or “stability” refers to the viability of the microorganisms in biofertilizers. Biofertilizers comprise living microorganisms, unlike chemical fertilizers. Biofertilizers themselves can comprise the source of nutrients, and/or can help the crops or plants in accessing the nutrient available in its surrounding environment. The viability of the microorganisms during production, formulation, storage, transportation, distribution and field application is directly related to the performance and potentials of a biofertilizer. A range of commercial biofertilizer formulation strategies can be applied to ensure maximum viability of the microorganisms used in such formulations. These strategies include: (i) optimization of biofertilizer formulation, (ii) application of thermo-tolerant/drought-tolerant/genetically modified strains and, (iii) application of liquid biofertilizer. For convenience of application, a carrier material can be used as a vehicle for the microorganisms to be used as biofertilizer. Moreover, such materials can have a role in maintaining the viability (shelf-life) of the microorganisms prior to its release into the field as well as they also provide a suitable microenvironment for rapid growth of the organisms upon their release. A carrier can be a material, such as peat, vermiculite, lignite powder, clay, talc, rice bran, seed, rock phosphate pellet, charcoal, soil, paddy straw compost, wheat bran or a mixture of such materials. In common practice, for a better shelf-life of a biofertilizer formulation, a carrier or a mixture of carrier materials can be selected based on the viability of the microorganisms mixed with them. Similarly, pre-sterilization of the carrier material and its enrichment with nutrients can be another strategy for improving the shelf-life by allowing the microorganism to maintain and grow in a non-competitive microenvironment. Sucrose, maltose, trehalose, molasses, glucose, and/or glycerol can be supplementary nutrients or cell protectants commonly used with a carrier material to ensure maximum cell viability and extended shelf-life. Liquid biofertilizer formulations can be considered as one potential strategy for improving the shelf-life of biofertilizer. Unlike solid carrier based biofertilizers, liquid formulations allow the manufacturer to include sufficient amount of nutrients, cell protectant, and inducers responsible for cell/spore/cyst formation to ensure prolonged shelf-life. The shelf-life of common solid carrier based biofertilizers can be around six months; however, it could be as high as two years for a liquid formulation. Further, solid carrier based biofertilizers can be less thermo-tolerant, whereas liquid formulations can tolerate the temperature as high as 55° C. Hence, improved shelf-life can be achieved formulating biofertilizers into liquid formulations.

The term “derived from” as used herein refers to a component that is isolated from or made using a plant (e.g., an extract or tea), yeast (e.g. spent brewing yeast), insect (e.g., honey), or algae. For example, a diluent that is derived from a plant can include a tea obtained from plant biomass.

The term “growth disease” as used herein refers to any disease or condition that adversely affects the normal growth of a plant. For example, growth diseases may include a fungal disease (e.g., powdery mildew, downy mildew, or blight) or tip burn.

The term “Chemical Over Application” as used herein refers to over application of fertilizer, pesticides, or other chemicals used during agriculture practices which may lead to negative consequences. For example, over application of fertilizers may lead to decreased soil health and plant fertility (e.g., by depletion of essential soil nutrients and minerals or increased salt content).

The term “efficacy of a microorganism” as used herein refers to the ability of microorganism to produce a desired biological effect in a plant (e.g., increased nitrogen utilization efficiency, increased biomass, increased fruit quality, increased growth rate, increased lateral root density, increased yield, increased resistance to growth disease, or increased resistance to chemical overapplication).

The term “v/v” or “% v/v” as used herein refers to the concentration of one liquid in another liquid obtained by comparing the relative volumes of one liquid and the other liquid. For example, a composition comprising 80% v/v diluent contains 80 mL of diluent per 100 mL of total solution (e.g., a composition disclosed herein).

The term “w/w” or “% w/w” as used herein refers to proportions by weight, and means the ratio of the weight of one substance (e.g., a dry formulated diluent) in a composition to the total weight of the composition, or the weight of one substance in the composition to the weight of another substance of the composition. For example, reference to a composition that comprises 80% w/w diluent means that 80% of the composition's weight is composed of diluent (e.g., such a composition having a weight of 100 mg would contain 80 mg of diluent) and the remainder of the weight of the composition (e.g., 20 mg in the example) is composed of other ingredients (e.g., a microorganism).

As used herein, “spray drying” refers to processes that produce dry powders from a liquid or slurry. “Spray dried,” when referring to a substance, refers to a substance that has undergone the spray drying process.

As used herein, “steeping” refers to soaking in a liquid to soften, cleanse or extract some constituent.

As used herein, “homogenizing” refers to intensive mixing to obtain a soluble suspension or emulsion. Homogenization may be performed to break down the biomass (e.g., a plant, yeast, insect, crustacean or algae) into smaller sizes. This can be achieved by forcing the biomass (e.g., a plant, yeast, insect, crustacean or algae) through small holes at high pressure.

As used herein, “diluent” refers to a substance used for dilution (e.g., for diluting a microorganism). In some aspects, the diluent is derived from a plant (e.g., the seeds, the leaves, the roots, the shoots, the flower, or the fruit of the plant, or the entire plant), yeast, insect, crustacean or algae.

Compositions and/or Formulations

Certain aspects of the disclosure provide a composition comprising i) a diluent and ii) a microorganism. In some aspects, the diluent is derived from a plant, yeast, insect, crustacean or algae. In some aspects, the microorganism is a nitrogen fixing microorganism.

In some aspects, the diluent is in a dry formulation.

In some aspects, the diluent is in a powder.

In some aspects, the diluent is lyophilized.

In some aspects, the diluent is freeze-dried. Freeze-drying involves, for example, the removal of liquid from the diluent.

In some aspects, freeze-drying the diluent comprises placing the frozen diluent into a freeze-drier.

In some aspects, the microorganism is in a dry formulation.

In some aspects, the microorganism is in a powder.

In some aspects, the microorganism is lyophilized.

In some aspects, the microorganism is freeze-dried. Freeze-drying involves, for example, the removal of liquid from the microorganism.

In some aspects, freeze-drying the microorganism comprises placing the frozen diluent into a freeze-drier.

In some aspects, the diluent has been dried by spray drying. In some aspects, the microorganism has been dried by spray drying. Spray drying generally produces small liquid droplets of specific sizes from a liquid or slurry using a spray nozzle or atomizer. After the droplets exit the nozzle or atomizer, they are dried, generally using hot air, to form a powder. Machines known as spray dryers are normally used for this process.

Diluent or microorganisms (generally liquid, but also semi-solid or solid) may be dried, dehydrated or desiccated using a variety of methods. In some examples, a liquid sample may be left open so that moisture from the sample is evaporated into the air. This may be called air drying. In some examples, a gas stream (e.g., air) may apply heat to the sample by convection and moisture/vapor is removed as humidity. In some aspects, vacuum drying, where heat is supplied to the sample by conduction, radiation, or microwaves, vapor is produced and carried away by a vacuum system, may be used. In some aspects, drum drying, where a surface supplies heat to the sample, vapor is produced and carried away by an aspirator, may be used. In some aspects, a dried sample may be produced by draining (e.g., centrifugation to mechanically extract a solvent).

In some aspects, both the diluent and microorganisms are in a dry formulation (e.g., both before being combined to form a composition, and within the composition).

In some aspects, both the diluent and microorganisms are in a liquid formulation (e.g., both before being combined to form a composition, and within the composition).

In some aspects, the diluent is in a liquid formulation and the microorganisms are in a dry formulation prior to being combined to form a composition.

In some aspects, the diluent is in a dry formulation and the microorganisms are in a liquid formulation prior to being combined to form a composition.

In some aspects, the dry formulation of the diluent or microorganism may have a moisture content of less than about 50%, about 40%, about 30%, about 25%, about 20%, about 15%, about 12%, about 10%, about 9%, about 8%, about 7%, about 6%, about 5%, about 4%, about 3%, about 2%, or about 1%.

In some aspects, the dry formulation of the diluent or microorganism may have a moisture content of about 50%, about 40%, about 30%, about 25%, about 20%, about 15%, about 12%, about 10%, about 9%, about 8%, about 7%, about 6%, about 5%, about 4%, about 3%, about 2%, or about 1%.

In some aspects, the dry formulation of the diluent or microorganism may have a moisture content of less than 50%, 40%, 30%, 25%, 20%, 15%, 12%, 10%, 9%, 8%, 7%, 6%, 5%, 4%, 3%, 2%, or 1%.

In some aspects, the diluent is in a liquid formulation.

In some aspects, the microorganism is in a liquid formulation.

In some aspects, the composition further comprises one, two, three, four, five, six, or more microorganisms.

In some aspects, the diluent is derived from a plant.

In some aspects, the plant is selected from a group consisting of a coffee plant, a carrot, a potato, a citrus plant, a banana, an alfalfa grass, a tomato, a grape, a rice, a maple tree, and combinations thereof.

In some aspects, the diluent is derived from plant waste or a plant byproduct.

In some aspects, the diluent is derived from a peel of a banana, a rind of a citrus plant, a coffee ground, waste from a grape, a husk of a rice plant, or a maple syrup from a maple tree, or combinations thereof.

In some aspects, the diluent comprises an extract selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least two extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least three extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least four or more extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises an alfalfa extract.

In some aspects, the composition comprises between about 15 g/L and about 50 g/L, between about 15 g/L and about 45 g/L, between about 15 g/L and about 40 g/L, between about 15 g/L and about 35 g/L, between about 15 g/L and about 30 g/L, between about 15 g/L and about 25 g/L, between about 15 g/L and about 20 g/L, between about 20 g/L and about 50 g/L, between about 25 g/L and about 50 g/L, between about 30 g/L and about 50 g/L, between about 35 g/L and about 50 g/L, between about 40 g/L and about 50 g/L, between about 45 g/L and about 50 g/mL, between about 20 g/L and about 40 g/L, between about 21 g/L and about 41 g/L, or between about 25 g/L and about 35 g/L of alfalfa extract.

In some aspects, the composition comprises about 15 g/L, about 20 g/L, about 21 g/L, about 25 g/L, about 30 g/L, about 35 g/L, about 40 g/L, about 45 g/L, or about 50 g/L of alfalfa extract.

In some aspects, the diluent comprises a tomato extract.

In some aspects, the composition comprises between about 0.15 g/L and about 1.5 g/L, between about 0.2 g/L and about 1.5 g/L, between about 0.218 g/L and about 1.5 g/L, between about 0.25 g/L and about 1.5 g/L, between about 0.3 g/L and about 1.5 g/L, between about 0.5 g/L and about 1.5 g/L, between about 0.75 g/L and about 1.5 g/L, between about 1.0 g/L and about 1.5 g/L, between about 1.15 g/L and about 1.5 g/L, between about 0.2 g/L and about 1.15 g/L, between about 0.2 g/L and about 1.0 g/L, between about 0.2 g/L and about 0.75 g/L, between about 0.2 g/L and about 0.5 g/L, between about 0.2 g/L and about 0.4 g/L, between about 0.2 g/L and about 0.3 g/L, between about 0.218 g/L and about 0.3 g/L, or between about 0.2 g/L and about 1.15 g/L of tomato extract.

In some aspects, the composition comprises about 0.15 g/L, about 0.2 g/L, about 0.218 g/L, about 0.25 g/L, about 0.3 g/L, about 0.5 g/L, about 0.75 g/L, about 1.0 g/L, about 1.15 g/L, or about 1.5 g/L of tomato extract.

In some aspects, the diluent comprises an orange extract.

In some aspects, the composition comprises between about 2 g/L and about 4 g/L, between about 2.198 g/L and about 4 g/L, between about 2.25 g/L and about 4 g/L, between about 2.5 g/L and about 4 g/L, between about 3 g/L and about 4 g/L, between about 3.5 g/L and about 4 g/L, between about 3.558 g/L and about 4 g/L, between about 2 g/L and about 3.558 g/L, between about 2 g/L and about 3.5 g/L, between about 2 g/L and about 3 g/L, between about 2 g/L and about 2.5 g/L, or between about 2 g/L and about 2.198 g/L of orange extract.

In some aspects, the composition comprises about 2 g/L, about 2.198 g/L, about 2.25 g/L, about 2.5 g/L, about 3 g/L, about 3.5 g/L, or about 4 g/L of orange extract.

In some aspects, the diluent comprises a rice extract.

In some aspects, the composition comprises between about 5 g/L and about 12 g/L, between about 5 g/L and about 11 g/L, between about 5 g/L and about 10.55 g/L, between about 5 g/L and about 10 g/L, between about 5 g/L and about 9 g/L, between about 5 g/L and about 8 g/L, between about 5 g/L and about 7 g/L, between about 5 g/L and about 6 g/L, between about 5 g/L and about 5.26 g/L, between about 5.26 g/L and about 12 g/L, between about 6 g/L and about 12 g/L, between about 7 g/L and about 12 g/L, between about 8 g/L and about 12 g/L, between about 9 g/L and about 12 g/L, between about 10 g/L and about 12 g/L, between about 10.55 g/L and about 12 g/L, or between about 11 g/L and about 12 g/L of rice extract.

In some aspects, the composition comprises about 5 g/L, about 5.26 g/L, about 7 g/L, about 8 g/L, about 9 g/L, about 10 g/L, about 10.55 g/L, about 11 g/L, or about 12 g/L of rice extract.

In some aspects, the diluent is derived from an algae.

In some aspects, the diluent comprises a Chlorella extract.

In some aspects, the composition comprises between about 1 g/L and about 5 g/L, between about 2 g/L and about 5 g/L, between about 3 g/L and about 5 g/L, between about 4 g/L and about 5 g/L, between about 1 g/L and about 4 g/L, between about 1 g/L and about 3 g/L, between about 1 g/L and about 2 g/L, between about 2 g/L and about 4 g/L of Chlorella extract.

In some aspects, the composition comprises about 1 g/L, about 2 g/L, about 3 g/L, about 4 g/L or about 5 g/L of Chlorella extract.

In some aspects, the diluent is derived from a yeast.

In some aspects, the yeast is a brewing yeast.

In some aspects, the yeast is a spent brewing yeast.

In some aspects, the diluent comprises a yeast extract.

In some aspects, the composition comprises between about 1 g/L and about 5 g/L, between about 2 g/L and about 5 g/L, between about 3 g/L and about 5 g/L, between about 4 g/L and about 5 g/L, between about 1 g/L and about 4 g/L, between about 1 g/L and about 3 g/L, between about 1 g/L and about 2 g/L, between about 2 g/L and about 4 g/L of yeast extract.

In some aspects, the composition comprises about 1 g/L, about 2 g/L, about 3 g/L, about 4 g/L or about 5 g/L of yeast extract.

In some aspects, the diluent is derived from an insect.

In some aspects, the insect is a bee.

In some aspects, the diluent is derived from honey or bee's wax.

In some aspects, the diluent is derived from the chitosan of an insect.

In some aspects, the composition comprises between about 25 mg/L and about 600 mg/L, between about 50 mg/L and about 600 mg/L, between about 75 mg/L and about 600 mg/L, between about 100 mg/L and about 600 mg/L, between about 200 mg/L and about 600 mg/L, between about 300 mg/L and about 600 mg/L, between about 400 mg/L and about 600 mg/L, between about 500 mg/L and about 600 mg/L, between about 50 mg/L and about 500 mg/L, between about 25 mg/L and about 500 mg/L, between about 25 mg/L and about 400 mg/L, between about 25 mg/L and about 300 mg/L, between about 25 mg/L and about 200 mg/L, between about 25 mg/L and about 100 mg/L, between about 25 mg/L and about 75 mg/L, or between about 25 mg/L and about 50 mg/L of chitosan.

In some aspects, the composition comprises about 25 mg/L, about 50 mg/L, about 75 mg/L, about 100 mg/L, about 200 mg/L, about 300 mg/L, about 400 mg/L, about 500 mg/L, or about 600 mg/L of chitosan.

In some aspects, the diluent comprises a Chlorella extract and a yeast extract.

In some aspects, the microorganism is a bacteria.

In some aspects, the bacteria is selected from the group consisting of Acidiphilium multivorum, Acidiphilium species, Alcaligenes paradoxus, Alcaligenes species, Arthrobacter species, Azoarcus indigens, Azohydromonas australica, Azohydromonas lata, Azohydromonas species, Azorhizobium caulinodans, Azospirillium brasiliense, Azospirillium spp., Azospirillum amazonsense, Azospirillum lipoferum, Azospirillum lipoferum (RSAL0111), Azospirillum species, Azospirillum thiophilum, Azotobacter chroococum (MCC 0055), Azotobacter spp., Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beggiatoa species, Beijerinckia mobilis, Beijerinckia species, Bradyrhizobium elnakii, Bradyrhizobium japonicum, Bradyrhizobium japonicum (strain USDA 122), Bradyrhizobium species, Burkholderia species, Burkholderia vietnameiensis, Cupriavidus necator, Cupriavidus species, Cyanobacteria species, Derxia gummosa, Derxia species, Gluconacetobacter diazotrophicus, Gluconacetobacter diazotrophicus (MCC 0046), Herbaspirillum autrotrophicum, Herbaspirillum frisingense (MCC 0052), Herbaspirillum species, Hydrogenophaga pseudoflava, Hydrogenophaga species, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Mesorhizobium species, Methylibium petroleiphilum, Methylibium species, Methylocapsa aurea, Methylocapsa species, Methyloferula species, Methyloferula stellate, Methyloversatilis species, Methyloversatilis universalis, Microcyclus aquaticus, Microcyclus ebruneus, Microcyclus species, Nitrosococcus oceani, Nitrosococcus species, Nitrosomonas communis, Nitrospirillum amazonense, Nocardia autotrophica, Nocardia opaca, Nocardia species, Oligotropha carboxidovorans, Oligotropha species, Paenibacillus durus (MCC 0046), Pannonibacter phragmitetus, Pannonibacter species, Paracoccus denitrificans, Paracoccus pantrophus, Paracoccus species, Paracoccus yeei, Pelagibaca bermudensis, Pelagibaca species, Pseudomonas facilis, Pseudomonas fluorescens, Pseudomonas species, Pseudooceanicola atlanticus, Pseudooceanicola species, Ralstonia eutropha, Ralstonia species, Renobacter species, Renobacter vacuolatum, Rhizobium gallicum, Rhizobium japonicum, Rhizobium japonicum (MCC 0071), Rhizobium leguminosarum, Rhizobium leguminosarum biovar viciae, Rhizobium species, Rhizobium spp., Rhodobacter capsulatus, Rhodobacter species, Rhodobacter sphaeroides, Rhodomicrobium species, Rhodomicrobium vannielii, Rhodopseudomonas palustris, Rubrivivax gelatinosus, Rubrivivax species, Salipiger mucosus, Salipiger species, Sinorhizobium americanum, Sinorhizobium fredii, Sinorhizobium meliloti, Sinorhizobium species, Skermanella species, Skermanella stibiiresistens, Stappia aggregate, Stappia species, Thauera humireducens, Thauera species, Variovorax paradoxus, Variovorax species, Xanthobacter autotrophicus, Xanthobacter species, and combinations thereof.

In some aspects, the bacteria is Xanthobacter autotrophicus.

In some aspects, the nitrogen fixing microorganism is a fungi.

In some aspects, the fungi is selected from the group consisting of Glomus aggregatum, Glomus intraradices, Glomus mosseae, Glomus etunicatum, Trichoderma reesei, Candida utilis, Penicillium bilaiae, Saccharomyces cerevisiae, Trichoderma harzianum, Trichoderma virens, and combinations thereof.

In some aspects, the composition comprises at least about 0.1% v/v, at least about 1% v/v, at least about 5% v/v, at least about 10% v/v, at least about 20% v/v, at least about 30% v/v, at least about 40% v/v, at least about 50% v/v, at least about 60% v/v, at least about 70% v/v, at least about 75% v/v, at least about 80% v/v, at least about 85% v/v, at least about 90% v/v, at least about 95% v/v, or at least about 99% v/v of the diluent.

In some aspects, the composition comprises at least 0.1% v/v, at least 1% v/v, at least 5% v/v, at least 10% v/v, at least 20% v/v, at least 30% v/v, at least 40% v/v, at least 50% v/v, at least 60% v/v, at least 70% v/v, at least 75% v/v, at least 80% v/v, at least 85% v/v, at least 90% v/v, at least 95% v/v, or at least 99% v/v of the diluent.

In some aspects, the composition comprises about 0.1% v/v, about 1% v/v, about 5% v/v, about 10% v/v, about 20% v/v, about 30% v/v, about 40% v/v, about 50% v/v, about 60% v/v, about 70% v/v, about 75% v/v, about 80% v/v, about 85% v/v, about 90% v/v, about 95% v/v, or about 99% v/v of the diluent.

In some aspects, the composition comprises between about 0.1% v/v and about 99% v/v, between about 1% v/v and about 99% v/v, between about 10% v/v and about 99% v/v, between about 20% v/v and about 99% v/v, between about 30% v/v and about 99% v/v, between about 40% v/v and about 99% v/v, between about 50% v/v and about 99% v/v, between about 60% v/v and about 99% v/v, between about 70% v/v and about 99% v/v, between about 75% v/v and about 99% v/v, between about 80% v/v and about 99% v/v, between about 85% v/v and about 99% v/v, between about 90% v/v and about 99% v/v, between about 95% v/v and about 99% v/v, between about 80% v/v and about 95% v/v, between about 80% v/v and about 90% v/v, between about 80% v/v and about 85% v/v, between about 85% v/v and about 95% v/v, between about 85% v/v and about 90% v/v, between about 90% v/v and about 95% v/v, between about 0.1% v/v and about 90% v/v, between about 1% v/v and about 90% v/v, between about 0.1% v/v and about 80% v/v, between about 1% v/v and about 80% v/v, between about 0.1% v/v and about 70% v/v, between about 1% v/v and about 70% v/v, between about 0.1% v/v and about 60% v/v, between about 1% v/v and about 60% v/v, between about 0.1% v/v and about 50% v/v, between about 1% v/v and about 50% v/v, between about 0.1% v/v and about 40% v/v, between about 1% v/v and about 40% v/v, between about 0.1% v/v and about 30% v/v, between about 1% v/v and about 30% v/v, between about 0.1% v/v and about 20% v/v, between about 1% v/v and about 20% v/v, between about 25% v/v and about 75% v/v, between about 25% v/v and about 50% v/v, or between about 50% v/v and about 75% v/v of diluent.

In some aspects, the composition comprises between 0.1% v/v and 99% v/v, between 1% v/v and 99% v/v, between 10% v/v and 99% v/v, between 20% v/v and 99% v/v, between 30% v/v and 99% v/v, between 40% v/v and 99% v/v, between 50% v/v and 99% v/v, between 60% v/v and 99% v/v, between 70% v/v and 99% v/v, between 75% v/v and 99% v/v, between 80% v/v and 99% v/v, between 85% v/v and 99% v/v, between 90% v/v and 99% v/v, between 95% v/v and 99% v/v, between 80% v/v and 95% v/v, between 80% v/v and 90% v/v, between 80% v/v and 85% v/v, between 85% v/v and 95% v/v, between 85% v/v and 90% v/v, between 90% v/v and 95% v/v, between 0.1% v/v and 90% v/v, between 1% v/v and 90% v/v, between 0.1% v/v and 80% v/v, between 1% v/v and 80% v/v, between 0.1% v/v and 70% v/v, between 1% v/v and 70% v/v, between 0.1% v/v and 60% v/v, between 1% v/v and 60% v/v, between 0.1% v/v and 50% v/v, between 1% v/v and 50% v/v, between 0.1% v/v and 40% v/v, between 1% v/v and 40% v/v, between 0.1% v/v and 30% v/v, between 1% v/v and 30% v/v, between 0.1% v/v and 20% v/v, between 1% v/v and 20% v/v, between 25% v/v and 75% v/v, between 25% v/v and 50% v/v, or between 50% v/v and 75% v/v of diluent.

In some aspects, the composition comprises at least about 1% v/v, at least about 5% v/v, at least about 10% v/v, at least about 15% v/v, at least about 20% v/v, at least about 25% v/v, at least about 30% v/v, at least about 35% v/v, at least about 40% v/v, at least about 45% v/v, at least about 50% v/v, at least about 55% v/v, at least about 60% v/v, at least about 65% v/v, at least about 70% v/v, at least about 75% v/v, or at least about 80% v/v of the microorganism.

In some aspects, the composition comprises at least 1% v/v, at least 5% v/v, at least 10% v/v, at least 15% v/v, at least 20% v/v, at least 25% v/v, at least 30% v/v, at least 35% v/v, at least 40% v/v, at least 45% v/v, at least 50% v/v, at least 55% v/v, at least 60% v/v, at least 65% v/v, at least 70% v/v, at least 75% v/v, or at least 80% v/v of the microorganism.

In some aspects, the composition comprises about 1% v/v, about 5% v/v, about 10% v/v, about 15% v/v, about 20% v/v, about 25% v/v, about 30% v/v, about 35% v/v, about 40% v/v, about 45% v/v, about 50% v/v, about 55% v/v, about 60% v/v, about 65% v/v, about 70% v/v, about 75% v/v, or about 80% v/v of the microorganism.

In some aspects, the composition comprises between about 1% v/v and about 20% v/v, between about 1% v/v and about 15% v/v, between about 1% v/v and about 10% v/v, between about 1% v/v and about 5% v/v, between about 5% v/v and about 20% v/v, between about 50% v/v and about 20% v/v, between about 15% v/v and about 20% v/v, between about 50% v/v and about 15% v/v, between about 5% v/v and about 80% v/v, between about 10% v/v and about 150% v/v, between about 1% v/v and about 80% v/v, between about 1% v/v and about 70% v/v, between about 1% v/v and about 60% v/v, between about 1% v/v and about 50% v/v, between about 10% v/v and about 40% v/v, between about 10% v/v and about 30% v/v, between about 30% v/v and about 80% v/v, between about 20% v/v and about 80% v/v, between about 30% v/v and about 80% v/v, between about 40% v/v and about 80% v/v, between about 50% v/v and about 80% v/v, between about 60% v/v and about 80% v/v, between about 70% v/v and about 80% v/v, between about 10% v/v and about 40% v/v, or between about 40% v/v and about 80% v/v of the microorganism.

In some aspects, the composition comprises between 1% v/v and 20% v/v, between 1% v/v and 15% v/v, between 1% v/v and 10% v/v, between 1% v/v and 5% v/v, between 5% v/v and 20% v/v, between 10% v/v and 20% v/v, between 15% v/v and 20% v/v, between 5% v/v and 15% v/v, between 5% v/v and 10% v/v, between 10% v/v and 15% v/v, between 1% v/v and 80% v/v, between 1% v/v and 70% v/v, between 1% v/v and 60% v/v, between 1% v/v and 50% v/v, between 1% v/v and 40% v/v, between 1% v/v and 30% v/v, between 10% v/v and 80% v/v, between 20% v/v and 80% v/v, between 30% v/v and 80% v/v, between 40% v/v and 80% v/v, between 50% v/v and 80% v/v, between 60% v/v and 80% v/v, between 70% v/v and 80% v/v, between 10% v/v and 40% v/v, or between 40% v/v and 80% v/v of the microorganism.

In some aspects, the composition comprises at least about 0.1% w/w, at least about 1% w/w, at least about 10% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of the diluent.

In some aspects, the composition comprises at least 0.1% w/w, at least 1% w/w, at least 10% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w of the diluent.

In some aspects, the composition comprises about 0.1% w/w, about 1% w/w, about 10% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, about 95% w/w, or about 99% w/w of the diluent.

In some aspects, the composition comprises between about 0.1% w/w and about 99% w/w, between about 1% w/w and about 99% w/w, between about 5% w/w and about 99% w/w, between about 10% w/w and about 99% w/w, between about 20% w/w and about 99% w/w, between about 30% w/w and about 99% w/w, between about 40% w/w and about 99% w/w, between about 50% w/w and about 99% w/w, between about 60% w/w and about 99% w/w, between about 70% w/w and about 99% w/w, between about 75% w/w and about 99% w/w, between about 80% w/w and about 99% w/w, between about 90% w/w and about 99% w/w, between about 95% w/w and about 99% w/w, between about 0.1% w/w and about 90% w/w, between about 1% w/w and about 90% w/w, between about 0.1% w/w and about 80% w/w, between about 1% w/w and about 80% w/w, between about 0.1% w/w and about 70% w/w, between about 1% w/w and about 70% w/w, between about 0.1% w/w and about 60% w/w, between about 1% w/w and about 60% w/w, between about 0.1% w/w and about 50% w/w, between about 1% w/w and about 50% w/w, between about 0.1% w/w and about 40% w/w, between about 1% w/w and about 40% w/w, between about 0.1% w/w and about 30% w/w, between about 1% w/w and about 30% w/w, between about 0.1% w/w and about 20% w/w, between about 1% w/w and about 20% w/w, between about 0.1% w/w and about 10% w/w, between about 1% w/w and about 10% w/w, between about 35% w/w and about 65% w/w, between about 40% w/w and about 65% w/w, between about 45% w/w and about 65% w/w, between about 50% w/w and about 65% w/w, between about 55% w/w and about 65% w/w, between about 60% w/w and about 65% w/w, between about 35% w/w and about 60% w/w, between about 35% w/w and about 55% w/w, between about 35% w/w and about 50% w/w, between about 35% w/w and about 45% w/w, between about 35% w/w and about 40% w/w, between about 45% w/w and about 55% w/w, between about 40% w/w and about 60% w/w, or between about 40% w/w and about 50% w/w of the diluent.

In some aspects, the composition comprises between 0.1% w/w and 99% w/w, between 1% w/w and 99% w/w, between 5% w/w and 99% w/w, between 10% w/w and 99% w/w, between 20% w/w and 99% w/w, between 30% w/w and 99% w/w, between 40% w/w and 99% w/w, between 50% w/w and 99% w/w, between 60% w/w and 99% w/w, between 70% w/w and 99% w/w, between 75% w/w and 99% w/w, between 80% w/w and 99% w/w, between 90% w/w and 99% w/w, between 95% w/w and 99% w/w, between 0.1% w/w and 90% w/w, between 1% w/w and 90% w/w, between 0.1% w/w and 80% w/w, between 1% w/w and 80% w/w, between 0.1% w/w and 70% w/w, between 1% w/w and 70% w/w, between 0.1% w/w and 60% w/w, between 1% w/w and 60% w/w, between 0.1% w/w and 50% w/w, between 1% w/w and 50% w/w, between 0.1% w/w and 40% w/w, between 1% w/w and 40% w/w, between 0.1% w/w and 30% w/w, between 1% w/w and 30% w/w, between 0.1% w/w and 20% w/w, between 1% w/w and 20% w/w, between 0.1% w/w and 10% w/w, between 1% w/w and 10% w/w, between 35% w/w and 65% w/w, between 40% w/w and 65% w/w, between 45% w/w and 65% w/w, between 50% w/w and 65% w/w, between 55% w/w and 65% w/w, between 60% w/w and 65% w/w, between 35% w/w and 60% w/w, between 35% w/w and 55% w/w, between 35% w/w and 50% w/w, between 35% w/w and 45% w/w, between 35% w/w and 40% w/w, between 45% w/w and 55% w/w, between 40% w/w and 60% w/w, or between 40% w/w and 50% w/w of the diluent.

In some aspects, the composition comprises at least about 0.1% w/w, at least about 1% w/w, at least about 5% w/w, at least about 10% w/w, at least about 15% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of the microorganism.

In some aspects, the composition comprises at least 0.1% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w of the microorganism.

In some aspects, the composition comprises about 0.1% w/w, about 1% w/w, about 5% w/w, about 10% w/w, about 15% w/w, about 20% w/w, about 25% w/w, about 30% w/w, about 35% w/w, about 40% w/w about 45% w/w, about 50% w/w, about 55% w/w, about 60% w/w, about 65% w/w, about 70% w/w, about 75% w/w, about 80% w/w, about 85% w/w, about 90% w/w, about 95% w/w, or about 99% w/w of the microorganism.

In some aspects, the composition comprises between about 0.1% w/w and about 99% w/w, between about 1% w/w and about 99% w/w, between about 5% w/w and about 99% w/w, between about 10% w/w and about 99% w/w, between about 20% w/w and about 99% w/w, between about 30% w/w and about 99% w/w, between about 40% w/w and about 99% w/w, between about 50% w/w and about 99% w/w, between about 60% w/w and about 99% w/w, between about 70% w/w and about 99% w/w, between about 75% w/w and about 99% w/w, between about 80% w/w and about 99% w/w, between about 90% w/w and about 99% w/w, between about 95% w/w and about 99% w/w, between about 0.1% w/w and about 90% w/w, between about 1% w/w and about 90% w/w, between about 0.1% w/w and about 80% w/w, between about 1% w/w and about 80% w/w, between about 0.1% w/w and about 70% w/w, between about 1% w/w and about 70% w/w, between about 0.1% w/w and about 60% w/w, between about 1% w/w and about 60% w/w, between about 0.1% w/w and about 50% w/w, between about 1% w/w and about 50% w/w, between about 0.1% w/w and about 40% w/w, between about 1% w/w and about 40% w/w, between about 0.1% w/w and about 30% w/w, between about 1% w/w and about 30% w/w, between about 0.1% w/w and about 20% w/w, between about 1% w/w and about 20% w/w, between about 0.1% w/w and about 10% w/w, between about 1% w/w and about 10% w/w, between about 35% w/w and about 65% w/w, between about 40% w/w and about 65% w/w, between about 45% w/w and about 65% w/w, between about 50% w/w and about 65% w/w, between about 55% w/w and about 65% w/w, between about 60% w/w and about 65% w/w, between about 35% w/w and about 60% w/w, between about 35% w/w and about 55% w/w, between about 35% w/w and about 50% w/w, between about 35% w/w and about 45% w/w, between about 35% w/w and about 40% w/w, between about 45% w/w and about 55% w/w, between about 40% w/w and about 60% w/w, or between about 40% w/w and about 50% w/w of the microorganism.

In some aspects, the composition comprises between 0.1% w/w and 99% w/w, between 1% w/w and 99% w/w, between 5% w/w and 99% w/w, between 10% w/w and 99% w/w, between 20% w/w and 99% w/w, between 30% w/w and 99% w/w, between 40% w/w and 99% w/w, between 50% w/w and 99% w/w, between 60% w/w and 99% w/w, between 70% w/w and 99% w/w, between 75% w/w and 99% w/w, between 80% w/w and 99% w/w, between 90% w/w and 99% w/w, between 95% w/w and 99% w/w, between 0.1% w/w and 90% w/w, between 1% w/w and 90% w/w, between 0.1% w/w and 80% w/w, between 1% w/w and 80% w/w, between 0.1% w/w and 70% w/w, between 1% w/w and 70% w/w, between 0.1% w/w and 60% w/w, between 1% w/w and 60% w/w, between 0.1% w/w and 50% w/w, between 1% w/w and 50% w/w, between 0.1% w/w and 40% w/w, between 1% w/w and 40% w/w, between 0.1% w/w and 30% w/w, between 1% w/w and 30% w/w, between 0.1% w/w and 20% w/w, between 1% w/w and 20% w/w, between 0.1% w/w and 10% w/w, between 1% w/w and 10% w/w, between 35% w/w and 65% w/w, between 40% w/w and 65% w/w, between 45% w/w and 65% w/w, between 50% w/w and 65% w/w, between 55% w/w and 65% w/w, between 60% w/w and 65% w/w, between 35% w/w and 60% w/w, between 35% w/w and 55% w/w, between 35% w/w and 50% w/w, between 35% w/w and 45% w/w, between 35% w/w and 40% w/w, between 45% w/w and 55% w/w, between 40% w/w and 60% w/w, or between 40% w/w and 50% w/w of the microorganism.

In some aspects, the liquid formulation is concentrated to remove water.

In some aspects, the composition comprises between about 1×10⁵ CFU/mL and about 1×10¹⁰ CFU/mL of the microbial cells.

In some aspects, the composition comprises between about 1×10⁹ CFU/mL and about 4×10⁹ CFU/mL of the microbial cells.

In some aspects, the composition comprises between about 1×10⁵ CFU/mL and about 1×10¹⁰ CFU/mL of the microbial cells, between about 1×10⁵ CFU/mL and about 1×10⁹ CFU/mL of the microbial cells, between about 1×10⁵ CFU/mL and about 1×10⁸ CFU/mL of the microbial cells, between about 1×10⁵ CFU/mL and about 1×10⁷ CFU/mL of the microbial cells, between about 1×10⁵ CFU/mL and about 1×10⁶ CFU/mL of the microbial cells, between about 1×10⁶ CFU/mL and about 1×10¹⁰ CFU/mL of the microbial cells, between about 1×10⁷ CFU/mL and about 1×10¹⁰ CFU/mL of the microbial cells, between about 1×10⁸ CFU/mL and about 1×10¹⁰ CFU/mL of the microbial cells, between about 1×10⁹ CFU/mL and about 1×10¹⁰ CFU/mL of the microbial cells, between about 1×10⁷ CFU/mL and about 1×10⁹ CFU/mL of the microbial cells, between about 1×10⁹ CFU/mL and about 4×10⁹ CFU/mL of the microbial cells, between about 2×10⁹ CFU/mL and about 4×10⁹ CFU/mL of the microbial cells, between about 3×10⁹ CFU/mL and about 4×10⁹ CFU/mL of the microbial cells, between about 1×10⁹ CFU/mL and about 3×10⁹ CFU/mL of the microbial cells, between about 1×10⁹ CFU/mL and about 2×10⁹ CFU/mL of the microbial cells, or between about 2×10⁹ CFU/mL and about 3×10⁹ CFU/mL of the microbial cells.

In some aspects, the composition comprises at least about 1×10⁹ CFU/mL of the microbial cells.

In some aspects, the composition comprises at least about 1×10⁵ CFU/mL, at least about 1×10⁶ CFU/mL, at least about 1×10⁷ CFU/mL, at least about 1×10⁸ CFU/mL, at least about 5×10⁸ CFU/mL, at least about 1×10⁹ CFU/mL, at least about 2×10⁹ CFU/mL, at least about 3×10⁹ CFU/mL, at least about 4×10⁹ CFU/mL, or at least about 1×10¹⁰ CFU/mL of the microbial cells.

In some aspects, the composition comprises at least 1×10⁵ CFU/mL, at least 1×10⁶ CFU/mL, at least 1×10⁷ CFU/mL, at least 1×10⁸ CFU/mL, at least 5×10⁸ CFU/mL, at least 1×10⁹ CFU/mL, at least 2×10⁹ CFU/mL, at least 3×10⁹ CFU/mL, at least 4×10⁹ CFU/mL, or at least 1×10¹⁰ CFU/mL of the microbial cells.

In some aspects, the composition comprises about 1×10⁵ CFU/mL, about 1×10⁶ CFU/mL, about 1×10⁷ CFU/mL, about 1×10⁸ CFU/mL, about 5×10⁸ CFU/mL, about 1×10⁹ CFU/mL, about 2×10⁹ CFU/mL, about 3×10⁹ CFU/mL, about 4×10⁹ CFU/mL, or about 1×10¹⁰ CFU/mL of the microbial cells.

In some aspects, the composition comprises i) about 1×10⁵ CFU/mL, about 1×10⁶ CFU/mL, about 1×10⁷ CFU/mL, about 1×10⁸ CFU/mL, about 5×10⁸ CFU/mL, about 1×10⁹ CFU/mL, about 2×10⁹ CFU/mL, about 3×10⁹ CFU/mL, about 4×10⁹ CFU/mL, or about 1×10¹⁰ CFU/mL of a first microbial cell; and ii) i) about 1×10⁵ CFU/mL, about 1×10⁶ CFU/mL, about 1×10⁷ CFU/mL, about 1×10⁸ CFU/mL, about 5×10⁸ CFU/mL, about 1×10⁹ CFU/mL, about 2×10⁹ CFU/mL, about 3×10⁹ CFU/mL, about 4×10⁹ CFU/mL, or about 1×10¹⁰ CFU/mL of a second microbial cell.

In some aspects, the composition comprises i) about 5×10⁸ CFU/mL of Xanthobacter autotrophicus and ii) about 5×10⁸ CFU/mL of a second microbial cell selected from Pseudomonas fluorescens, Rhodopseudomonas palustris, Azospirillum lipoferum, or Cupriavidus necator.

### Diluents

In some aspects, the diluent is derived from a plant.

In some aspects, the diluent is derived from the seeds, the leaves, the roots, the shoots, the flower, or the fruit of the plant.

In some aspects, the diluent is derived from part of the plant.

In some aspects, the diluent is derived from the entire plant.

In some aspects, the plant is selected from a group consisting of a coffee plant (e.g., Coffea arabica), a carrot (e.g., Daucus carota sativus), a potato (e.g., Solanum tuberosum), a citrus plant (e.g., plants belonging to the citrus genus, such as Citrus limon), a banana (e.g., Musa acuminata), an alfalfa grass (e.g., Medicago sativa), a tomato (e.g., Solanum lycopersicum), a grape (e.g., Vitis labrusca), a rice (e.g., Oryza sativa), a maple tree (e.g., Acer pseudoplatanus), and combinations thereof.

In some aspects, the diluent is derived from a peel of a banana, a rind of a citrus plant, a coffee ground, waste from a grape, a husk of a rice plant, or a maple syrup from a maple tree.

In some aspects, the diluent is derived from an algae.

In some aspects, the diluent is derived from a yeast.

In some aspects, the yeast is a brewing yeast (e.g., Saccharomyces cerevisiae).

In some aspects, the yeast is a spent brewing yeast.

In some aspects, the diluent is derived from an insect.

In some aspects, the insect is a bee (e.g., Apis mellifera).

In some aspects, the diluent is derived from honey or bee's wax.

In some aspects, the diluent comprises an extract selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least two extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least three extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least four or more extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

Certain aspects of the disclosure provide a method of preparing a biofertilizer comprising: i) obtaining an extract or slurry from a biomass to form a diluent, wherein the biomass is derived from plant, yeast, insect, crustacean or algae; and ii) combining the diluent with a nitrogen fixing microorganism to form a biofertilizer.

In some aspects, the method further comprises dehydrating the diluent after step i) to form a dry formulated diluent and rehydrating the dry formulated diluent before combining the diluent with the nitrogen fixing microorganism in step ii).

In some aspects, the extract or slurry from a biomass is obtained by steeping the biomass, by homogenizing the biomass, or by extracting a juice from the biomass.

Certain aspects of the disclosure provide a method of preparing a biofertilizer comprising: i) adding a plant biomass or an algae biomass to a hot water bath to obtain a tea mixture; ii) filtering the tea mixture and the plant biomass or an algae biomass to obtain a filtrate; iii) centrifuging the filtrate to obtain a tea extract; iv) filtering the tea extract to obtain a tea supernatant; and v) combining the tea supernatant with a microorganism to form a biofertilizer.

Certain aspects of the disclosure provide a method of preparing a biofertilizer comprising: i) providing a diluent in a dry formulation and a microorganism in a dry formulation, wherein the diluent is derived from a plant or algae, wherein the diluent has not been subjected to fermentation; ii) rehydrating the diluent in a dry formulation to form a rehydrated diluent; iii) rehydrating the microorganism in a dry formulation to form a rehydrated nitrogen fixing microorganism; and iv) combining the rehydrated diluent and the rehydrated nitrogen fixing microorganism to form a biofertilizer.

### Microorganisms

In some aspects, the composition comprises a microorganism.

In some aspects, the microorganism is a nitrogen fixing microorganism.

In some aspects, the microorganism does not enhance the availability of soil phosphorus.

In some aspects, the microorganism is not Enterobacter cloacae, Citrobacter fruendii, Comamonas testosteroni, or Psuedomonas putida.

In some aspects, the nitrogen fixing microorganism is a bacteria.

In some aspects, the bacteria is selected from the group consisting of Acidiphilium multivorum, Acidiphilium species, Alcaligenes paradoxus, Alcaligenes species, Arthrobacter species, Azoarcus indigens, Azohydromonas australica, Azohydromonas lata, Azohydromonas species, Azorhizobium caulinodans, Azospirillium brasiliense, Azospirillium spp., Azospirillum amazonsense, Azospirillum lipoferum, Azospirillum lipoferum (RSAL0111), Azospirillum species, Azospirillum thiophilum, Azotobacter chroococum (MCC 0055), Azotobacter spp., Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beggiatoa species, Beijerinckia mobilis, Beijerinckia species, Bradyrhizobium elnakii, Bradyrhizobium japonicum, Bradyrhizobium japonicum (strain USDA 122), Bradyrhizobium species, Burkholderia species, Burkholderia vietnameiensis, Cupriavidus necator, Cupriavidus species, Derxia gummosa, Derxia species, Gluconacetobacter diazotrophicus, Gluconacetobacter diazotrophicus (MCC 0046), Herbaspirillum autrotrophicum, Herbaspirillum frisingense (MCC 0052), Herbaspirillum species, Hydrogenophaga pseudoflava, Hydrogenophaga species, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Mesorhizobium species, Methylibium petroleiphilum, Methylibium species, Methylocapsa aurea, Methylocapsa species, Methyloferula species, Methyloferula stellate, Methyloversatilis species, Methyloversatilis universalis, Microcyclus aquaticus, Microcyclus ebruneus, Microcyclus species, Nitrosococcus oceani, Nitrosococcus species, Nitrosomonas communis, Nitrospirillum amazonense, Nocardia autotrophica, Nocardia opaca, Nocardia species, Oligotropha carboxidovorans, Oligotropha species, Paenibacillus durus (MCC 0046), Pannonibacter phragmitetus, Pannonibacter species, Paracoccus denitrificans, Paracoccus pantrophus, Paracoccus species, Paracoccus yeei, Pelagibaca bermudensis, Pelagibaca species, Pseudomonas facilis, Pseudomonas fluorescens, Pseudomonas species, Pseudooceanicola atlanticus, Pseudooceanicola species, Ralstonia eutropha, Ralstonia species, Renobacter species, Renobacter vacuolatum, Rhizobium gallicum, Rhizobium japonicum, Rhizobium japonicum (MCC 0071), Rhizobium leguminosarum, Rhizobium leguminosarum biovar viciae, Rhizobium species, Rhizobium spp., Rhodobacter capsulatus, Rhodobacter species, Rhodobacter sphaeroides, Rhodomicrobium species, Rhodomicrobium vannielii, Rhodopseudomonas palustris, Rubrivivax gelatinosus, Rubrivivax species, Salipiger mucosus, Salipiger species, Sinorhizobium americanum, Sinorhizobium fredii, Sinorhizobium meliloti, Sinorhizobium species, Skermanella species, Skermanella stibiiresistens, Stappia aggregate, Stappia species, Thauera humireducens, Thauera species, Variovorax paradoxus, Variovorax species, Xanthobacter autotrophicus, Xanthobacter species, and combinations thereof.

In some aspects, the bacteria is Xanthobacter autotrophicus.

In some aspects, the nitrogen fixing microorganism is a fungi.

In some aspects, the fungi is selected from the group consisting of Glomus aggregatum, Glomus Intraradices, Glomus Mosseae, Glomus etunicatum, Trichoderma reesei, Candida utilis, Penicillium bilaiae, Saccharomyces cerevisiae, Trichoderma harzianum, Trichoderma virens, and combinations thereof.

In some aspects, the microorganism is a cyanobacteria.

In some aspects, the microorganism is a nitrogen-fixing microorganism. In some aspects, the nitrogen-fixing microorganism expresses nitrogenase. In some aspects, the nitrogen-fixing microorganism accumulates a microbial intracellular storage compound (MISC). In some aspects, the nitrogen-fixing microorganism expresses nitrogenase and accumulates a MISC. In some aspects, the MISC comprises a polyhydroxyalkanoate (PHA), a polyphosphate (PolyP), or a lipid, or a combination thereof. In some aspects, the MISC is a PHA. In some aspects, the PHA is polyhydroxybutyrate (PHB) poly-3-hydroxybutyrate (P3HB), poly-4-hydroxybutyrate (P4HB), polyhydroxyhexanoate (PHH), polyhydroxyoctanoate (PHO), polyhydroxyvalerate (PHV), or a copolymer thereof. In one aspect, the PHA is PHB.

In some aspects, the nitrogen-fixing microorganism in the biofertilizer comprises bacteria. In some aspects, the nitrogen-fixing microorganism is a PHA-producing bacteria. In some aspects, the nitrogen-fixing microorganism is a PHB-producing bacteria. In some aspects, the nitrogen-fixing microorganism is a PHV-producing bacteria.

In some aspects, the biofertilizer, and methods of producing the same, is described in WO2023/164507A2, which is herein incorporated by reference in its entirety.

In other aspects, the nitrogen-fixing microorganism in the biofertilizer comprises archaea. In other aspects, the nitrogen-fixing microorganism in the biofertilizer comprises fungi.

In one aspect, the nitrogen-fixing microorganism in the biofertilizer is Xanthobacter autotrophicus. In another aspect, the nitrogen-fixing microorganism in the biofertilizer is Ralstonia eutropha. In some aspects, the nitrogen-fixing microorganism in the biofertilizer is Azotobacter vinelandii.

In some aspects, the Xanthobacter autotrophicus comprises Xanthobacter autotrophicus DSM 431, Xanthobacter autotrophicus DSM 432, Xanthobacter autotrophicus DSM 597, Xanthobacter autotrophicus DSM 685, Xanthobacter autotrophicus DSM 1393, Xanthobacter autotrophicus DSM 1618, Xanthobacter autotrophicus DSM 2009, Xanthobacter autotrophicus DSM 2267, Xanthobacter autotrophicus DSM 3874, Xanthobacter autotrophicus CCUG 44692, or any other Xanthobacter autotrophicus strain associated with NCBI Taxonomy ID 280.

In some aspects, the Azotobacter vinelandii comprises Azotobacter vinelandii DSM 2289, Azotobacter vinelandii DSM 279, Azotobacter vinelandii DSM 332, Azotobacter vinelandii DSM 366, Azotobacter vinelandii DSM 382, Azotobacter vinelandii DSM 389, Azotobacter vinelandii DSM 390, Azotobacter vinelandii DSM 395, Azotobacter vinelandii DSM 399, Azotobacter vinelandii DSM 576, Azotobacter vinelandii DSM 720, Azotobacter vinelandii DSM 2290, Azotobacter vinelandii DSM 85, Azotobacter vinelandii DSM 86, Azotobacter vinelandii DSM 87, Azotobacter vinelandii DSM 13529, Azotobacter vinelandii ATCC 9046, or any other Azotobacter vinelandii strain associated with NCBI Taxonomy ID 354.

In some aspects, the microorganism can naturally possess an MISC accumulation pathway.

Some aspects of the present disclosure include a biofertilizer comprising one or more microorganisms. In some aspects, the biofertilizer comprises a nitrogen-fixing microorganism discussed herein. Some aspects of the present disclosure include a biofertilizer comprising more than one microorganisms. In some aspects, the disclosure relates to a biofertilizer comprising a combination of the nitrogen-fixing microorganism with another microorganism. An often used bacterial group in the combination is rhizobacteria, commonly denominated plant growth promoting rhizobacteria (PGPR). PGPR colonizes plant roots and has several functions such as: nitrogen fixation, phosphorus solubilization, phytohormone production (auxins and cytokinins), production of root-growth promoting volatile compounds (e.g., 2-3-butanediol), nitrogen oxidation from organic sources, siderophores production, among others (Bruto, M., Prigent-Combaret, C., Muller, D. et al. Analysis of genes contributing to plant-beneficial functions in plant growth-promoting rhizobacteria and related Proteobacteria. Sci. Rep. 4, 6261, 2014). Exemplary but not-limiting rhizobacteria are Azotobacter spp., Bacillus megaterium, Flavobacterium sp., Acetobacter sp., Azospirillum sp., Bacillus thuringiensis, Bacillus subtillis, Arthrobacter globiformis, Arthrobacter agilis, Nocardia coarallina, Pseudomonas fluorescens, Bacteroides succinogenes, Bacteroides lipolyticum, Kurthis zopfil, Brevibacterium lipolyticum, Aspergillus terreus, Rhizopus arrhizus, Azotobacter chroococcum, Azotobacter paspali, Myrothecium verrucaria, Trichoderma viride, Phanerochaete chrysosporium, Pseudomonas halestorga, Pseudomonas calcis, Pseudomonas gelatic, Pseudomonas marinoglutionosa, Pseudomonas nigriaciens, Brevibacterium stationis, Arthrobacter citreus, Arthrobacter luteus, Arthrobacter simplex, Azosprillum brasilienese, Azosprillum lipoferum, Bacillus brenis, Bacillus macerans, Bacillus pumilus, Bacillus polymyxa, Pseudomonas putida, Streptomycus cellulasae, Streptomycus fradiae, Streptomucus griseoflavus, or Acinetobacter lwoffii.

An exemplary but non-limiting fungi species is Trichoderma sp.

In some aspects, the bacteria is Gluconacetobacter diazotrophicus.

In some aspects, the bacteria is an Azospirillium spp.

In some aspects, the bacteria is Azospirillium brasiliense.

In some aspects, the bacteria is Azospirillum lipoferum (RSAL0111).

In some aspects, the bacteria is a Rhizobium spp.

In some aspects, the bacteria is Rhizobium leguminosarum.

In some aspects, the bacteria is Rhizobium leguminosarum biovar viciae.

In some aspects, the bacteria is Rhizobium japonicum (MCC 0071).

In some aspects, the bacteria is Bradyrhizobium japonicum.

In some aspects, the bacteria is Paenibacillus durus (MCC 0046).

In some aspects, the bacteria is an Azotobacter spp.

In some aspects, the bacteria is Azotobacter chroococum (MCC 0055).

In some aspects, the bacteria is Azotobacter vinelandii.

In some aspects, the bacteria is Azotobacter vinelandii (RSAV006).

In some aspects, the bacteria is Herbaspirillum frisingense (MCC 0052).

In some aspects, the bacteria is Gluconacetobacter diazotrophicus (MCC 0046).

In some aspects, the bacteria is Sinorhizobium meliloti.

In some aspects, the composition comprises a consortium of microorganisms.

In some aspects, the consortium of microorganisms comprises a bacteria, a fungi, a cyanobacteria, or combinations thereof.

In some aspects, the consortium of microorganisms comprises Klebsiella variicola and Kosakonia sacchari.

In some aspects, the consortium of microorganisms comprises Azorhizobium caulinodans, Azoarcus indigens, and Azospirillium brasiliense.

In some aspects, the consortium of microorganisms comprises Rhizobium leguminosarum biovar viciae and Pseudomonas fluorescens.

In some aspects, the consortium of microorganisms comprises Bradyrhizobium japonicum and Penicillium bilaiae.

In some aspects, the consortium of microorganisms comprises Penicillium bilaiae, Bacillus amyloliquefaciens, and Trichoderma virens.

In some aspects, the consortium of microorganisms comprises Penicillium bilaiae, Rhizobium leguminosarum, Bacillus amyloliquefaciens, and Trichoderma virens.

In some aspects, the consortium of microorganisms comprises Bacillus licheniformis, Bacillus megaterium, Bacillus pumilus, Bacillus subtilis, Trichoderma harzianum, Trichoderma reesei, Glomus etunicatum, Glomus intradices, Glomus mosseae, and Glomus aggregatum.

In some aspects, the consortium of microorganisms comprises Bacillus subtilis and Saccharomyces cerevisiae.

In some aspects, the consortium of microorganisms comprises Lactobacillus paracasei, Lactobacillus helveticus, Lactobacillus plantarum, Lactobacillus rhamnosus, Lactococcus lactis, and Candida utilis.

In some aspects, the consortium of composition comprises Xanthobacter autotrophicus and Pseudomonas fluorescens.

In some aspects, the consortium of composition comprises Xanthobacter autotrophicus and Rhodopseudomonas palustris.

In some aspects, the consortium of composition comprises Xanthobacter autotrophicus and Azospirillum lipoferum.

In some aspects, the consortium of composition comprises Xanthobacter autotrophicus and Cupriavidus necator.

### Kits

Certain aspects of the disclosure provide a kit comprising any of the compositions disclosed herein.

Certain aspects of the disclosure provide a kit comprising i) a diluent and ii) a microorganism, wherein the diluent is derived from a plant, yeast, insect, crustacean or algae, and wherein the microorganism is a nitrogen fixing microorganism.

In some aspects, the microorganism is in a dry formulation.

In some aspects, the diluent is in a dry formulation.

In some aspects, the diluent and the microorganism are in a dry formulation.

In some aspects, the composition further comprises one, two, three, four, five, six, or more microorganisms.

In some aspects, the diluent is derived from a plant.

In some aspects, the plant is selected from a group consisting of a coffee plant, a carrot, a potato, a citrus plant, a banana, an alfalfa grass, a tomato, a grape, a rice, a maple tree, and combinations thereof.

In some aspects, the diluent is derived from plant waste or a plant byproduct.

In some aspects, the diluent is derived from a peel of a banana, a rind of a citrus plant, a coffee ground, waste from a grape, a husk of a rice plant, or a maple syrup from a maple tree, or combinations thereof.

In some aspects, the diluent comprises an extract selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least two extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least three extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least four or more extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises an alfalfa extract.

In some aspects, the composition comprises between about 15 g/L and about 50 g/L, between about 15 g/L and about 45 g/L, between about 15 g/L and about 40 g/L, between about 15 g/L and about 35 g/L, between about 15 g/L and about 30 g/L, between about 15 g/L and about 25 g/L, between about 15 g/L and about 20 g/L, between about 20 g/L and about 50 g/L, between about 25 g/L and about 50 g/L, between about 30 g/L and about 50 g/L, between about 35 g/L and about 50 g/L, between about 40 g/L and about 50 g/L, between about 45 g/L and about 50 g/mL, between about 20 g/L and about 40 g/L, between about 21 g/L and about 41 g/L, or between about 25 g/L and about 35 g/L of alfalfa extract.

In some aspects, the composition comprises about 15 g/L, about 20 g/L, about 21 g/L, about 25 g/L, about 30 g/L, about 35 g/L, about 40 g/L, about 45 g/L, or about 50 g/L of alfalfa extract.

In some aspects, the diluent comprises a tomato extract.

In some aspects, the composition comprises between about 0.15 g/L and about 1.5 g/L, between about 0.2 g/L and about 1.5 g/L, between about 0.218 g/L and about 1.5 g/L, between about 0.25 g/L and about 1.5 g/L, between about 0.3 g/L and about 1.5 g/L, between about 0.5 g/L and about 1.5 g/L, between about 0.75 g/L and about 1.5 g/L, between about 1.0 g/L and about 1.5 g/L, between about 1.15 g/L and about 1.5 g/L, between about 0.2 g/L and about 1.15 g/L, between about 0.2 g/L and about 1.0 g/L, between about 0.2 g/L and about 0.75 g/L, between about 0.2 g/L and about 0.5 g/L, between about 0.2 g/L and about 0.4 g/L, between about 0.2 g/L and about 0.3 g/L, between about 0.218 g/L and about 0.3 g/L, or between about 0.2 g/L and about 1.15 g/L of tomato extract.

In some aspects, the composition comprises about 0.15 g/L, about 0.2 g/L, about 0.218 g/L, about 0.25 g/L, about 0.3 g/L, about 0.5 g/L, about 0.75 g/L, about 1.0 g/L, about 1.15 g/L, or about 1.5 g/L of tomato extract.

In some aspects, the diluent comprises an orange extract.

In some aspects, the composition comprises between about 2 g/L and about 4 g/L, between about 2.198 g/L and about 4 g/L, between about 2.25 g/L and about 4 g/L, between about 2.5 g/L and about 4 g/L, between about 3 g/L and about 4 g/L, between about 3.5 g/L and about 4 g/L, between about 3.558 g/L and about 4 g/L, between about 2 g/L and about 3.558 g/L, between about 2 g/L and about 3.5 g/L, between about 2 g/L and about 3 g/L, between about 2 g/L and about 2.5 g/L, or between about 2 g/L and about 2.198 g/L of orange extract.

In some aspects, the composition comprises about 2 g/L, about 2.198 g/L, about 2.25 g/L, about 2.5 g/L, about 3 g/L, about 3.5 g/L, or about 4 g/L of orange extract.

In some aspects, the diluent comprises a rice extract.

In some aspects, the composition comprises between about 5 g/L and about 12 g/L, between about 5 g/L and about 11 g/L, between about 5 g/L and about 10.55 g/L, between about 5 g/L and about 10 g/L, between about 5 g/L and about 9 g/L, between about 5 g/L and about 8 g/L, between about 5 g/L and about 7 g/L, between about 5 g/L and about 6 g/L, between about 5 g/L and about 5.26 g/L, between about 5.26 g/L and about 12 g/L, between about 6 g/L and about 12 g/L, between about 7 g/L and about 12 g/L, between about 8 g/L and about 12 g/L, between about 9 g/L and about 12 g/L, between about 10 g/L and about 12 g/L, between about 10.55 g/L and about 12 g/L, or between about 11 g/L and about 12 g/L of rice extract.

In some aspects, the composition comprises about 5 g/L, about 5.26 g/L, about 7 g/L, about 8 g/L, about 9 g/L, about 10 g/L, about 10.55 g/L, about 11 g/L, or about 12 g/L of rice extract.

In some aspects, the diluent is derived from an algae.

In some aspects, the diluent comprises a Chlorella extract.

In some aspects, the composition comprises between about 1 g/L and about 5 g/L, between about 2 g/L and about 5 g/L, between about 3 g/L and about 5 g/L, between about 4 g/L and about 5 g/L, between about 1 g/L and about 4 g/L, between about 1 g/L and about 3 g/L, between about 1 g/L and about 2 g/L, between about 2 g/L and about 4 g/L of Chlorella extract.

In some aspects, the composition comprises about 1 g/L, about 2 g/L, about 3 g/L, about 4 g/L or about 5 g/L of Chlorella extract.

In some aspects, the diluent is derived from a yeast.

In some aspects, the yeast is a brewing yeast.

In some aspects, the yeast is a spent brewing yeast.

In some aspects, the diluent comprises a yeast extract.

In some aspects, the composition comprises between about 1 g/L and about 5 g/L, between about 2 g/L and about 5 g/L, between about 3 g/L and about 5 g/L, between about 4 g/L and about 5 g/L, between about 1 g/L and about 4 g/L, between about 1 g/L and about 3 g/L, between about 1 g/L and about 2 g/L, between about 2 g/L and about 4 g/L of yeast extract.

In some aspects, the composition comprises about 1 g/L, about 2 g/L, about 3 g/L, about 4 g/L or about 5 g/L of yeast extract.

In some aspects, the diluent is derived from an insect.

In some aspects, the insect is a bee.

In some aspects, the diluent is derived from honey or bee's wax.

In some aspects, the diluent is derived from the chitosan of an insect.

In some aspects, the composition comprises between about 25 mg/L and about 600 mg/L, between about 50 mg/L and about 600 mg/L, between about 75 mg/L and about 600 mg/L, between about 100 mg/L and about 600 mg/L, between about 200 mg/L and about 600 mg/L, between about 300 mg/L and about 600 mg/L, between about 400 mg/L and about 600 mg/L, between about 500 mg/L and about 600 mg/L, between about 50 mg/L and about 500 mg/L, between about 25 mg/L and about 500 mg/L, between about 25 mg/L and about 400 mg/L, between about 25 mg/L and about 300 mg/L, between about 25 mg/L and about 200 mg/L, between about 25 mg/L and about 100 mg/L, between about 25 mg/L and about 75 mg/L, or between about 25 mg/L and about 50 mg/L of chitosan.

In some aspects, the composition comprises about 25 mg/L, about 50 mg/L, about 75 mg/L, about 100 mg/L, about 200 mg/L, about 300 mg/L, about 400 mg/L, about 500 mg/L, or about 600 mg/L of chitosan.

In some aspects, the diluent comprises a Chlorella extract and a yeast extract.

In some aspects, the microorganism is a bacteria.

In some aspects, the bacteria is selected from the group consisting of Acidiphilium multivorum, Acidiphilium species, Alcaligenes paradoxus, Alcaligenes species, Arthrobacter species, Azoarcus indigens, Azohydromonas australica, Azohydromonas lata, Azohydromonas species, Azorhizobium caulinodans, Azospirillium brasiliense, Azospirillium spp., Azospirillum amazonsense, Azospirillum lipoferum, Azospirillum lipoferum (RSAL0111), Azospirillum species, Azospirillum thiophilum, Azotobacter chroococum (MCC 0055), Azotobacter spp., Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beggiatoa species, Beijerinckia mobilis, Beijerinckia species, Bradyrhizobium elnakii, Bradyrhizobium japonicum, Bradyrhizobium japonicum (strain USDA 122), Bradyrhizobium species, Burkholderia species, Burkholderia vietnameiensis, Cupriavidus necator, Cupriavidus species, Cyanobacteria species, Derxia gummosa, Derxia species, Gluconacetobacter diazotrophicus, Gluconacetobacter diazotrophicus (MCC 0046), Herbaspirillum autrotrophicum, Herbaspirillum frisingense (MCC 0052), Herbaspirillum species, Hydrogenophaga pseudoflava, Hydrogenophaga species, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Mesorhizobium species, Methylibium petroleiphilum, Methylibium species, Methylocapsa aurea, Methylocapsa species, Methyloferula species, Methyloferula stellate, Methyloversatilis species, Methyloversatilis universalis, Microcyclus aquaticus, Microcyclus ebruneus, Microcyclus species, Nitrosococcus oceani, Nitrosococcus species, Nitrosomonas communis, Nitrospirillum amazonense, Nocardia autotrophica, Nocardia opaca, Nocardia species, Oligotropha carboxidovorans, Oligotropha species, Paenibacillus durus (MCC 0046), Pannonibacter phragmitetus, Pannonibacter species, Paracoccus denitrificans, Paracoccus pantrophus, Paracoccus species, Paracoccus yeei, Pelagibaca bermudensis, Pelagibaca species, Pseudomonas facilis, Pseudomonas fluorescens, Pseudomonas species, Pseudooceanicola atlanticus, Pseudooceanicola species, Ralstonia eutropha, Ralstonia species, Renobacter species, Renobacter vacuolatum, Rhizobium gallicum, Rhizobium japonicum, Rhizobium japonicum (MCC 0071), Rhizobium leguminosarum, Rhizobium leguminosarum biovar viciae, Rhizobium species, Rhizobium spp., Rhodobacter capsulatus, Rhodobacter species, Rhodobacter sphaeroides, Rhodomicrobium species, Rhodomicrobium vannielii, Rhodopseudomonas palustris, Rubrivivax gelatinosus, Rubrivivax species, Salipiger mucosus, Salipiger species, Sinorhizobium americanum, Sinorhizobium fredii, Sinorhizobium meliloti, Sinorhizobium species, Skermanella species, Skermanella stibiiresistens, Stappia aggregate, Stappia species, Thauera humireducens, Thauera species, Variovorax paradoxus, Variovorax species, Xanthobacter autotrophicus, Xanthobacter species, and combinations thereof.

In some aspects, the bacteria is Xanthobacter autotrophicus.

In some aspects, the nitrogen fixing microorganism is a fungi.

In some aspects, the fungi is selected from the group consisting of Glomus aggregatum, Glomus intraradices, Glomus mosseae, Glomus etunicatum, Trichoderma reesei, Candida utilis, Penicillium bilaiae, Saccharomyces cerevisiae, Trichoderma harzianum, Trichoderma virens, and combinations thereof.

In some aspects, the kit comprises at least about 0.1% w/w, at least about 1% w/w, at least about 10% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of the diluent (i.e., as measured after the diluent and microorganism have been combined to form a composition).

In some aspects, the kit comprises at least 0.1% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w of the diluent.

In some aspects, the kit comprises about 0.1% w/w, about 1% w/w, about 10% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, about 95% w/w, or about 99% w/w of the diluent.

In some aspects, the kit comprises between about 0.1% w/w and about 99% w/w, between about 1% w/w and about 99% w/w, between about 5% w/w and about 99% w/w, between about 10% w/w and about 99% w/w, between about 20% w/w and about 99% w/w, between about 30% w/w and about 99% w/w, between about 40% w/w and about 99% w/w, between about 50% w/w and about 99% w/w, between about 60% w/w and about 99% w/w, between about 70% w/w and about 99% w/w, between about 75% w/w and about 99% w/w, between about 80% w/w and about 99% w/w, between about 90% w/w and about 99% w/w, between about 95% w/w and about 99% w/w, between about 0.1% w/w and about 90% w/w, between about 1% w/w and about 90% w/w, between about 0.1% w/w and about 80% w/w, between about 1% w/w and about 80% w/w, between about 0.1% w/w and about 70% w/w, between about 1% w/w and about 70% w/w, between about 0.1% w/w and about 60% w/w, between about 1% w/w and about 60% w/w, between about 0.1% w/w and about 50% w/w, between about 1% w/w and about 50% w/w, between about 0.1% w/w and about 40% w/w, between about 1% w/w and about 40% w/w, between about 0.1% w/w and about 30% w/w, between about 1% w/w and about 30% w/w, between about 0.1% w/w and about 20% w/w, between about 1% w/w and about 20% w/w, between about 0.1% w/w and about 10% w/w, between about 1% w/w and about 10% w/w, between about 35% w/w and about 65% w/w, between about 40% w/w and about 65% w/w, between about 45% w/w and about 65% w/w, between about 50% w/w and about 65% w/w, between about 55% w/w and about 65% w/w, between about 60% w/w and about 65% w/w, between about 35% w/w and about 60% w/w, between about 35% w/w and about 55% w/w, between about 35% w/w and about 50% w/w, between about 35% w/w and about 45% w/w, between about 35% w/w and about 40% w/w, between about 45% w/w and about 55% w/w, between about 40% w/w and about 60% w/w, or between about 40% w/w and about 50% w/w of the diluent.

In some aspects, the kit comprises between 0.1% w/w and 99% w/w, between 1% w/w and 99% w/w, between 5% w/w and 99% w/w, between 10% w/w and 99% w/w, between 20% w/w and 99% w/w, between 30% w/w and 99% w/w, between 40% w/w and 99% w/w, between 50% w/w and 99% w/w, between 60% w/w and 99% w/w, between 70% w/w and 99% w/w, between 75% w/w and 99% w/w, between 80% w/w and 99% w/w, between 90% w/w and 99% w/w, between 95% w/w and 99% w/w, between 0.1% w/w and 90% w/w, between 1% w/w and 90% w/w, between 0.1% w/w and 80% w/w, between 10% w/w and 80% w/w, between 0.10% w/w and 70% w/w, between 1% w/w and 70% w/w, between 0.1% w/w and 60% w/w, between 1% w/w and 60% w/w, between 0.1% w/w and 50% w/w, between 1% w/w and 50% w/w, between 0.1% w/w and 40% w/w, between 1% w/w and 40% w/w, between 0.1% w/w and 30% w/w, between 1% w/w and 30% w/w, between 0.1% w/w and 20% w/w, between 1% w/w and 20% w/w, between 0.1% w/w and 10% w/w, between 1% w/w and 10% w/w, between 35% w/w and 65% w/w, between 40% w/w and 65% w/w, between 45% w/w and 65% w/w, between 50% w/w and 65% w/w, between 55% w/w and 65% w/w, between 60% w/w and 65% w/w, between 35% w/w and 60% w/w, between 35% w/w and 55% w/w, between 35% w/w and 50% w/w, between 35% w/w and 45% w/w, between 35% w/w and 40% w/w, between 45% w/w and 55% w/w, between 40% w/w and 60% w/w, or between 40% w/w and 50% w/w of the diluent.

In some aspects, the kit comprises at least about 0.1% w/w, at least about 1% w/w, at least about 10% w/w, at least about 20% w/w, at least about 25% w/w, at least about 30% w/w, at least about 35% w/w, at least about 40% w/w, at least about 45% w/w, at least about 50% w/w, at least about 55% w/w, at least about 60% w/w, at least about 65% w/w, at least about 70% w/w, at least about 75% w/w, at least about 80% w/w, at least about 85% w/w, at least about 90% w/w, at least about 95% w/w, or at least about 99% w/w of the microorganism. (i.e., as measured after the diluent and microorganism have been combined to form a composition).

In some aspects, the kit comprises at least 0.1% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w of the microorganism.

In some aspects, the kit comprises about 0.1% w/w, about 1% w/w, about 10% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, about 95% w/w, or about 99% w/w of the microorganism.

In some aspects, the kit comprises between about 0.1% w/w and about 99% w/w, between about 1% w/w and about 99% w/w, between about 5% w/w and about 99% w/w, between about 0% w/w and about 99% w/w, between about 20% w/w and about 99% w/w, between about 30% w/w and about 99% w/w, between about 40% w/w and about 99% w/w, between about 50% w/w and about 99% w/w, between about 60% w/w and about 99% w/w, between about 70% w/w and about 99% w/w, between about 750% w/w and about 99% w/w, between about 80% w/w and about 99% w/w, between about 90% w/w and about 99% w/w, between about 95% w/w and about 99% w/w, between about 0.1% w/w and about 90% w/w, between about 1% w/w and about 90% w/w, between about 0.1% w/w and about 80% w/w, between about 1% w/w and about 80% w/w, between about 0.1% w/w and about 70% w/w, between about 1% w/w and about 70% w/w, between about 0.1% w/w and about 60% w/w, between about 1% w/w and about 60% w/w, between about 0.1% w/w and about 50% w/w, between about 1% w/w and about 50% w/w, between about 0.1% w/w and about 40% w/w, between about 1% w/w and about 40% w/w, between about 0.1% w/w and about 30% w/w, between about 1% w/w and about 30% w/w, between about 0.1% w/w and about 20% w/w, between about 1% w/w and about 20% w/w, between about 0.1% w/w and about 5% w/w, between about 1% w/w and about 10% w/w, between about 35% w/w and about 65% w/w, between about 40% w/w and about 65% w/w, between about 45% w/w and about 65% w/w, between about 50% w/w and about 65% w/w, between about 55% w/w and about 65% w/w, between about 60% w/w and about 65% w/w, between about 35% w/w and about 60% w/w, between about 35% w/w and about 55% w/w, between about 35% w/w and about 50% w/w, between about 35% w/w and about 45% w/w, between about 35% w/w and about 40% w/w, between about 45% w/w and about 55% w/w, between about 40% w/w and about 60% w/w, or between about 40% w/w and about 50% w/w of the microorganism.

In some aspects, the kit comprises between 0.1% w/w and 99% w/w, between 1% w/w and 99% w/w, between 5% w/w and 99% w/w, between 10% w/w and 99% w/w, between 20% w/w and 99% w/w, between 30% w/w and 99% w/w, between 40% w/w and 99% w/w, between 50% w/w and 99% w/w, between 60% w/w and 99% w/w, between 70% w/w and 99% w/w, between 75% w/w and 99% w/w, between 80% w/w and 99% w/w, between 90% w/w and 99% w/w, between 95% w/w and 99% w/w, between 0.1% w/w and 90% w/w, between 1% w/w and 90% w/w, between 0.1% w/w and 80% w/w, between 1% w/w and 80% w/w, between 0.1% w/w and 70% w/w, between 1% w/w and 70% w/w, between 0.1% w/w and 60% w/w, between 1% w/w and 60% w/w, between 0.1% w/w and 50% w/w, between 1% w/w and 50% w/w, between 0.1% w/w and 40% w/w, between 1% w/w and 40% w/w, between 0.1% w/w and 30% w/w, between 1% w/w and 30% w/w, between 0.1% w/w and 20% w/w, between 1% w/w and 20% w/w, between 0.1% w/w and 10% w/w, between 1% w/w and 10% w/w, between 35% w/w and 65% w/w, between 40% w/w and 65% w/w, between 45% w/w and 65% w/w, between 50% w/w and 65% w/w, between 55% w/w and 65% w/w, between 60% w/w and 65% w/w, between 35% w/w and 60% w/w, between 35% w/w and 55% w/w, between 35% w/w and 50% w/w, between 35% w/w and 45% w/w, between 35% w/w and 40% w/w, between 45% w/w and 55% w/w, between 40% w/w and 60% w/w, or between 40% w/w and 50% w/w of the microorganism.

In some aspects, the kit comprises between about 1×10⁵ CFU/mL and about 1×10¹⁰ CFU/mL of the microbial cells.

In some aspects, the kit comprises between about 1×10⁹ CFU/mL and about 4×10⁹ CFU/mL of the microbial cells.

In some aspects, the kit comprises between about 1×10⁵ CFU/mL and about 1×10¹⁰ CFU/mL of the microbial cells, between about 1×10⁵ CFU/mL and about 1×10⁹ CFU/mL of the microbial cells, between about 1×10⁵ CFU/mL and about 1×10⁸ CFU/mL of the microbial cells, between about 1×10⁵ CFU/mL and about 1×10⁷ CFU/mL of the microbial cells, between about 1×10⁵ CFU/mL and about 1×10⁶ CFU/mL of the microbial cells, between about 1×10⁶ CFU/mL and about 1×10¹⁰ CFU/mL of the microbial cells, between about 1×10⁷ CFU/mL and about 1×10¹⁰ CFU/mL of the microbial cells, between about 1×10⁸ CFU/mL and about 1×10¹⁰ CFU/mL of the microbial cells, between about 1×10⁹ CFU/mL and about 1×10¹⁰ CFU/mL of the microbial cells, between about 1×10⁷ CFU/mL and about 1×10⁹ CFU/mL of the microbial cells, between about 1×10⁹ CFU/mL and about 4×10⁹ CFU/mL of the microbial cells, between about 2×10⁹ CFU/mL and about 4×10⁹ CFU/mL of the microbial cells, between about 3×10⁹ CFU/mL and about 4×10⁹ CFU/mL of the microbial cells, between about 1×10⁹ CFU/mL and about 3×10⁹ CFU/mL of the microbial cells, between about 1×10⁹ CFU/mL and about 2×10⁹ CFU/mL of the microbial cells, or between about 2×10⁹ CFU/mL and about 3×10⁹ CFU/mL of the microbial cells.

In some aspects, the kit comprises at least about 1×10⁹ CFU/mL of the microbial cells.

In some aspects, the kit comprises at least about 1×10⁵ CFU/mL, at least about 1×10⁶ CFU/mL, at least about 1×10⁷ CFU/mL, at least about 1×10⁸ CFU/mL, at least about 1×10⁹ CFU/mL, at least about 2×10⁹ CFU/mL, at least about 3×10⁹ CFU/mL, at least about 4×10⁹ CFU/mL, or at least about 1×10¹⁰ CFU/mL of the microbial cells.

In some aspects, the kit comprises at least 1×10⁵ CFU/mL, at least 1×10⁶ CFU/mL, at least 1×10⁷ CFU/mL, at least 1×10⁸ CFU/mL, at least 1×10⁹ CFU/mL, at least 2×10⁹ CFU/mL, at least 3×10⁹ CFU/mL, at least 4×10⁹ CFU/mL, or at least 1×10¹⁰ CFU/mL of the microbial cells.

In some aspects, the kit comprises about 1×10⁵ CFU/mL, about 1×10⁶ CFU/mL, about 1×10⁷ CFU/mL, about 1×10⁸ CFU/mL, about 1×10⁹ CFU/mL, about 2×10⁹ CFU/mL, about 3×10⁹ CFU/mL, about 4×10⁹ CFU/mL, or about 1×10¹⁰ CFU/mL of the microbial cells.

In some aspects, the kit comprises Xanthobacter autotrophicus and Pseudomonas fluorescens.

In some aspects, the kit comprises Xanthobacter autotrophicus and Rhodopseudomonas palustris.

In some aspects, the kit comprises Xanthobacter autotrophicus and Azospirillum lipoferum.

In some aspects, the kit comprises Xanthobacter autotrophicus and Cupriavidus necator.

In some aspects, the kit comprises i) about 1×10⁵ CFU/mL, about 1×10⁶ CFU/mL, about 1×10⁷ CFU/mL, about 1×10⁸ CFU/mL, about 5×10⁸ CFU/mL, about 1×10⁹ CFU/mL, about 2×10⁹ CFU/mL, about 3×10⁹ CFU/mL, about 4×10⁹ CFU/mL, or about 1×10¹⁰ CFU/mL of a first microbial cell; and ii) i) about 1×10⁵ CFU/mL, about 1×10⁶ CFU/mL, about 1×10⁷ CFU/mL, about 1×10⁸ CFU/mL, about 5×10⁸ CFU/mL, about 1×10⁹ CFU/mL, about 2×10⁹ CFU/mL, about 3×10⁹ CFU/mL, about 4×10⁹ CFU/mL, or about 1×10¹⁰ CFU/mL of a second microbial cell.

In some aspects, the kit comprises i) about 5×10⁸ CFU/mL of Xanthobacter autotrophicus and ii) about 5×10⁸ CFU/mL of a second microbial cell selected from Pseudomonas fluorescens, Rhodopseudomonas palustris, Azospirillum lipoferum, or Cupriavidus necator.

### METHODS OF USE

### Nitrogen Utilization Efficiency

Certain aspects of the disclosure provide a method of increasing nitrogen utilization efficiency of a plant, comprising administering to the plant any of the compositions, diluents, or microorganisms disclosed herein.

In some aspects, the method comprises combining the components of the kit to form a composition, then administering to the plant said composition.

In some aspects, the plant is wheat (Triticum aestivum), rice (Oryza sativa), maize (Zea mays), barley (Hordeum vulgare), cotton (Gossypium spp.), sugarcane (Saccharum officinarum), tobacco (Nicotiana tabacum), soybean (Glycine max), sunflower (Helianthus annuus), mustard (Brassica juncea), groundnut (Arachis hypogaea), hemp (Cannabis sativa), flax (Linum usitatissimum), black pepper (Piper nigrum), potatoes (Solanum tuberosum), tomatoes (Solanum lycopersicum), onions (Allium cepa), coffee (Coffea spp.), tea (Camellia sinensis), cocoa (Theobroma cacao), hops (Humulus lupulus), lettuce (Lactuca sativa), garlic (Allium sativum), celery (Apium graveolens), pepper (Capsicum spp.), broccoli (Brassica oleracea var. italica), cabbage (Brassica oleracea var. capitata), canola (Brassica napus), cauliflower (Brassica oleracea var. botrytis), orange (Citrus sinensis), lemon (Citrus limon), tangerine (Citrus reticulata), cucumber (Cucumis sativus), melon (Cucumis melo), squash (Cucurbita spp.), strawberry (Fragaria ananassa), alfalfa (Medicago sativa), palm (Arecaceae family), pistachio (Pistacia vera), stone fruits (Prunus spp.), raspberry (Rubus idaeus), turf grass (Poaceae family), blueberry (Vaccinium spp.), or grape (Vitis vinfera).

In some aspects, the plant is selected from Triticum spp., Hordeum spp., Gossypium spp., Saccharum spp., Nicotiana spp., Glycine spp., Helianthus spp., Brassica spp., Arachis spp., Linum spp., Piper spp., Coffea spp., Camellia spp., Theobroma spp., Humulus spp., Lactuca spp., Allium spp., Appium graveolens, Brassica spp., Cannabis spp., Capsicum spp., Citrus spp., Cucumis spp., Cucurbita spp., Fragaria spp., Gossypium spp., Hordeum spp., Medicago spp., Oryza spp., Palma spp., Pasticia spp., Prunus spp., Rudus spp., Succharum spp., Solanum spp., Triticum spp., Vaccinium spp., Vitis spp., or Zea spp.

Nitrogen utilization efficiency (NUE) is an established metric used to benchmark N management. There are a number of different measures for NUE.

In some aspects, nitrogen utilization efficiency is measured as: (Yield Nt)/(fertilizer N), wherein Yield Nt is the amount of nitrogen incorporated a crop, and fertilizer N is the amount of fertilizer nitrogen added to the crop.

In some aspects, the NUE is about 40%, about 45%, about 50%, about 55%, or about 60%.

In some aspects, the NUE is 40%, 45%, 50%, 55%, or 60%.

In some aspects, the NUE is between about 40% to about 60%, about 45% to about 60%, about 50% to about 60%, about 55% to about 60%, about 40% to about 55%, about 40% to about 50%, about 40% to about 45%, or about 45% to about 55%.

### Increased Biomass, Fruit Quality, Growth Rate, Yield of Plant, Lateral Root Density

Certain aspects of the disclosure provide a method of increasing biomass, fruit quality, growth rate, lateral root density, or yield of a plant comprising administering to the plant any of the compositions, diluents, or microorganisms disclosed herein.

In some aspects, the method comprises combining the components of the kit to form a composition, then administering to the plant said composition.

In some aspects, the biomass is measured via aboveground fresh weight.

In some aspects, the biomass is measured via aboveground dry weight.

### Resistance to Growth Disease

Certain aspects of the disclosure provide a method of increasing resistance to growth disease in a plant comprising administering to the plant any of the compositions, diluents, or microorganisms disclosed herein.

In some aspects, the method comprises combining the components of the kit to form a composition, then administering to the plant said composition.

In some aspects, the growth disease is a fungal disease.

In some aspects, the fungal disease is powdery mildew, downy mildew, or blight.

In some aspects, the growth disease is tip burn.

### Resistance to Chemical Over Application

Certain aspects of the disclosure provide a method of increasing resistance to chemical over application in a plant comprising administering to the plant any of the compositions, diluents, or microorganisms disclosed herein.

In some aspects, the method comprises combining the components of the kit to form a composition, then administering to the plant said composition.

In some aspects, the composition comprises between about 1×10⁵ CFU and about 1×10¹⁰ CFU of the microbial cells.

In some aspects, the composition comprises between about 1×10⁹ CFU and about 4×10⁹ CFU of the microbial cells.

In some aspects, the composition comprises between about 1×10⁵ CFU and about 1×10¹⁰ CFU of the microbial cells, between about 1×10⁵ CFU and about 1×10⁹ CFU of the microbial cells, between about 1×10⁵ CFU and about 1×10⁸ CFU of the microbial cells, between about 1×10⁵ CFU and about 1×10⁷ CFU of the microbial cells, between about 1×10⁵ CFU and about 1×10⁶ CFU of the microbial cells, between about 1×10⁶ CFU and about 1×10¹⁰ CFU of the microbial cells, between about 1×10⁷ CFU and about 1×10¹⁰ CFU of the microbial cells, between about 1×10⁸ CFU and about 1×10¹⁰ CFU of the microbial cells, between about 1×10⁹ CFU and about 1×10¹⁰ CFU of the microbial cells, between about 1×10⁷ CFU and about 1×10⁹ CFU of the microbial cells, between about 1×10⁹ CFU and about 4×10⁹ CFU of the microbial cells, between about 2×10⁹ CFU and about 4×10⁹ CFU of the microbial cells, between about 3×10⁹ CFU and about 4×10⁹ CFU of the microbial cells, between about 1×10⁹ CFU and about 3×10⁹ CFU of the microbial cells, between about 1×10⁹ CFU and about 2×10⁹ CFU of the microbial cells, or between about 2×10⁹ CFU and about 3×10⁹ CFU of the microbial cells.

In some aspects, the composition comprises at least about 1×10⁹ CFU of the microbial cells.

In some aspects, the composition comprises at least about 1×10⁵ CFU, at least about 1×10⁶ CFU, at least about 1×10⁷ CFU, at least about 1×10⁸ CFU, at least about 1×10⁹ CFU, at least about 2×10⁹ CFU, at least about 3×10⁹ CFU, at least about 4×10⁹ CFU, or at least about 1×10¹⁰ CFU of the microbial cells.

In some aspects, the composition comprises at least 1×10⁵ CFU, at least 1×10⁶ CFU, at least 1×10⁷ CFU, at least 1×10⁸ CFU, at least 1×10⁹ CFU, at least 2×10⁹ CFU, at least 3×10⁹ CFU, at least 4×10⁹ CFU, or at least 1×10¹⁰ CFU of the microbial cells.

In some aspects, the composition comprises about 1×10⁵ CFU, about 1×10⁶ CFU, about 1×10⁷ CFU, about 1×10⁸ CFU, about 1×10⁹ CFU, about 2×10⁹ CFU, about 3×10⁹ CFU, about 4×10⁹ CFU, or about 1×10¹⁰ CFU of the microbial cells.

### Improving the Efficacy of a Microorganism

Certain aspects of the disclosure provide a method of improving the efficacy of a microorganism in a biofertilizer comprising contacting the microorganism with a diluent, wherein the diluent is derived from a plant, yeast, insect, or algae, wherein the microorganism is a nitrogen-fixing microorganism.

In some aspects, the diluent is derived from a plant.

In some aspects, the plant is selected from a group consisting of a coffee plant, a carrot, a potato, an orange, a banana, an alfalfa grass, a tomato, a grape, a rice, or a maple tree.

In some aspects, the diluent is derived from a peel of a banana, a peel of an orange, a coffee ground, a husk of a rice plant, or a syrup from a maple tree.

In some aspects, the diluent is derived from an algae.

In some aspects, the diluent is derived from a yeast.

In some aspects, the yeast is a brewing yeast.

In some aspects, the yeast is a spent brewing yeast.

In some aspects, the diluent is derived from an insect.

In some aspects, the insect is a bee.

In some aspects, the diluent is derived from honey or bee's wax.

In some aspects, the diluent comprises an extract selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least two extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least three extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

In some aspects, the diluent comprises at least four or more extracts selected from an alfalfa extract, a coffee extract, a carrot extract, a potato extract, a tomato extract, an orange extract, a banana extract, a grape extract, a rice extract, a maple syrup, an algae extract, a yeast extract, an insect extract (e.g., chitosan), or a crustacean extract (e.g., chitosan).

### EXAMPLES

### Example 1. Minimal CFUs Required with Biofertilizer for Lettuce

The purpose of this experiment was to observe a dose response in lettuce when applying a biofertilizer comprising a microorganism (e.g., Xanthobacter autotrophicus with high Polyhydroxybutyrate (PHB)) with an optimal application method. This experiment determined a minimal threshold at which the biofertilizer confers a benefit to lettuce, and the effect that increasing the amount of the biofertilizer has on plant growth.

Treatments were included to measure an optimal application rate with and without the addition of Diluent #1.

The objective of this experiment was to observe a dose response in lettuce plants. Total CFUs ranged from 1.0E9 to 4.0E10 with the intent of finding a lower threshold at which the biofertilizer could be dosed to provide a significant benefit.

Furthermore, there were treatments diluted 10× in diluent #1. This added an additional element to the optimal application rate as it was determined if the threshold number of CFUs for observing a replacement effect can be further lowered when the product is supplemented with Diluent #1.

### Experimental Design

120 plants were used, with 10 treatments applied to 6 plants in duplicate, as described in Table 1. Microorganisms were applied by either root dipping or pipette additions.

For root dipping, the following steps were performed:
a) 12 seedlings were taken up at a time and placed in weigh-boat, or some other small container.
b) Microbial product was diluted aseptically to desired density in water, in separate sterile 50 mL tube. Product was mixed/vortexed while diluting. Total volume should be equal to number of plants ×3 (in mL).
(c) 3 mL of dilute microbial product was added per seedling to weigh boat.
(d) The seedling and microbial product soaked and sat for 5 minutes.
(e) The seedlings were transplanted as normal in randomized pattern into pots.

For pipetting, the following steps were performed:
(a) Desired application volume and density was determined as part of experimental design.
(b) Microbial product was aseptically diluted to desired density in water, in separate sterile 50 mL tube. Product was mixed/vortexed while diluting. Total volume should be equal to number of plants ×3 (in mL).
(c) Sealed container of microbial product was transferred to grow box.
(d) Desired volume was pipetted to the base of each plant, being sure to mix diluted sample throughout. Directly touching plant/dirt with pipette tip was avoided when going between diluted mixture and application. Note: excessive watering of pots was avoided after dosing plants with microbial product.

TABLE 1
Treatment Groups

| | | | | Week 1 | | | Week 2 | | | Week 3 | | | Week 4 | | |
|---|---|---|---|---|---|---|---|---|---|---|---|---|---|---|---|
| | | | | Day 0 | | | Day 7 | | | Day 14 | | | Day 21 | | |
| | | | | CFU | ml | Day | CFU | ml | Day | CFU | ml | Day | CFU | ml | Day |
| | Total N | | | Bio- | Bio- | 1 | Bio- | Bio- | 8 | Bio- | Bio- | 15 | Bio- | Bio- | 22 |
| Treat- | | lb | % | fertil- | fertil- | lb | fertil- | fertil- | lb | fertil- | fertil- | lb | fertil- | fertil- | 1b |
| ments | Type | N/ac | GSP | izer | izer | N/ac | izer | izer | N/ac | izer | izer | N/ac | izer | izer | N/ac |

Low N UTC

| | | | | | | | |
|---|---|---|---|---|---|---|---|
| Low N UTC | UAN-32 | 38.4 | 80% | 9.6 | 9.6 | 9.6 | 9.6 |

4.33e10 product density

| | | | | | | | | | | | | | | | |
|---|---|---|---|---|---|---|---|---|---|---|---|---|---|---|---|
| 1.00E+09 | UAN-32 | 38.4 | 80% | 5.00E+08 | 0.012 | 9.6 | 2.00E+08 | 0.005 | 9.6 | 2.00E+08 | 0.005 | 9.6 | 2.00E+08 | 0.005 | 9.6 |
| 4.00E+09 | UAN-32 | 38.4 | 80% | 2.00E+09 | 0.046 | 9.6 | 8.00E+08 | 0.018 | 9.6 | 8.00E+08 | 0.018 | 9.6 | 8.00E+08 | 0.018 | 9.6 |
| 1.00E+10 | UAN-32 | 38.4 | 80% | 5.00E+09 | 0.115 | 9.6 | 2.00E+09 | 0.046 | 9.6 | 2.00E+09 | 0.046 | 9.6 | 2.00E+09 | 0.046 | 9.6 |
| 4.00E+10 | UAN-32 | 38.4 | 80% | 2.00E+10 | 0.462 | 9.6 | 8.00E+09 | 0.185 | 9.6 | 8.00E+09 | 0.185 | 9.6 | 8.00E+09 | 0.185 | 9.6 |

4.33e9 product density

| | | | | | | | | | | | | | | | |
|---|---|---|---|---|---|---|---|---|---|---|---|---|---|---|---|
| 1.00E+09 | UAN-32 | 38.4 | 80% | 5.00E+07 | 0.012 | 9.6 | 2.00E+08 | 0.005 | 9.6 | 2.00E+08 | 0.005 | 9.6 | 2.00E+08 | 0.005 | 9.6 |
| 4.00E+09 | UAN-32 | 38.4 | 80% | 2.00E+09 | 0.046 | 9.6 | 8.00E+08 | 0.018 | 9.6 | 8.00E+08 | 0.018 | 9.6 | 8.00E+08 | 0.018 | 9.6 |
| 1.00E+10 | UAN-32 | 38.4 | 80% | 5.00E+09 | 0.115 | 9.6 | 2.00E+09 | 0.046 | 9.6 | 2.00E+09 | 0.046 | 9.6 | 2.00E+09 | 0.046 | 9.6 |
| 4.00E+10 | UAN-32 | 38.4 | 80% | 2.00E+10 | 0.462 | 9.6 | 8.00E+09 | 0.185 | 9.6 | 8.00E+09 | 0.185 | 9.6 | 8.00E+09 | 0.185 | 9.6 |

High N UTC

| | | | | | | | |
|---|---|---|---|---|---|---|---|
| High N UTC | UAN-32 | 48 | 100% | 12 | 12 | 12 | 12 |

Biofertilizer/Diluent #1 was dosed as described in Table 2:

TABLE 2
Biofertilizer/Diluent #1 dosage

| | | Per Plant (mL) | | Per Treatment (mL) | |
|---|---|---|---|---|---|
| | | Volume | Volume | Volume | Volume |
| | Dose | Biofertilizer | Water | Biofertilizer | Water |
| Biofertilizer | 2.00E+08 | 0.005 | 0.995 | 0.069 | 14.931 |
| | 5.00E+08 | 0.012 | 0.988 | 0.173 | 14.827 |
| | 8.00E+08 | 0.018 | 0.982 | 0.277 | 14.723 |
| | 2.00E+09 | 0.046 | 0.954 | 0.693 | 14.307 |
| | 5.00E+09 | 0.115 | 0.885 | 1.732 | 13.268 |
| | 8.00E+09 | 0.185 | 0.815 | 2.771 | 12.229 |
| | 2.00E+10 | 0.462 | 0.538 | 6.928 | 8.072 |
| Biofertilizer 10x | 2.00E+07 | 0.005 | 0.995 | 0.069 | 14.931 |
| dilution into | 5.00E+07 | 0.012 | 0.988 | 0.173 | 14.827 |
| D#1 | 8.00E+07 | 0.018 | 0.982 | 0.277 | 14.723 |
| | 2.00E+08 | 0.046 | 0.954 | 0.693 | 14.307 |
| | 5.00E+08 | 0.115 | 0.885 | 1.732 | 13.268 |
| | 8.00E+08 | 0.185 | 0.815 | 2.771 | 12.229 |
| | 2.00E+09 | 0.462 | 0.538 | 6.928 | 8.072 |

At the beginning of the experiment, a 1:10 dilution of the biofertilizer was made by mixing 2 mL of the biofertilizer into 18 mL of Diluent #1. This was kept sterile in a refrigerator through the entirety of the experiment. The Diluent #1 was steeped at 55° C. and made at a concentration of 21.13 g/L.

Nitrogen Fertilizer Treatments were dosed at 50 mL to the base of each plant. Calculations for UAN-32 additions to 10000 Hoagland's solution without N are listed in Table 3.

Tables 4-7 describe the designs for Replacement Effect (Table 4), Replacement and Boosting Effect (Table 5), Application Rate Comparison (Table 6), and Diluent optimization—final cell density (Table 7).

TABLE 3
Fertilizer Treatment

| | | | | | | UAN32: | Per Plant: | Fertilizer | mL |
|---|---|---|---|---|---|---|---|---|---|
| N Dose | N Dose | Pot | Total N | UAN32 | UAN32 | N | UAN32 | Volume | UAN/L |
| (lb | (mg | size | per pot | (Nitrogen) | Density | concentration | Application | Needed | Hoagland |
| N/acre) | N/m2) | (m2) | (mg N) | (% w/w) | (kg/L) | (kg/L) | Rate (mL) | (L) | without N |
| 9.6 | 1076.02 | 0.0103 | 11.08 | 32% | 1.33 | 0.4256 | 0.0260 | 6 | 0.521 |
| 12 | 1345.02 | 0.0103 | 13.85 | 32% | 1.33 | 0.4256 | 0.0326 | 1 | 0.651 |

TABLE 4
Replacement Effect

| Treatment description | Replacement rate | Application rate equivalent |
|---|---|---|
| 80% GSP | — | — |
| 80% GSP + Biofertilizer 1.2 | 20% | 2 oz = 1 1b N |
| 80% GSP + Biofertilizer 1.2 D1 | 20% | 2 oz = 1 1b N |
| 100% GSP | — | — |

TABLE 5
Replacement and Boosting Effect

| Treatment description | Replacement rate | Application rate equivalent |
|---|---|---|
| 80% GSP | — | — |
| 80% GSP + Biofertilizer 1.2 | 20% | 2 oz = 1 1b N |
| 80% GSP + Biofertilizer 1.2 D1 | 20% | 2 oz = 1 1b N |
| 80% GSP + D1 | 20% | 2 oz = 1 1b N |
| 100% GSP | — | — |
| 100% GSP + Biofertilizer 1.2 | Boost | 2 oz = 1 1b N |
| 100% GSP + Biofertilizer 1.2 D1 | Boost | 2 oz = 1 1b N |
| 100% GSP + D1 | Boost | 2 oz = 1 1b N |

TABLE 6
Application Rate Comparison

| Treatment description | Replacement rate | Application rate equivalent |
|---|---|---|
| 80% GSP | — | — |
| 80% GSP + Biofertilizer 1.2 | 20% | 2 oz = 1 1b N |
| 80% GSP + Biofertilizer 1.2 D1 | 20% | 2 oz = 1 1b N |
| 80% GSP + Biofertilizer 1.2 | 20% | 8 oz = 1 1b N |
| 80% GSP + Biofertilizer 1.2 D1 | 20% | 8 oz = 1 1b N |
| 100% GSP | — | — |

TABLE 7
Diluent optimization - final cell density

| Treatment description | Replacement rate | Application rate equivalent |
|---|---|---|
| 80% GSP | — | — |
| 80% GSP + Biofertilizer 1.2 D1 | 20% | 2 oz = 1 1b N |
| Standard product @ 1e9 CFU/ml | | |
| 80% GSP + Biofertilizer 1.2 D1 @ | 20% | 2 oz = 1 1b N |
| 1e8 CFU/ml | | |
| 80% GSP + Biofertilizer 1.2 D1 @ | 20% | 2 oz = 1 1b N |
| 1e7 CFU/ml | | |
| 100% GSP | — | — |

Starting on day 5, weekly doses of 50 mL of 100% Hoagland's Solution without N were provided to supplement other nutrients.

### Diluent #1 Preparation

Diluent #1 was prepared as follows:

In a cloth bag, 20-110 g of dry alfalfa pellets were mixed with 1 L of 4°-95° C. water and steeped for 0.5-18 hrs. The resulting extract (4° C.-70° C.) was processed through 0.8/0.45 μm filter to remove suspended solids prior to sterile filtration though a 0.22 μm pore filter. Alternatively, steepage was autoclaved for 20 min at 121° C. 15 psi. The steepage was then stored at 4°-23° C. for use at the desired mixing ratio.

At time of harvest, the following was collected:
1) Aboveground imaging,
2) Aboveground Fresh and Dry Weight,
3) Belowground Dry Weight,
4) Root imaging (if necessary), and
5) WARD Nutritional data: Aboveground, Belowground & Soil.

### Results.

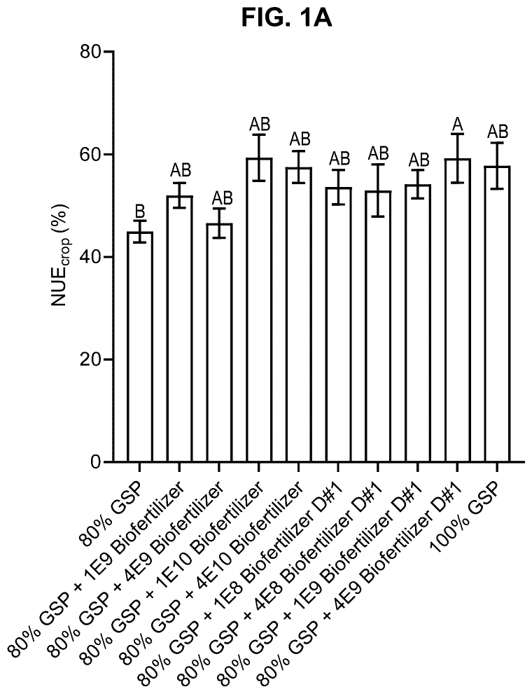

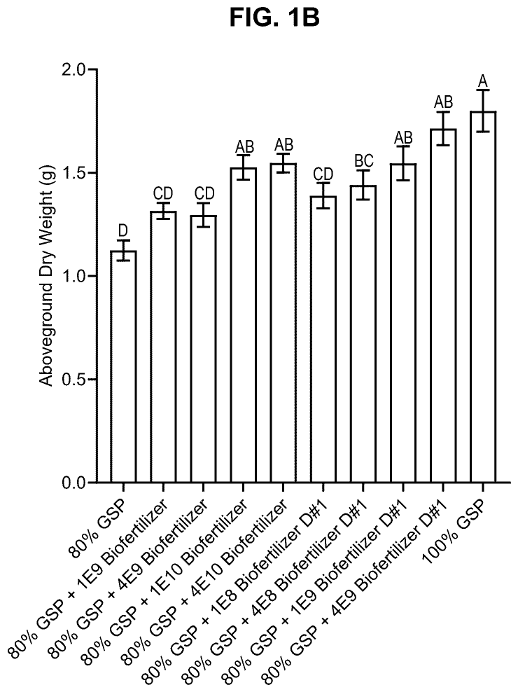

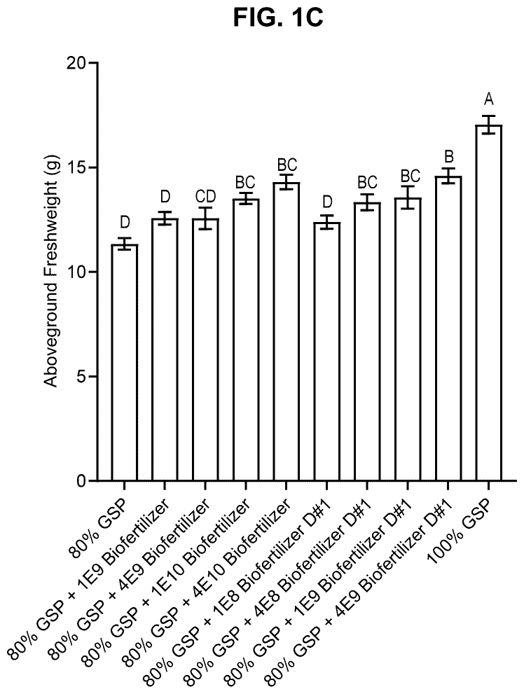

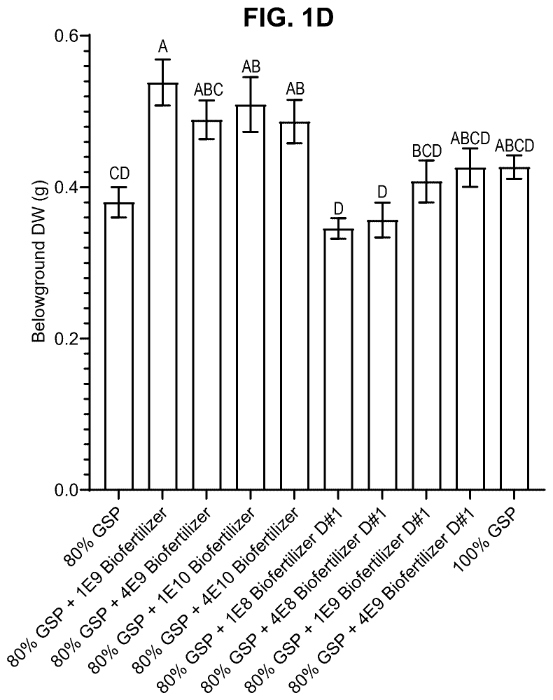

Nutrient data showed an increase in nitrogen utilization efficiency (NUE) in the group receiving the most concentrated biofertilizer in Diluent #1. See FIG. 1A. An increase in NUE suggested either an increased efficiency of nitrogen uptake, or (more likely in this case) a greater input of nitrogen into the system from nitrogen fixation.

Aboveground yields showed that the biofertilizer and the biofertilizer in Diluent #1 both show significant nitrogen replacement effects. Diluting with Diluent #1 lowers the threshold of CFUs at which a significant replacement effect was observed. Just looking at the aboveground fresh-weight, a significant replacement effect was observed when a total of 4.0E8 CFUs were delivered with the biofertilizer in Diluent #1, whereas the same level of replacement required 1.0E10 CFUs when the biofertilizer was undiluted. See FIGS. 1B-1C. Belowground dry weight showed a very different pattern, especially in the biofertilizer undiluted treatment group. However, belowground dry weight was not as strong of an indicator of nitrogen replacement effect. See FIG. 1D.

The purpose of this experiment was to observe a dose response in lettuce when applying the biofertilizer with an optimal application method (starter dose, weekly application of product). This experiment looked to both determine a minimal threshold at which the biofertilizer confers a benefit to lettuce, and test whether increasing the amount of the biofertilizer has a positive effect on plant growth.

Additional treatment groups were included to measure an optimal application rate with and without the 10× dilution into Diluent #1.

Total CFUs ranged from 1.0E9 to 4.0E10 for the undiluted biofertilizer groups and 1.0E8 to 4.0E9 for the Diluent #1 groups (10× dilution). Application volume was kept consistent throughout the study (total of 1 mL per plant per application).

Aboveground yield data showed a strong dose response for both biofertilizer and Biofertilizer/Diluent #1 treatment groups: as application increased, so too did yield. Treatments prepared in Diluent #1 showed a decreased threshold of CFUs at which significant nitrogen replacement occurred.

Treatments prepared in Diluent #1 had a lower threshold of CFUs at which significant nitrogen replacement occurred:
≥4.0E+8 CFUs in D#1 conferred significant N-replacement effect
≥1.0E+10 CFUs undiluted conferred significant N-replacement effect

The treatment group with the highest application rate in Diluent #1 had the highest nitrogen utilization efficiency, which suggested increased rates of nitrogen fixation and/or the ability to uptake nitrogen more efficiently.

These results suggest that 1.0E10 CFUs was an acceptable application rate for the biofertilizer undiluted. This experiment confirmed the optimal application rate of the biofertilizer undiluted: 1.0E+10 CFUs for a 4 week lettuce trial. This is equivalent to 20 oz/acre of product and was estimated to replace about 10 pounds N/acre.

Surprisingly, the experiment showed that 4.0E+8 CFUs in D#1 also conferred significant N-replacement effect. This allows for a reduction in the amount of CFUs needed to confer a yield response. For example, comparing 1) 1e10 CFU vs. 2) 4e8 CFUs in D#1 represents an unexpected 25 fold reduction in CFUs needed to achieve a yield response.

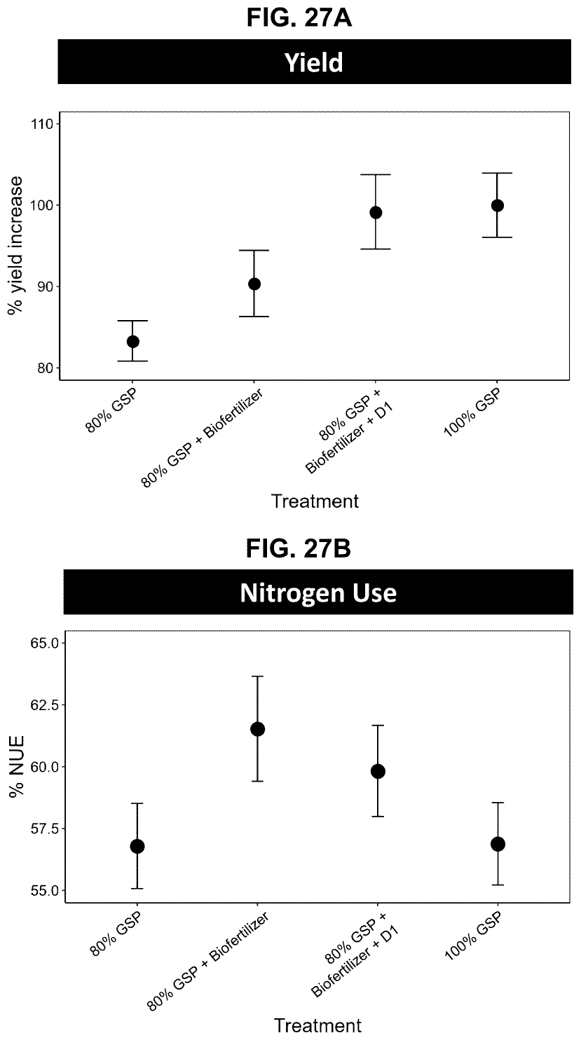

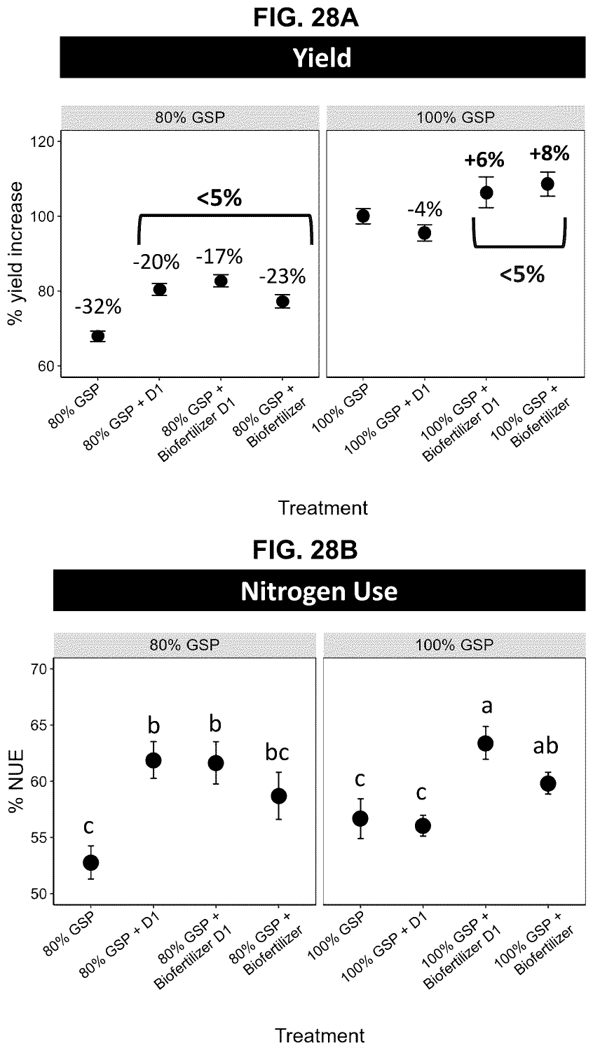

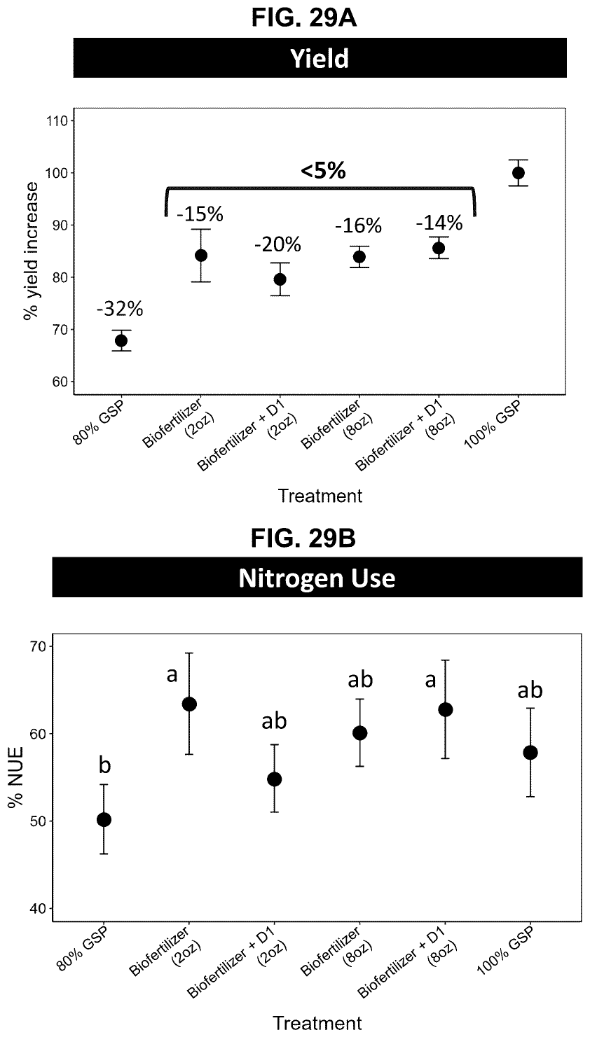

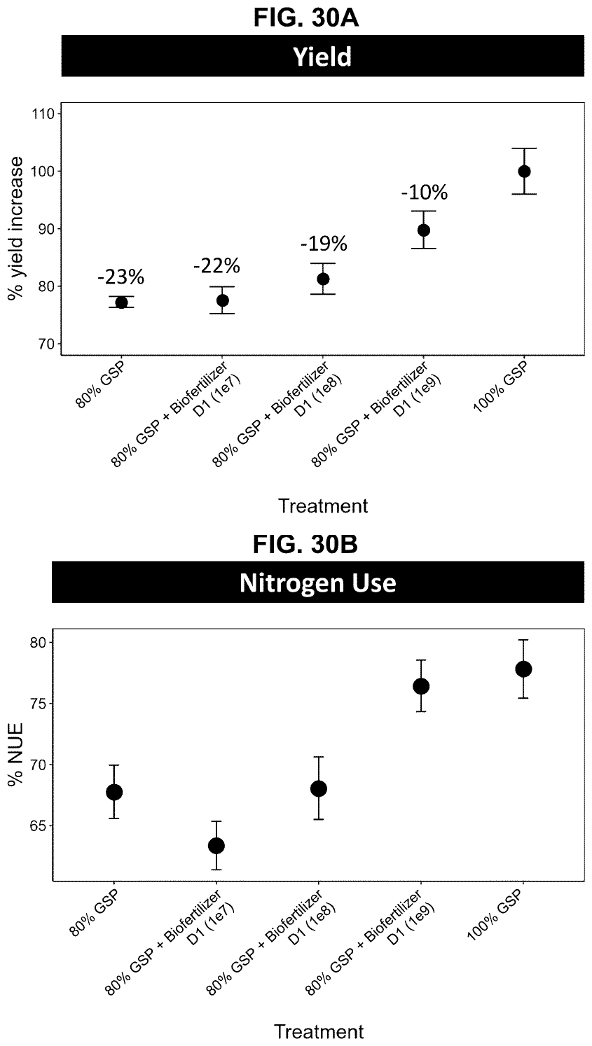

Additionally, Biofertilizer and Biofertilizer+D1 diluent showed higher % yield increase relative to 80% GSP, in addition to increased Nitrogen Use Efficiency (NUE) relative to both 80% GSP and 100% GSP. See FIGS. 27A-27B. Similar results were seen when 100% GSP was boosted with Biofertilizer or Biofertilizer+D1, while D1 alone did not show higher yield or NUE. See FIGS. 28A-28B. Replacement with 8 ounces of Biofertilizer+D1 saw minor improvements in yield and NUE relative to 2 ounces of Biofertilizer+D1, while no improvement was seen in 2 ounces of Biofertilizer alone compared to 8 ounces of Biofertilizer alone. See FIGS. 29A-29B. Finally, Biofertilizer+D1 applied at 1E9 CFUs saw the most improved yield and NUE. See FIGS. 30A-30B.

### Example 2—Diluent 1 Dilution Trial

The objective of this assay was to test the performance of the biofertilizer diluted in alfalfa tea. There were four treatments and the effects of adding the biofertilizer by volume as well as by CFU were compared. These four treatments of the biofertilizer and alfalfa tea were given to plants on top of a low nitrogen baseline of 80% GSP, and the plant response to these four treatments were compared to a low and high untreated nitrogen baseline.

### Experimental Design

72 plants were used, with 6 treatments for 6 plants in duplicate as described in Table 8. The treatments analyzed were:
1) Low N UTC: 80% GSP,
2) AK1: 80% GSP+1:10 Biofertilizer/D1 by volume,
3) AK2: 80% GSP+1:10 Biofertilizer/D1 by CFU,
4) K1: 80% GSP+Biofertilizer at 1e+9,
5) K2: 80% GSP+Biofertilizer at 1e+10, and
6) High N UTC: 100% GSP.

TABLE 8
Treatment protocol

| | | | | | Week 1 | | | Week 2 | | |
|---|---|---|---|---|---|---|---|---|---|---|
| | | | | | Day 0 | | Day 1 | Day 7 | | Day 8 |
| Treatment | Treatment | Total N | | | | volume | lb | | volume | lb |
| ID | description | Type | Ib N/ac | % GSP | CFU | (ml) | N/ac | CFU | (ml) | N/ac |
| Low | 80% GSP | UAN-32 | 38.4 | 80 | | | 9.6 | | | 9.6 |
| N UTC | | | | | | | | | | |
| AK1 | 80% GSP + 1:10 | UAN-32 | 38.4 | 80 | 4.00E+08 | 0.47 | 9.6 | 2.00E+08 | 0.23 | 9.6 |
| | Biofertilizer | | | | | | | | | |
| | in D#1; | | | | | | | | | |
| | added by volume | | | | | | | | | |
| AK2 | 80% GSP + 1:10 | UAN-32 | 38.4 | 80 | 4.00E+09 | 4.65 | 9.6 | 2.00E+09 | 2.33 | 9.6 |
| | Biofertilizer | | | | | | | | | |
| | in D#1; | | | | | | | | | |
| | added by CFU | | | | | | | | | |
| K1 | 80% GSP + | UAN-32 | 38.4 | 80 | 4.00E+08 | 0.05 | 9.6 | 2.00E+08 | 0.023 | 9.6 |
| | Biofertilizer | | | | | | | | | |
| | at D#1 | | | | | | | | | |
| | CFU | | | | | | | | | |
| K2 | 80% GSP + | UAN-32 | 38.4 | 80 | 4.00E+09 | 0.47 | 9.6 | 2.00E+09 | 0.23 | 9.6 |
| | Biofertilizer | | | | | | | | | |
| | at 1e10 | | | | | | | | | |
| High | 100% GSP | UAN-32 | 48 | 100 | | | 12 | | | 12 |
| N UTC | | | | | | | | | | |

| | Week 3 | | | Week 4 | | |
|---|---|---|---|---|---|---|
| | Day 14 | | Day 15 | Day 21 | | Day 22 |
| Treatment | | volume | lb | | volume | lb |
| ID | CFU | (ml) | N/ac | CFU | (ml) | N/ac |
| Low | | | 9.6 | | | 9.6 |
| N UTC | | | | | | |
| AK1 | 2.00E+08 | 0.23 | 9.6 | 2.00E+08 | 0.23 | 9.6 |
| AK2 | 2.00E+09 | 2.33 | 9.6 | 2.00E+09 | 2.33 | 9.6 |
| K1 | 2.00E+08 | 0.023 | 9.6 | 2.00E+08 | 0.023 | 9.6 |
| K2 | 2.00E+09 | 0.23 | 9.6 | 2.00E+09 | 0.23 | 9.6 |
| High | | | 12 | | | 12 |
| N UTC | | | | | | |

Plants dosed 50 mL of fertilizer blend: Hoagland's without nitrogen plus varying UAN-32 amounts. 80% GSP=9.6 lb N/ac/application: 0.0260 mL UAN32/pot/application. 100% GSP=12 lb N/ac/application: 0.0326 mL UAN32/pot/application. For low nitrogen, dosage was calculated for 64 plants (60+4 insurance). For high nitrogen, dosage was calculated for 13 plants (12+1 insurance). Each plant received 50 mL of Hoagland without nitrogen+UAN-32.

Fertilization was done weekly starting on days 1, 8, 15, 22 (after transplant).

Diluent 1 was prepared as follows:

In a cloth bag, 20-110 g of dry alfalfa pellets were mixed with 1 L of 4°-95° C. water and steeped for 0.5-18 hrs. The resulting extract (4° C.-70° C.) was processed through 0.8/0.45 μm filter to remove suspended solids prior to sterile filtration though a 0.22 μm pore filter. Alternatively, steepage was autoclaved for 20 min at 121° C. 15 psi. The steepage was then stored at 4°-23° C. for use at the desired mixing ratio.

Biofertilizer+D1 treatment (K1) was prepared by:
1) Pipet 0.05 mL HDB+4.95 mL water to each seedling for day 0, and
2) Pipet 0.023 mL HDB+4.977 mL water to each seedling for day 7, 14, 21.

Biofertilizer (K2) was prepared by:
1) Pipette 0.47 mL HDB+4.53 mL water to each seedling for day 0, and
2) Pipet 0.23 mL HDB+4.77 mL water to each seedling for day 7, 14, 21.

1:10 Biofertilizer/D1 treatment (AK1) was prepared by:
1) Pipette 0.47 mL Biofertilizer/Alfalfa tea Mixed solution+4.53 mL water to each seedling for day 0, and
2) Pipet 0.23 mL Biofertilizer/Alfalfa tea Mixed solution+4.77 mL water to each seedling for day 7, 14, 21.

1:10 Biofertilizer/D1 treatment (AK2) was prepared by:
1) Pipette 4.65 mL Biofertilizer/D1 Mixed solution+0.35 mL water to each seedling, and
2) Pipet 2.33 mL Biofertilizer/D1 Mixed solution+2.67 mL water to each seedling for day 7, 14, 21.

Treatments were done weekly starting on day 0, 7, 14, 21 (day of transplant).

### Results

The purpose of this assay was to test the effects of diluting the biofertilizer in Diluent 1 1:10 and adding this mixture, in treatments that accounted for either volume or CFU, to plants. These D1/biofertilizer treatments were compared to just biofertilizer treatments, and were given to plants on top of a low nitrogen baseline (80% GSP). There were also low and high controls of untreated 80% and 100% GSP plants.

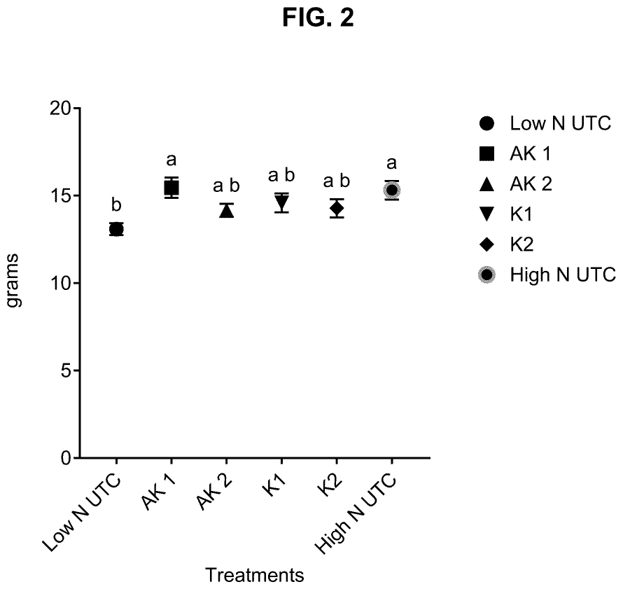

The results of this assay show that the only treatment that was significantly different from the low nitrogen baseline was AK1. AK1 was the only treatment that significantly increased aboveground biomass compared to the low nitrogen control, and was insignificantly different from the high nitrogen control. See FIG. 2.

There was no significant difference between the other treatments and the high nitrogen control, except between the low and the high nitrogen control.

In the case of AK1, at 80% GSP, the addition of Biofertilizer diluted in Diluent 1 for a total of 1E+09 CFUs was able to meet the nitrogen needs of the plants such that they were not significantly different from the high nitrogen control. Compared to the treatments AK2, where the same product was added based on CFU, we received a better result when we added biofertilizer diluted in Diluent 1 at 10 times lower the CFU.

The results of this assay indicate that the treatment that used Biofertilizer diluted in Diluent 1 for a total of 1E+09 CFUs delivered to the plant performed the best.

The tentative conclusion is that 1E+09 CFUs of Biofertilizer diluted in Diluent 1 is a treatment we should be targeting for further investigation.

### Example 3—Plant Response to Diluent Treatments

The purpose of this assay was to test the plant response of Diluent #1, the biofertilizer, and the biofertilizer diluted in Diluent #1 in lettuce at a low and high nitrogen baseline. The low nitrogen baseline was 80% GSP, and the high nitrogen baseline was 100% GSP. The treatment of the biofertilizer diluted ten times in Diluent #1 achieved a final concentration of 1E+09 CFU/mL.

The objective of this assay was to compare the effects of Diluent #1, the biofertilizer, and the biofertilizer diluted ten times in Diluent #1 in lettuce when grown with a low and high nitrogen baseline.

It was hypothesized that the plant response would be greater in the treatments that had the biofertilizer and Diluent #1 than treatments with Diluent #1 alone.

### Experimental Design

96 plants were used, with 8 treatments for 6 plants in duplicate, as shown in Table 9.

TABLE 9
Experimental Design

| | Treatment | Alfalfa | Treatment | | lb | |
|---|---|---|---|---|---|---|
| | ID | tea | description | Type | N/ac | % GSP |
| 1 | Low N UTC | | 80% GSP | UAN-32 | 38.4 | 80 |
| 2 | A1 | 4C | Diluent #1, added by volume | UAN-32 | 38.4 | 80 |
| 3 | AK1 | 4C | 80% GSP + 1:10 Biofertilizer in | UAN-32 | 38.4 | 80 |
| | | | Diluent #1; added by volume | | | |
| 4 | K1 | | 80% GSP + Biofertilizer at 1e10 | UAN-32 | 38.4 | 80 |
| 5 | High N UTC | | 100% GSP | UAN-32 | 48 | 100 |
| 6 | A2 | 4C | Diluent #1 tea; added by volume | UAN-32 | 48 | 100 |
| 7 | AK2 | 4C | 100% GSP + 1:10 Biofertilizer in | UAN-32 | 48 | 100 |
| | | | Diluent #1; added by volume | | | |
| 8 | K2 | | 100% GSP + Biofertilizer at 1e10 | UAN-32 | 48 | 100 |

4° C. Diluent #1 was prepared as follows:

In a cloth bag, 20-110 g of dry alfalfa pellets were mixed with 1 L of 4°-95° C. water and steeped for 0.5-18 hrs. The resulting extract (4° C.-70° C.) was processed through 0.8/0.45 μm filter to remove suspended solids prior to sterile filtration though a 0.22 μm pore filter. Alternatively, steepage was autoclaved for 20 min at 121° C. 15 psi. The steepage was then stored at 4°-23° C. for use at the desired mixing ratio.

Treatments were prepared as described in Table 10.

TABLE 10
Treatment protocol

| | | Volume of the | Volume of | | |
|---|---|---|---|---|---|
| | 24 Plants Total | biofertilizer | Diluent #1 | Volume of | Final Volumes |
| | (making enough | Needed per | Needed per | Water Needed | Needed per |
| Treatment | for 30) | Treatment | Treatment | Per Treatment | Feeding |
| K1, K2 (10x | 30 | 0.1 mL | | 0.9 mL | 3 mL |
| dilution of | | | | | biofertilizer, 27 |
| biofertilizer into | | | | | mL water |
| water) | | | | | |
| AK1, AK2 (10x | 30 | 0.01 mL | 0.09 mL | 0.9 mL | 0.3 mL |
| dilution of the | | | | | biofertilizer, 2.7 |
| biofertilizer into | | | | | mL Diluent #1, |
| Diluent #1, then | | | | | 27 mL water |
| 10x dilution of | | | | | |
| that into water) | | | | | |
| A1, A2 (10x | 30 | | 0.1 mL | 0.9 mL | 3 mL Diluent #1, |
| dilution of | | | | | 27 mL water |
| Diluent #1 into | | | | | |
| water) | | | | | |

N Fertilization was done weekly starting on day 0, 7, 14, 21 (day of transplant). Biofertilizer treatment and Diluent #1 was done weekly starting on day 1, 8, 15, 22 (day of transplant). N-Free Fertilization was done weekly starting on day 3, 10, 17, 24 (day of transplant). See Table 11.

TABLE 11
Treatment Protocol

| | Week 1 | | | Week 2 | | | Week 3 | | | Week 4 | | |
|---|---|---|---|---|---|---|---|---|---|---|---|---|
| Treatment | | Vol. | lb | | Vol. | lb | | Vol. | lb | | Vol. | lb |
| ID | CFU | (ml) | N/ac | CFU | (ml) | N/ac | CFU | (ml) | N/ac | CFU | (ml) | N/ac |
| Low N UTC | | | 9.6 | | | 9.6 | | | 9.6 | | | 9.6 |
| A1 | | | 9.6 | | | 9.6 | | | 9.6 | | | 9.6 |
| AK1 | 4.00E+08 | 0.47 | 9.6 | 2.00E+08 | 0.23 | 9.6 | 2.00E+08 | 0.23 | 9.6 | 2.00E+08 | 0.23 | 9.6 |
| K1 | 4.00E+09 | 0.47 | 9.6 | 2.00E+09 | 0.23 | 9.6 | 2.00E+09 | 0.23 | 9.6 | 2.00E+09 | 0.23 | 9.6 |
| High N UTC | | | 12 | | | 12 | | | 12 | | | 12 |
| A2 | | | 12 | | | 12 | | | 12 | | | 12 |
| AK2 | 4.00E+08 | 0.47 | 12 | 2.00E+08 | 0.23 | 12 | 2.00E+08 | 0.23 | 12 | 2.00E+08 | 0.23 | 12 |
| K2 | 4.00E+09 | 0.47 | 12 | 2.00E+09 | 0.23 | 12 | 2.00E+09 | 0.23 | 12 | 2.00E+09 | 0.23 | 12 |

### Results

The purpose of this experiment was to test the effect of 4° C. Diluent #1, the biofertilizer, and the biofertilizer diluted ten times in Diluent #1 in lettuce at 80% and 100% GSP. In the low nitrogen baseline plants, all treatments were significantly different from the UTC. Though there was no statistically significant difference between the treatments, the AK1 treatment with the biofertilizer diluted in Diluent #1 had the greatest fresh aboveground biomass. In the high nitrogen baseline plants, there was no statistical significance between the UTC and the three treatments. There was slight significance between the A2 and K2 treatments, and the AK2 and K2 treatments were both trending up from the UTC.

None of the treatments in the low nitrogen group were able to meet the high nitrogen group, but all were trending higher than the UTC. No significance between the treatments in the high nitrogen group, except between the A2 and K2 groups. None of the treatments with a low nitrogen baseline were able to match the high nitrogen UTC.

The results of this assay indicate that there was a positive plant response when treated with the biofertilizer diluted in Diluent #1, especially at the low nitrogen baseline. However, the results of other assays indicate that the effects could be greater when Diluent #1 steeped at a hotter temperature was used. Based on these results, Diluent #1 appeared to be a beneficial component for Biofertilizer to be diluted into.

All treatments with the low nitrogen baseline were significantly different from the low nitrogen UTC.

Greatest plant response in aboveground fresh weight was seen when the biofertilizer was diluted in Diluent #1 and applied on top of a low nitrogen baseline.

### Example 4—Expanded Dilution

The purpose of this experiment was to test further diluting the biofertilizer/D1 into more D1 or more water could yield any equivalent results as with the typically diluted (1.00E9 CFU/mL in D1) product. This experiment used biofertilizer and D1 prepared at a steeping temperature and concentration of 95° C. and 1×. This specific preparation of D1 has shown the most significant nitrogen replacement effects when mixed with the Biofertilizer product. This prepared mixture was diluted into water and D1 at 10× and 100× dilutions and applied to plants at the same application rate.

The objectives were as follows:

To demonstrate a robust nitrogen replacement effect at 80% GSP nitrogen levels with the addition of Biofertilizer/D1 at the traditional application rate (2 oz=1 lb N replaced) and cell density of final goods (1.00E9 CFU/mL).

To measure the relative nitrogen replacement when the product is diluted further into D1 or water (at 10× and 100× dilutions).

If a lower final cell density is used in the application of the biofertilizer products, then less robust nitrogen replacement effects was observed. However, if the product is diluted into D1, there was still some meaningful nitrogen replacement.

The goals were as follows:

Measure plant phenotypes to record plant health in response to varying fertilizer levels and microbial products.

Quantify nitrogen replacement in each treatment as % increase from the 80% GSP nitrogen control or as a % difference from the 100% GSP nitrogen control.

### Experimental Design

### Materials:

84-21-day-old lettuce seedlings
84-4″ pots
10 mL UAN-32
34 L N-Free Hoagland Solution
Coconut Coir+Field & Fairway blend
25 mL Biofertilizer D1
250 mL 1×95° C. D1

7 treatment groups with 12 plants per group were treated as described in Tables 12-17.

TABLE 12
Fertilizer dosage

| | Whole growing period | pre-heading | N difference | Application |
|---|---|---|---|---|
| | lb N/ac | lb N/ac | lb N/ac | ml Biofertilizer |
| 80% GSP | 96 | 36 | 12 | 1 |
| 100% GSP | 120 | 48 | 0 | 0 |

TABLE 13
Biofertilizer CFUs

| | Original | Diluted | |
|---|---|---|---|
| Biofertilizer product | CFUs | CFUs | Dilution |
| Biofertilizer D1 @ 1e+9 | 1.00E+09 | 1.00E+09 | D1 |
| Biofertilizer D1 in D1 @ 1e+8 | 1.00E+09 | 1.00E+08 | D1 |
| Biofertilizer D1 in D1 @ 1e+7 | 1.00E+09 | 1.00E+07 | D1 |
| Biofertilizer D1 in water @ 1e+8 | 1.00E+09 | 1.00E+08 | Sterile |
| | | | DI Water |
| Biofertilizer D1 in water @ 1e+7 | 1.00E+09 | 1.00E+07 | Sterile |
| | | | DI Water |

TABLE 14
Treatment description

| Treatment | Treatment | | lb | | | | |
|---|---|---|---|---|---|---|---|
| ID | description | Type | N/ac | % GSP | Dilution | CFUs | mL |
| 1 | 80% GSP_UTC | UAN-32 | 38.4 | 80 | | 0.00 | 0.00 |
| 2 | 80% GSP + Biofertilizer | UAN32 | 38.4 | 80 | None | 1.00E+09 | 1.00 |
| | D1 1e+9 | | | | | | |
| 3 | 80% GSP + Biofertilizer | UAN-32 | 38.4 | 80 | D1 | 1.00E+08 | 1.00 |
| | D1 1e+8 | | | | | | |
| 4 | 80% GSP + Biofertilizer | UAN32 | 38.4 | 80 | D1 | 1.00E+07 | 1.00 |
| | D1 1e+7 | | | | | | |
| 5 | 80% GSP + Biofertilizer | UAN-32 | 38.4 | 80 | Water | 1.00E+08 | 1.00 |
| | D1 1e+8 | | | | | | |
| 6 | 80% GSP + Biofertilizer | UAN32 | 38.4 | 80 | Water | 1.00E+07 | 1.00 |
| | D1 1e+7 | | | | | | |
| 7 | 100% GSP in CC | UAN-32 | 48 | 100 | | 0.00 | 0.00 |

TABLE 15
Treatment timing

| | Week 1 (Day 0) | | Week 2 (Day 7) | | Week 3 (Day 14) | | Week 4 (Day 21) | |
|---|---|---|---|---|---|---|---|---|
| Treatment | | Biofertilizer | | Biofertilizer | | Biofertilizer | | Biofertilizer |
| ID | CFU | (mL) | CFU | (mL) | CFU | (mL) | CFU | (mL) |
| 1 | 0.00E+00 | 0.0 | 0.00E+00 | 0.0 | 0.00E+00 | 0.0 | 0.00E+00 | 0.0 |
| 2 | 4.00E+08 | 0.4 | 2.00E+08 | 0.2 | 2.00E+08 | 0.2 | 2.00E+08 | 0.2 |
| 3 | 4.00E+07 | 0.4 | 2.00E+07 | 0.2 | 2.00E+07 | 0.2 | 2.00E+07 | 0.2 |
| 4 | 4.00E+06 | 0.4 | 2.00E+06 | 0.2 | 2.00E+06 | 0.2 | 2.00E+06 | 0.2 |
| 5 | 4.00E+07 | 0.4 | 2.00E+07 | 0.2 | 2.00E+07 | 0.2 | 2.00E+07 | 0.2 |
| 6 | 4.00E+06 | 0.4 | 2.00E+06 | 0.2 | 2.00E+06 | 0.2 | 2.00E+06 | 0.2 |
| 7 | 0.00E+00 | 0.0 | 0.00E+00 | 0.0 | 0.00E+00 | 0.0 | 0.00E+00 | 0.0 |

TABLE 16
Stock dilution

| Stock | Dilution | Total (mL) | Product (mL) | Water (mL) | D#1 (mL) |
|---|---|---|---|---|---|
| 80% GSP + Biofertilizer D1 1e+9 | None | 15 | 15 | | |
| 80% GSP + Biofertilizer D1 1e+8 | D1 | 15 | 1.5 | | 13.5 |
| 80% GSP + Biofertilizer D1 1e+7 | D1 | 15 | 0.15 | | 14.85 |
| 80% GSP + Biofertilizer D1 1e+8 | Water | 15 | 1.5 | 13.5 | |
| 80% GSP + Biofertilizer D1 1e+7 | Water | 15 | 0.15 | 14.85 | |

TABLE 17
UAN application

| | | | Weekly | | | |
|---|---|---|---|---|---|---|
| | | | (starting day 1) | | | |
| Treatment | Treatment | Total N | lb | | | lb |
| ID | description | Type | N/ac | % GSP | mL/pot | N/ac |
| 1 | 80% GSP_UTC | UAN-32 | 38.4 | 80 | 0.0264 | 9.6 |
| 2 | 80% GSP + Biofertilizer | UAN32 | 38.4 | 80 | 0.0264 | 9.6 |
| | D1 1e+9 | | | | | |
| 3 | 80% GSP + Biofertilizer | UAN-32 | 38.4 | 80 | 0.0264 | 9.6 |
| | D1 1e+8 | | | | | |
| 4 | 80% GSP + Biofertilizer | UAN32 | 38.4 | 80 | 0.0264 | 9.6 |
| | D1 1e+7 | | | | | |
| 5 | 80% GSP + Biofertilizer | UAN-32 | 38.4 | 80 | 0.0264 | 9.6 |
| | D1 1e+8 | | | | | |
| 6 | 80% GSP + Biofertilizer | UAN32 | 38.4 | 80 | 0.0264 | 9.6 |
| | D1 1e+7 | | | | | |
| 7 | 100% GSP in CC | UAN-32 | 48 | 100 | 0.0330 | 12 |

### Data Collection

### Physical Data:

1) Aboveground Fresh-Weight and Dry-Weight
2) Belowground Dry-Weight
3) Scan plants at time of Harvest by Traitfinder (data not shown)
4) Photos at time of Harvest (data not shown)

### Nutrient Data:

1) Total Nitrogen of leaves (data not shown)
2) NUE calculation

### Results

Given a batch of biofertilizer mixed with 95° C. 1×D1, this experiment looked to further dilute this stock in D1 and water (10× and 100× treatments) to determine if there was any significant nitrogen replacement effects with more dilute products, and if using water or more D1 had any effect. These treatment groups were all applied to treatments at an 80% GSP nitrogen baseline, and compared to untreated controls at 80% and 100% GSP nitrogen levels to measure nitrogen replacement.

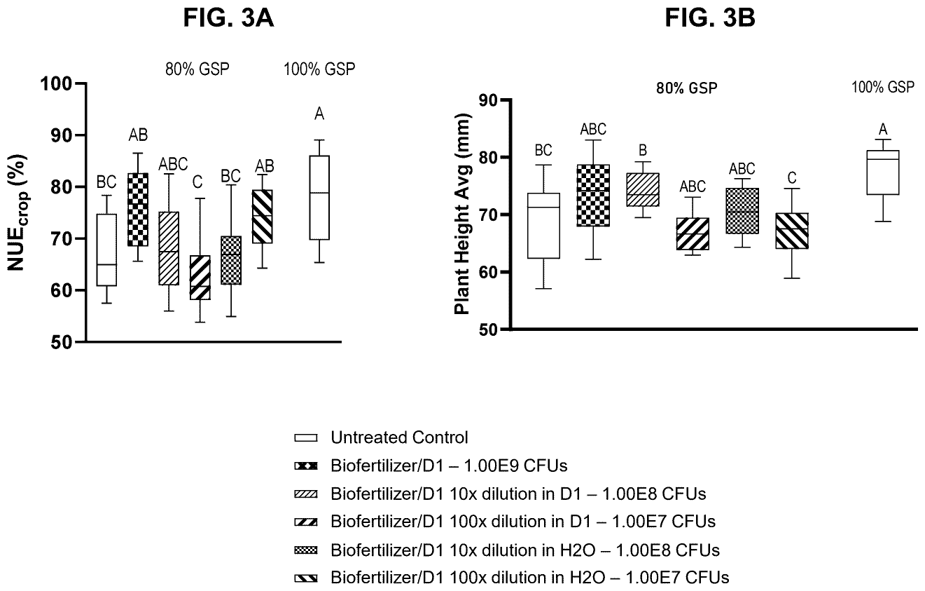

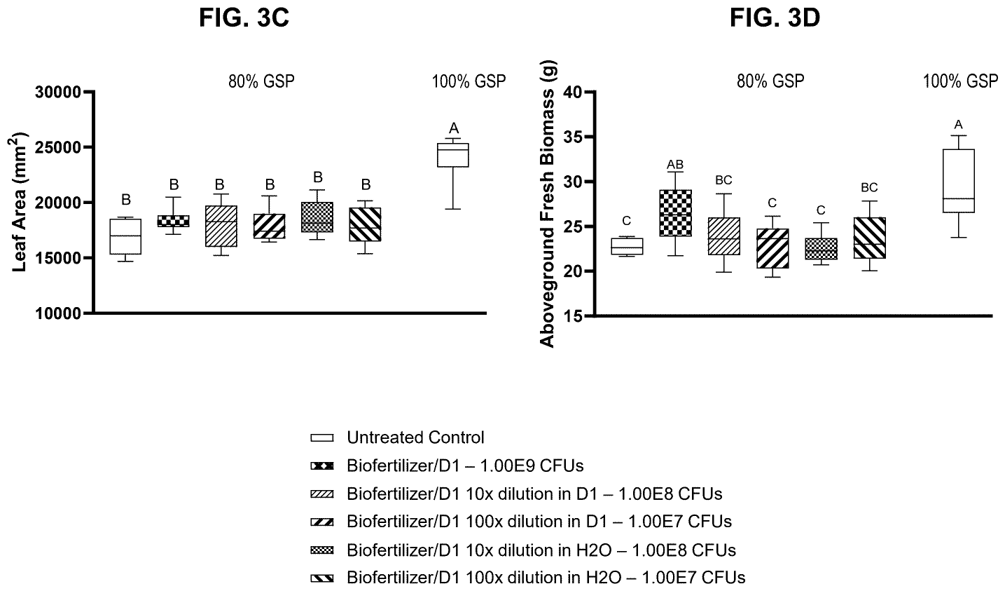

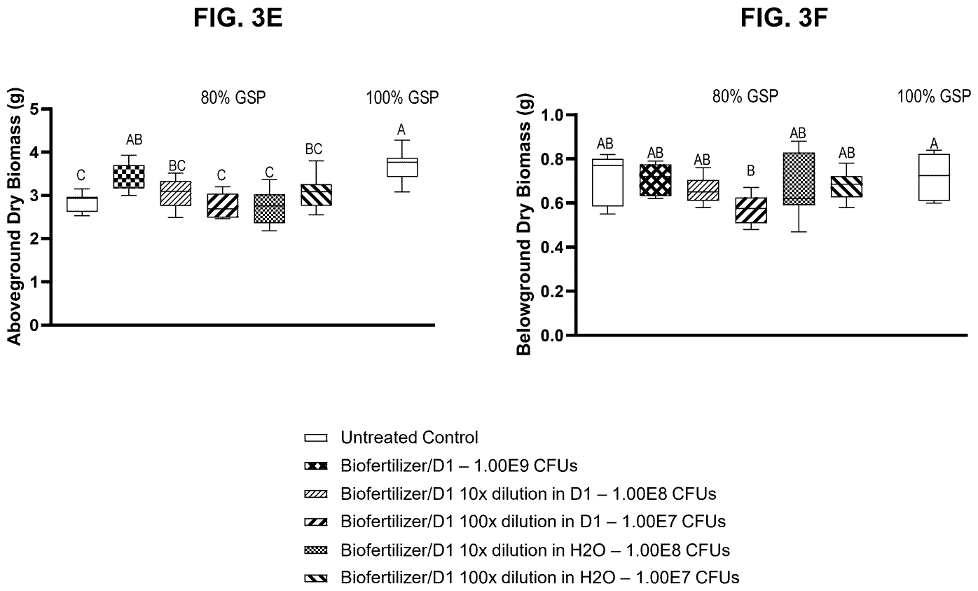

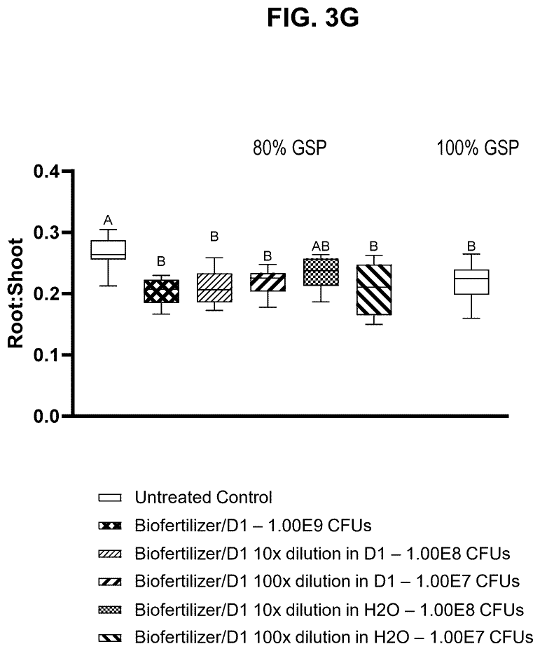

The only treatment that showed significant nitrogen replacement in terms of yield was the treatment that received the standard final goods product diluted to 1.00E9 CFUs in D1. Further dilution into water/D1 didn't provide any significant difference in aboveground yield relative to the control. See FIGS. 3A-3G.

Nutrient data reflected a higher Nitrogen Utilization Efficiency in the treatment group receiving the 1.00E9 CFUs in D1 product. This further supported that this treatment was the top performing of all the treatments included in this trial and indicates the potential for additional nitrogen being introduced into each pot via nitrogen fixation of Xanthobacter.

This suggests that this approximate final cell density was as low as possible, while still producing a significant nitrogen replacement effect. These results also suggested that D1 helped to facilitate the nitrogen-replacement effect of Xanthobacter since there was little benefit of adding a higher proportion of D1 relative to the total amount of Xanthobacter autotrophicus. If D1 itself had a large effect on plant yield, there would likely be a larger boost to plant yield from the treatments receiving biofertilizer diluted further into D1. Instead, the highest performing treatment is with biofertilizer and D1. The additive effects of these two components is demonstrated by the following results seen throughout this and other previous experiments:
1) biofertilizer at 1.00E9 CFUs/mL diluted into water showed less significant nitrogen replacement results than Biofertilizer diluted into D1 to the same final cell density; and
2) Further dilution of the product into D1, and performance of D1 alone showed no significant nitrogen replacement.

### Example 5—Additional Diluent Testing

In Examples 5-10, additional data relating to claims and examples of diluents used with microbial fertilizers to increase efficacy and/or shelf-life was analyzed. Initial methods are described in Table 18.

TABLE 18
Summary table of additional results including examples tested, data
generated, and recommended concentrations for each example.

| | | Data Generated | | | | | |
|---|---|---|---|---|---|---|---|
| | | Shelf- | | Tissue | Pot | | |
| Example | Description | life | LRA | Culture | Trial | Concentration | |
| Tomato extract | Freeze dried, | — | ✓ | — | ✓ | 0.218-1.15 | g/L |
| | blended tomato | | | | | | |
| Orange extract | Freeze dried, | — | ✓ | — | ✓ | 2.198-3.558 | g/L |
| | ground orange peel | | | | | | |
| Rice extract | Rice hull extract | ✓ | — | ✓ | — | 5.26 g/L-42.2 g/L | |
| Algae extract | Chlorella extract | — | ✓ | — | ✓ | 0.4-4.0 | g/L |
| Yeast extract | Yeast extract | — | ✓ | — | ✓ | 0.4-4.0 | g/L |
| | Brewer's yeast | — | ✓ | — | — | 0.4-4.0 | g/L |
| Insect/Crustacean | Chitosan | — | ✓ | — | ✓ | 50-500 | mg/L |
| Extract combo | Chlorella + Yeast extract | — | ✓ | — | ✓ | 0.4 g/L + 0.4 g/L | |
| Microbial combo | X. autotrophicus + | — | ✓ | — | — | 21 | g/L |
| | another bacteria + | | | | | | |
| | Alfalfa Extract | | | | | | |

### Preparation of Tested Materials

Tomato extract: Dried tomatoes were ground into a fine powder using a spice grinder. The resulting powder was added to water in accordance with prior analysis to match the composition of the in-house diluent D1, which results in a range of 0.218-1.15 g/L.

Orange peel extract: Dried orange peels were ground into a fine powder using a spice grinder. The resulting powder was added to water in accordance with prior analysis to match the composition of the in-house diluent, which results in a range of 2.198-3.558 g/L.

Chlorella extract: Chlorella powder was added to heated water and stirred until well dispersed. The suspension was then filtered to remove insoluble material. The resulting liquid extract was prepared at various concentrations for testing in the range of 0.4-4.0 g/L.

Yeast extract: Yeast extract powder was dissolved in heated water, stirring to ensure complete solubilization. The solution was filtered to remove any undissolved particulates and prepared at various concentrations for testing in the range of 0.4-4.0 g/L.

Brewer's yeast: Brewer's yeast was suspended in heated water and stirred thoroughly. The solution was filtered to remove any undissolved particulates and prepared at various concentrations for testing in the range of 0.4-4.0 g/L.

Chitosan: Chitin powder was suspended in heated water and stirred continuously to aid dispersion. The solution was filtered to remove any undissolved particulates and prepared at various concentrations for testing in the range of 50-500 mg/L

Extract combo: Liquid Chlorella extract and liquid yeast extract were prepared as described above at a concentration of 1.0 g/L, respectively. Chlorella and yeast extracts were combined in equal volumes. Combined extract was then diluted 5× and combined with X. autotrophicus cells for testing as described below.

Microbial combo: Microbial mixtures were made containing X. autotrophicus and one of the following: Pseudomonas fluorescens, Rhodopseudomonas palustris, Azospirillum lipoferum, Cupriavidus necator. Microbial mixtures were combined with alfalfa extract prepared at 21 g/L as a diluent and cell concentration was adjusted to 5e8 CFU/ml for X. autotrophicus and 5e8 CFU/ml for the other species.

Table 19 shows Ion composition of tomato extract and orange peel extract compared to D1. These values were used to calculate estimated mass per volume of tomato and orange peel extract to match D1.

TABLE 19
Ion composition of D1, tomato extract, orange peel extract.

| | | Tomato | Orange Peel |
|---|---|---|---|
| | D1 | Extract | Extract |
| | (21 g/L) | (21 g/L) | (21 g/L) |
| Nitrate | 25.5 | 1958.0 | 706.3 |
| Phosphate | 59.5 | 64.3 | 18.9 |
| Sodium | 46.1 | 35.0 | 139.9 |
| Potassium | 428.7 | 3587.4 | 783.2 |
| Magnesium | 53.1 | 174.8 | 146.9 |
| Calcium | 154.2 | 279.7 | 342.7 |

### Example 6—Tomato and Orange Peel Extracts

The purpose of this experiment was to test the use of Tomato and Orange peel extracts as examples of diluent for enhancing lateral root development by microbial fertilizers. Lateral root development was assessed in romaine lettuce seedlings (Lactuca sativa var. salivus, Johnny's Selected Seeds, Winslow, Maine, USA) as follows. 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach. Seeds were exposed to bleach solution for 10 minutes after which the bleach solution was aspirated, and the seeds were washed with 1 ml of sterile water six times. Seeds were then resuspended 1 ml sterile water and placed at 4° C. for 48 hours to stratify after which the water was aspirated, and seeds were stored dry at 4° C. for up to 2 weeks before sowing. Sterile lettuce seeds were placed near the top of the 50% Hoagland's agar plate along the “sowing line”. Using a pipette, 30 μl of either water (negative control) or tested product (according to Table 20 below), were plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at a ˜80° angle during growth to encourage root growth along the agar surface. Seedlings were grown for 7 days and then root development was assessed as lateral root density determined by dividing the lateral root number of total tap root length.

TABLE 20
Methods for Orange and Tomato Extract

| | | Diluent |
|---|---|---|
| Treatment # | Treatment Description | Tested |
| 1 | Negative control (Water Only) | — |
| 2 | Biofertilizer + D1 | Alfalfa (21 g/L) |
| 3 | Biofertilizer + Tomato Extract ME | Tomato (1.15 g/L) |
| 4 | Biofertilizer + Tomato Extract DE | Tomato (0.218 g/L) |
| 5 | Biofertilizer + Orange Peel | Orange Peel |
| | Extract ME | (3.558 g/L) |
| 6 | Biofertilizer + Orange Peel | Orange Peel |
| | Extract DE | (2.198 g/L) |

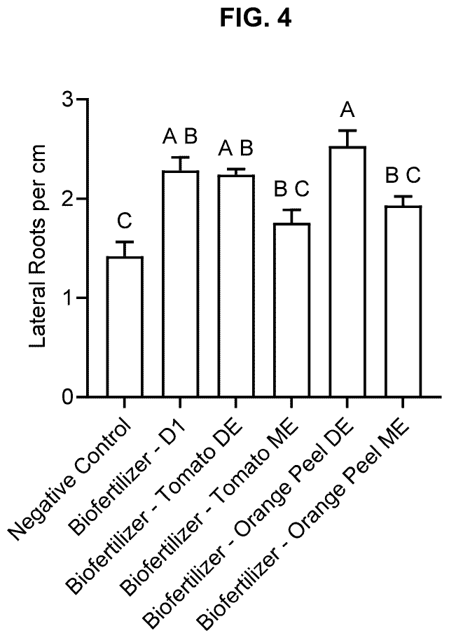

Application of Biofertilizer+D1 increased lateral root density of lettuce seedlings compared to the negative control (FIG. 4). This expected result validates the outcomes of this assay. Application of Biofertilizer prepared with tomato or orange peel extract increased lateral root density compared to the negative control regardless of diluent concentration. However, only tomato extract at 0.218 g/L and orange peel extract at 2.198 g/L significantly increased lateral root density compared to the negative control.

### Tomato and Orange Peel Extract Effect on Biofertilizer.

The purpose of this experiment was to investigate the efficacy of Biofertilizer products prepared using Tomato and Orange peel extract as diluents compared to standard Biofertilizer+D1 product on lettuce. Romaine lettuce seedlings (Lactuca sativa var. salivus, Johnny's Selected Seeds, Winslow, Maine, USA) are sown into trays containing sunshine mix #1. Once germinated, seedlings are watered as needed and provided weekly doses of 50% Hoagland's solution with nitrogen to support growth. Seedlings are grown for 21-days before transplanting for the experimental trial. Seedlings are transplanted into 4″ pots containing coconut coir potting media. Lettuce plants are grown for 28 days from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 80% grower standard practice (GSP) or 100% GSP according to the treatments listed in Table 21 below. Standard Biofertilizer+D1 or Biofertilizer made with different diluents was applied as described below. Plants are watered as needed during the growth period. After 28 days of growth, lettuce plants are harvested for aboveground fresh biomass, aboveground dry biomass, and leaf tissue nitrogen (N). Leaf tissue nitrogen (%) was used to determine total leaf nitrogen by multiplying percent nitrogen and aboveground dry biomass. Nitrogen use efficiency crop (NUEcrop) was calculated as total leaf nitrogen divided by nitrogen supplied as fertilizer.

TABLE 21
Tomato and Orange Peel extract treatment

| Treatment | | N equiv. | Biofertilizer | |
|---|---|---|---|---|
| # | Treatment Description | (lb/ac) | Diluent | Rate |
| 1 | 80% GSP | 36 | — | — |
| 2 | 80% GSP + Biofertilizer + D1 | 36 | Alfalfa (21 g/L) | 8 oz/ac |
| 3 | 80% GSP + Biofertilizer + Tomato | 36 | Tomato (1.15 g/L) | 8 oz/ac |
| | Extract ME | | | |
| 4 | 80% GSP + Biofertilizer + Tomato | 36 | Tomato (0.218 g/L) | 8 oz/ac |
| | Extract DE | | | |
| 5 | 80% GSP + Biofertilizer + Orange | 36 | Orange Peel (3.558 g/L) | 8 oz/ac |
| | Peel Extract ME | | | |
| 6 | 80% GSP + Biofertilizer + Orange | 36 | Orange Peel (2.198 g/L) | 8 oz/ac |
| | Peel Extract DE | | | |
| 7 | 100% GSP | 48 | — | — |

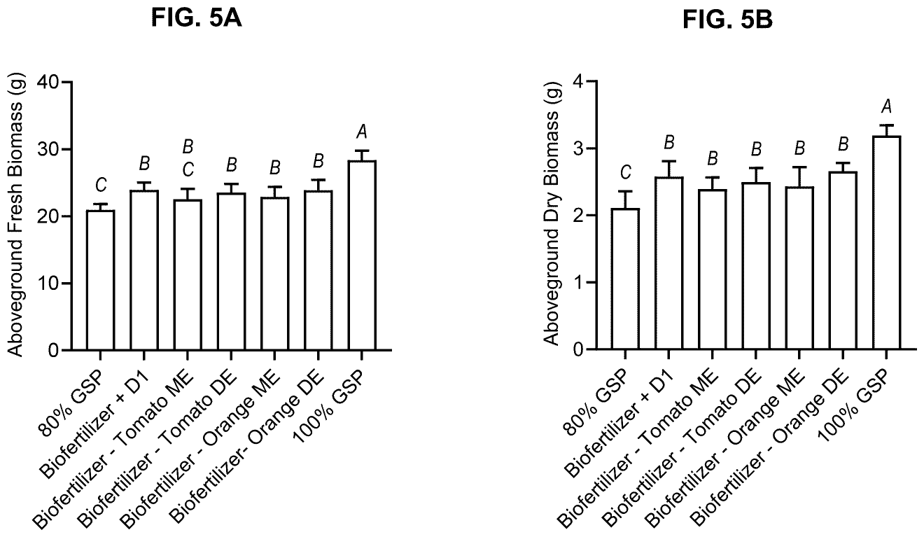

Application of Standard Biofertilizer+D1 significantly increased fresh and dry biomass of lettuce compared to 80% GSP control. Application of Biofertilizer prepared with tomato or orange peel extract also significantly increased aboveground fresh and dry biomass of lettuce compared to 80% GSP control (FIGS. 5A-5B). Of these treatments, the use of tomato extract at 0.218 g/L and orange peel extract at 2.198 g/L resulted in slightly greater increases in biomass compared to the other concentrations of those extracts.

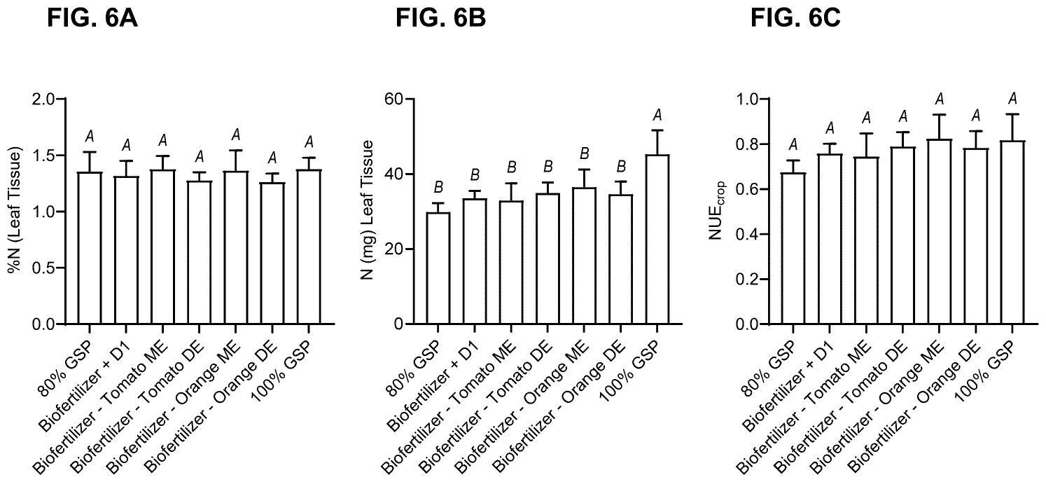

Furthermore, leaf tissue nitrogen content, total leaf nitrogen, and nitrogen use efficiency of lettuce was comparable to control (FIGS. 6A-6C).

### Example 7—Yeast Extract and/or Chlorella Extracts as a Diluent

The purpose of this experiment was to test the use of Yeast, Brewer's yeast, Chlorella, and Yeast+Chlorella extracts as examples of diluent for enhancing lateral root development by microbial fertilizers. Lateral root development was assessed in romaine lettuce seedlings (Lactuca sativa var. salivus, Johnny's Selected Seeds, Winslow, Maine, USA) as follows. 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach. Seeds were exposed to bleach solution for 10 minutes after which the bleach solution was aspirated, and the seeds were washed with 1 ml of sterile water six times. Seeds were then resuspended 1 ml sterile water and placed at 4° C. for 48 hours to stratify after which the water was aspirated, and seeds were stored dry at 4° C. for up to 2 weeks before sowing. Sterile lettuce seeds were placed near the top of the 50% Hoagland's agar plate along the “sowing line”. Using a pipette, 30 μl of either water (negative control) or tested product (according to Table 22 below), were plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at a ˜80° angle during growth to encourage root growth along the agar surface. Seedlings were grown for 7 days and then root development was assessed as lateral root density determined by dividing the lateral root number of total tap root length.

TABLE 22
Methods for yeast extract and chlorella extract analysis

| Treatment # | Treatment Description | Diluent Tested |
|---|---|---|
| 1 | Negative control (Water Only) | — |
| 2 | Biofertilizer + D1 | Alfalfa (21 g/L) |
| 3 | Biofertilizer + Brewer's | Brewer's Yeast (0.4 g/L) |
| | Yeast Extract | |
| 4 | Biofertilizer + Yeast Extract | Yeast (0.4 g/L) |
| 5 | Biofertilizer + Chlorella Extract | Chlorella (0.4 g/L) |
| 6 | Biofertilizer t + | Yeast (0.4 g/L) + |
| | Yeast:Chlorella Extract | Chlorella (0.4 g/L) |

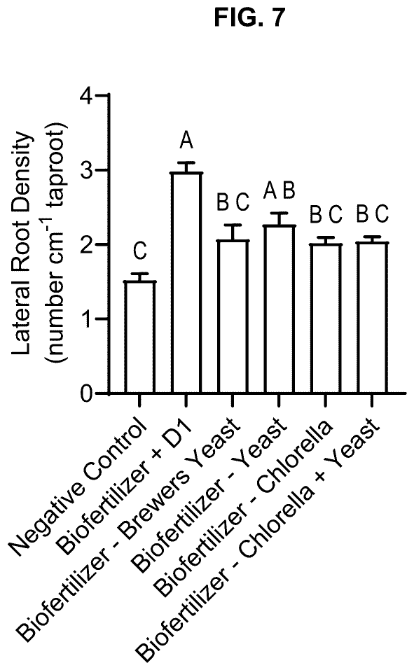

Application of Standard Biofertilizer+D1 increased lateral root density of lettuce seedlings compared to the negative control (FIG. 7). This expected result validates the outcomes of this assay. Application of Biofertilizer prepared with alternative diluent produced results that were in between the controls, with the use of Yeast Extract (0.4 g/L) as a diluent resulting in the most lateral roots per cm in compared to other candidate diluents.

Lateral Root Development after Treatment with Diluent Derived from Yeast, Brewer's Yeast, Chlorella, and/or Yeast+Chlorella Extracts.

The purpose of this experiment was to test the use of Yeast, Brewer's yeast, Chlorella, and Yeast+Chlorella extracts as examples of diluent for enhancing lateral root development by microbial fertilizers. Lateral root development was assessed in romaine lettuce seedlings (Lactuca sativa var. salivus, Johnny's Selected Seeds, Winslow, Maine, USA) as follows. 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach. Seeds were exposed to bleach solution for 10 minutes after which the bleach solution was aspirated, and the seeds were washed with 1 ml of sterile water six times. Seeds were then resuspended 1 ml sterile water and placed at 4° C. for 48 hours to stratify after which the water was aspirated, and seeds were stored dry at 4° C. for up to 2 weeks before sowing. Sterile lettuce seeds were placed near the top of the 50% Hoagland's agar plate along the “sowing line”. Using a pipette, 30 μl of either water (negative control) or tested product (according to Table 23 below), were plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at a ˜80° angle during growth to encourage root growth along the agar surface. Seedlings were grown for 7 days and then root development was assessed as lateral root density determined by dividing the lateral root number of total tap root length.

TABLE 23
Lateral root development treatments

| Treatment # | Treatment Description | Diluent Tested |
|---|---|---|
| 1 | Negative control (Water Only) | — |
| 2 | Standard Biofertilizer + D1 | Alfalfa (21 g/L) |
| 3 | Biofertilizer + YEL | Yeast Extract (0.2 g/L) |
| 4 | Biofertilizer + YEM | Yeast Extract (0.4 g/L) |
| 5 | Biofertilizer + YEH | Yeast Extract (0.8 g/L) |
| 6 | Biofertilizer + Chitosan | Chitosan (40 mg/L) |

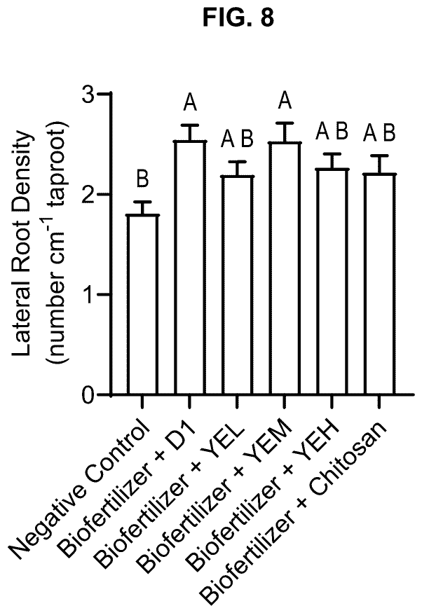

Application of Standard Biofertilizer+D1 increased lateral root density of lettuce seedlings compared to the negative control (FIG. 8). This expected result validates the outcomes of this assay. The application of Biofertilizer prepared with 0.4 g/L yeast extract resulted in similar lateral root density as standard Biofertilizer. Only standard Biofertilizer+D1 and Biofertilizer prepared with 0.4 g/L of yeast extract statistically increased lateral root density compared to the negative control. All other diluent candidates resulted in a non-significant increase in lateral root density compared to the negative control.

### Diluent Effect on Seedlings.

The purpose of this experiment was to investigate the efficacy of Biofertilizer products prepared using Yeast extract, Chlorella extract, Yeast extract+Chlorella extract, or Chitosan as diluents compared to standard Biofertilizer+D1 product on lettuce. Romaine lettuce seedlings (Lactuca sativa var. salivus, Johnny's Selected Seeds, Winslow, Maine, USA) are sown into trays containing sunshine mix #1. Once germinated, seedlings are watered as needed and provided weekly doses of 50% Hoagland's solution with nitrogen to support growth. Seedlings are grown for 21-days before transplanting for the experimental trial. Seedlings are transplanted into 4″ pots containing coconut coir potting media. Lettuce plants are grown for 28 days from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 80% grower standard practice (GSP) or 100% GSP according to the treatments listed in Table 24 below. Standard Biofertilizer+D1 or Biofertilizer made with different diluents was applied as described below. Plants are watered as needed during the growth period. After 28 days of growth, lettuce plants are harvested for aboveground fresh biomass, aboveground dry biomass, and leaf tissue nitrogen (N). Leaf tissue nitrogen (%) was used to determine total leaf nitrogen by multiplying percent nitrogen and aboveground dry biomass. Nitrogen utilization efficiency crop (NUE_{crop}) was calculated as total leaf nitrogen divided by nitrogen supplied as fertilizer.

TABLE 24
Seedling treatments

| Treatment | | N equiv. | Biofertilizer | |
|---|---|---|---|---|
| # | Treatment Description | (lb/ac) | Diluent | Rate |
| 1 | 80% GSP | 36 | — | — |
| 2 | 80% GSP + Biofertilizer + D1 | 36 | Alfalfa (21 g/L) | 8 oz/ac |
| 3 | 80% GSP + Biofertilizer with cells only | 36 | — | 8 oz/ac |
| 4 | 80% GSP + Biofertilizer + Yeast | 36 | Yeast (0.4 g/L) | 8 oz/ac |
| 5 | 80% GSP + Biofertilizer + Chlorella | 36 | Chlorella (0.4 g/L) | 8 oz/ac |
| 6 | 80% GSP + Biofertilizer + Chlorella + | 36 | Chlorella (0.2 g/L) + | 8 oz/ac |
| | Yeast | | Yeast (0.2 g/L) | |
| 7 | 80% GSP + Biofertilizer + Chitosan | 36 | Chitosan (40 mg/L) | 8 oz/ac |
| 8 | 100% GSP | 48 | — | — |

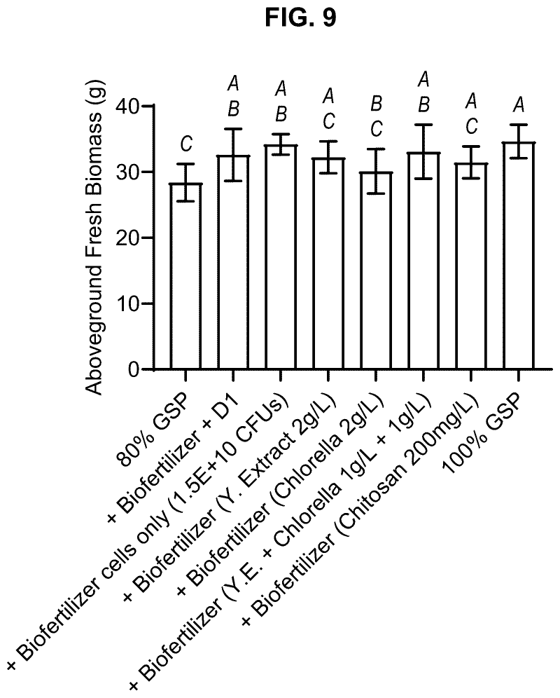

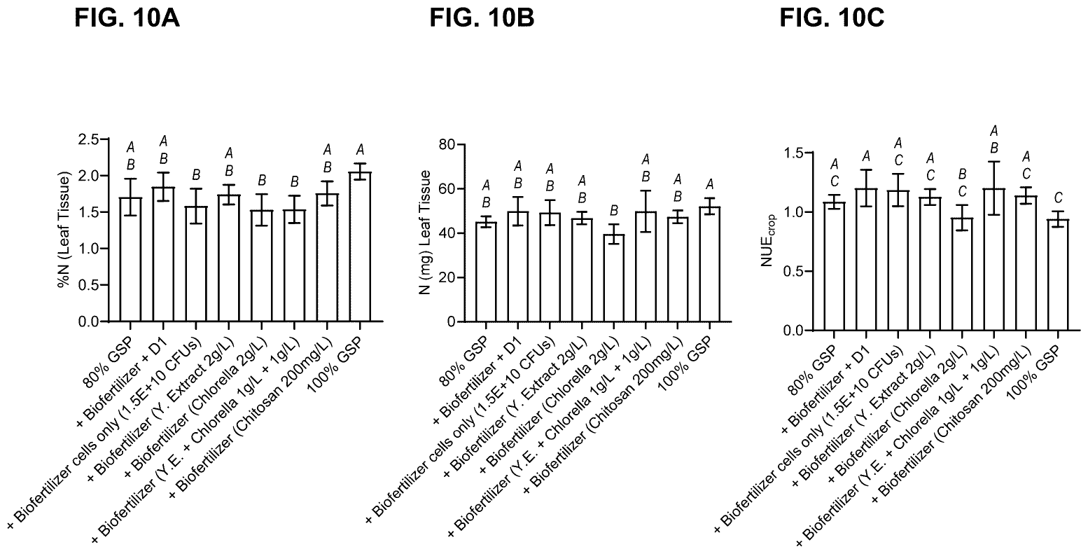

Application of standard Biofertilizer+D1 and Biofertilizer with cells only at 10× fold higher cell concentration significantly increased lettuce aboveground biomass over the 80% GSP control and was statistically similar to the 100% GSP control (FIG. 9). Biofertilizer prepared with Yeast extract, Chlorella+Yeast Extract, and Chitosan increased aboveground biomass compared to the 80% GSP control, but this increase was not statistically significant. FIGS. 10A-10C show Leaf tissue nitrogen content (FIG. 10A), total leaf nitrogen (FIG. 10B), and nitrogen use efficiency crop (FIG. 10C) data of lettuce grown in pots.

### Example 8—Rice Hull as a Diluent

### Brewer's Diluent Preparation.

Purpose: Determine optimal mixing ratios of rice hulls (RH or rice husk) for D2 preparation. Different concentrations (1×, 2×, 5×) were based on the standard diluent (alfalfa extract, D1) weight by volume ratio of 21.1 g/L. D2 formulation of 21.1 g/L is also labeled as a 1× concentration. Formulations were prepared with DI water.

### Physical Properties.

Design: Four 1 L batches of RH in distilled water were prepared to test physical properties of RH during extraction. Formulations were made in an electric kettle, set for steeping at 95° C. (actual temperature range of 199-200° F.=92.8-96.1° C.) for 1 hour before being decanted through different porosity filters for collection. All formulations were made in 500 mL volumes with TDC tap water.

Results: 10×RH—(211 g/L) Kettle overheated because liquid could not circulate—resulting product was a thick, sludgy porridge like consistency with very little free liquid. The resulting product could not be filtered through a coarse mesh sieve or coffee filter in any meaningful way. 5×RH—(105.5 g/L) Kettle heated as expected and mixture remained more flowable. Extract was filtered through coarse mesh sieve, large volume of solids remained behind, only retrieved ˜250 mL of liquid out of the original 500 mL once the solids were separated. Unable to filter mixture through coffee filter—milky opaque appearance with lots of suspended fine particles. Centrifuging a small sample at 15000×g for 5 min pelleted ˜25% as solids, 65% as a milky aqueous phase and ˜10% as a possible white lipid or cellulose (lighter than water) phase at the top. The aqueous phase was not filterable through a 0.22 μm syringe filter, despite the centrifugation. 2×RH—(42.2 g/L) Produced a very thick solution, but more flowable than the 5×. The bulk of the liquid was also difficult to separate, and the small particle density was too high for any of our filtration techniques. Many of the small partial rice particles left in the dry material had absorbed water and bulked up in this version. 1×RH—(21.1 g/L) Particulate matter from RH settles to the bottom of the vessel after standing, and separates into a solid phase and a decantable liquid phase.

### Alternative Preparation Methods.

Design: Trial investing alternative preparation methods including autoclaving RH diluent as an alternative to filter sterilization and cold steeping for extraction. Prepared three formulations for testing using TDC tap water.

Results: Formulation 1—1×RH (21 g/L) autoclaved at 121° C. for 30 minutes, cooled then placed in fridge overnight (˜14 hours). Three distinct layers/phases of the product emerged after the bottle adjusted to room temperature (Image 1). Upper phase was foamy and paler/white in color, floating on the surface of the solution. The upper phase was not homogenous and is present at different thicknesses around the circumference of the bottle. The upper phase mixes at an interface with the middle phase and did not have a clear border. Middle phase was turbid and beige with fine solids. This phase was pipetted without sticking or retention and filter sterilization was possible with smaller aliquots. Lower phase consisted of two portions, a layer of fine solids that have sedimented out and non-dissolved chunky solids and rice hull pieces at the bottom. The middle phase was drawn off using a serological pipette and filtered through a 5 μm and 1.2 μm filter. After filtration through the larger pore sizes, attempted to pass the filtrate through a 0.22 μm syringe filter, which clogged immediately. A 0.22 μm filter bottle was then tried, however the larger filtration surface area still clogged after only ˜25 mL.

Formulation 2—1×RH (21.1 g/L) RH were cold steeped 4° C. overnight (˜16 hours). Two noticeable phases were present in this formulation. Upper phase showed a gradient of dissolved solids. The upper part of the top phase was light beige and slightly translucent and the lower part of the top phase was beige comparatively more turbid—more similar to the middle phase of the autoclaved solution above. There was a slight white film at the top. Lower phase consisted of undissolved RH pieces and was more homogenous than the lower phase of the formulation 1. The cold steeped version was nearly entirely clarified overnight. 5 mL of supernatant was drawn off and filtered through a single 0.22 μm syringe filter rapidly.

Formulation 3—1×RH (21.1 g/L) was steeped at 95° C. for 1 hour, then cooled and placed in fridge overnight (14 hours). Two noticeable phases were present in this formulation, it was intermediate between the autoclaved and cold steeped version. Upper phase was a turbid beige with less translucence than the formulation 2 upper phase, but less color than the autoclaved version. Some foaminess and flecks of material stayed on the surface. Lower phase consisted of undissolved RH pieces and had a thinner layer of settled fine solids compared to formulation 1. The middle phase was drawn off using a serological pipette and filtered through a 5 μm and 1.2 μm filter as with the autoclaved version. After filtration through the larger pore sizes, the remainder was passed through 0.22 μm filter bottles, with a final yield of around 40 mL total from 80 mL sampled.

### Different Steeping Temperatures.

Purpose: Samples from the diluent preparation trials were analyzed via ion chromatography (IC) for ionic composition. The aim was to determine which extraction temperature of RH achieved the highest extracted ion content.

All samples were 0.22 μm filtered before submission using syringe filters and 10 mL syringes. Formulation 2 diluent filtered easily, the Formulation 1 and 3 had significantly more solids in suspension and required multiple filters to get the 10 mL of sample for analysis.

TABLE 25
Ion content (in PPM) of each RH formulation.

| | Formulation 2 | Formulation 3 | Formulation 1 |
|---|---|---|---|
| Chloride | 231.0 | 239.9 | 150.8 |
| Nitrite | 1.3 | — | 1.1 |
| Bromide | 6.8 | 6.8 | 6.7 |
| Nitrate | 7.7 | 7.8 | 8.3 |
| Phosphate | 21.9 | 40.5 | 101.6 |
| Sulfate | 20.0 | 22.6 | 18.8 |
| Lithium | 0.7 | 0.7 | 0.6 |
| Sodium | 99.8 | 105.7 | 66.6 |
| Ammonium | 2.0 | 2.0 | 3.8 |
| Potassium | 153.1 | 304.8 | 318.7 |
| Magnesium | 10.1 | 44.2 | 51.9 |
| Calcium | 8.8 | 10.5 | 6.2 |

### Continued Steeping.

Purpose: Two samples, Formulation 2 and Formulation 1, were analyzed for ion composition on IC following an additional 9 days of storage with the solid RH fraction. Extracts were stored at 4° C. in the fridge for 9 days following the first IC analysis to determine if any additional ions continued to dissolve into solution. The samples were treated with the same filtration protocol as described above.

TABLE 26
Ion content (in PPM) of each Formulation and test time point. Bold rows indicate
ions with a greater than 20% change in concentration between the two time points.

| | Formulation 2 | | | Formulation 1 | | |
|---|---|---|---|---|---|---|
| | Start | +9 DAYS | % Change | Start | +9 DAYS | % Change |
| Chloride | 231.0 | 241.8 | 4% | 150.8 | 244.8 | 38% |
| Nitrite | 1.3 | 0.0 | | 1.1 | 0.0 | |
| Bromide | 6.8 | 6.8 | 0% | 6.7 | 6.8 | 1% |
| Nitrate | 7.7 | 7.3 | −4% | 8.3 | 7.5 | −10% |
| Phosphate | 21.9 | 71.6 | 69% | 101.6 | 84.9 | −20% |
| Sulfate | 20.0 | 21.5 | 7% | 18.8 | 28.4 | 34% |
| Lithium | 0.7 | 1.3 | 49% | 0.6 | 1.0 | 42% |
| Sodium | 99.8 | 99.0 | −1% | 66.6 | 108.8 | 39% |
| Ammonium | 2.0 | 2.2 | 12% | 3.8 | 5.1 | 27% |
| Potassium | 153.1 | 198.6 | 23% | 318.7 | 310.1 | −3% |
| Magnesium | 10.1 | 21.3 | 53% | 51.9 | 55.3 | 6% |
| Calcium | 8.8 | 9.5 | 8% | 6.2 | 24.7 | 75% |

### Flask Culture Media Addition and Replacement.

Purpose: RH extract has the potential not only as a diluent but as a substrate for X. autotrophicus growth. The purpose of these experiments were to determine whether RH can replace the yeast extract in the fermentation media used to produce X. autotrophicus using MSDH process (4 g/L yeast extract) or enhance growth with a boosting effect. 4 g/L addition or substitution was chosen to mimic the amount of yeast extract (YE).

Design: Three treatments in biological duplicates (6 flasks) were prepared for this experiment. Each 250 mL flask was filled with 50 mL of treatment media, and inoculated targeting a 1E7 CFU/mL density with 50 μL of Biofertilizer. Flasks were started with and fed daily 100 mM methanol (190 μl) for the first 5 days. Day 6 was not sampled or fed, and day 7 the flasks were sampled and the experiment ended.

Cell concentrations were monitored over the experiment with daily CFU plating, in technical duplicates across a likely dilution range. Aliquots (1.5 ml) were collected daily for CFU plating and stored at 4° C. in case of additional testing requirements. Media pH and electrical conductivity (EC) were tested after preparation, values were recorded when analyte was at 27° C. (Table 27). Additionally, 500 μl samples were pulled from each flask at the final sampled time point for OD₆₀₀ PHB quantification using the established protocol. Samples were blanked with a pre-inoculation DO aliquot. Single samples were processed for each flask.

TABLE 27
pH and electroconductivity (mS/cm)
for each media before inoculation.

| Treatment | Growth Conditions | pH | EC |
|---|---|---|---|
| Control | Media 2.1 with no RH Added | 6.585 | 4.29 |
| Replacement | Media 2.1 with no YE, 4 g/L | 6.742 | 4.80 |
| | RH added | | |
| Boost | Media 2.1 with 4 g/L RH added | 6.578 | 4.42 |

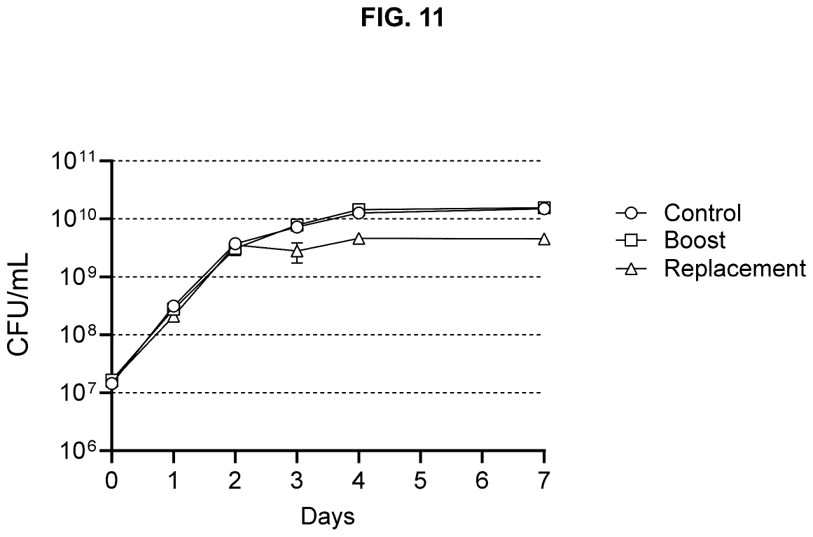

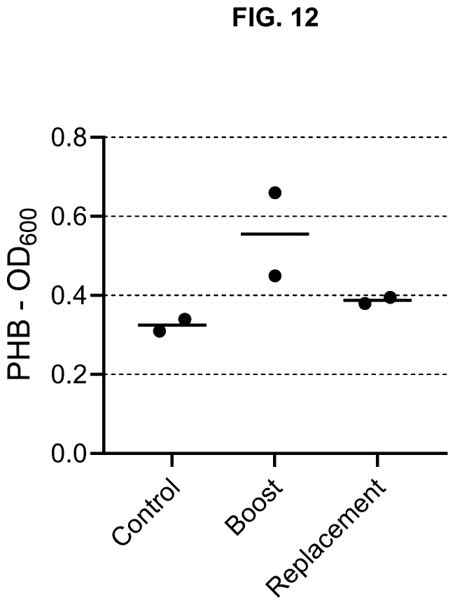

Results: Cultures grown in the RH “Boost” treatment aligned closely with the control treatment, while cultures in the RH “Replacement” treatment achieved ˜40% of cell density (CFU/ml) of the control treatment (FIG. 11). All OD₆₀₀ values read on the spectrophotometer were too low, out of the reliable reading range. However, values were still recorded and corrected for dilution (FIG. 12). Samples containing RH still had floating flecks of material after the bleach digestion that may interfere with absorbance measurements.

### Modified Flask Culture Media Addition and Replacement.

Design: To confirm findings, the flask culture media addition and replacement design was repeated with modifications including a reduced incubation time from 7 days to 4 days. Biological duplicate flasks were plated singly at each time point instead of technical duplicates. Additionally, flasks were not sampled or fed 100 mM MeOH on day 3.

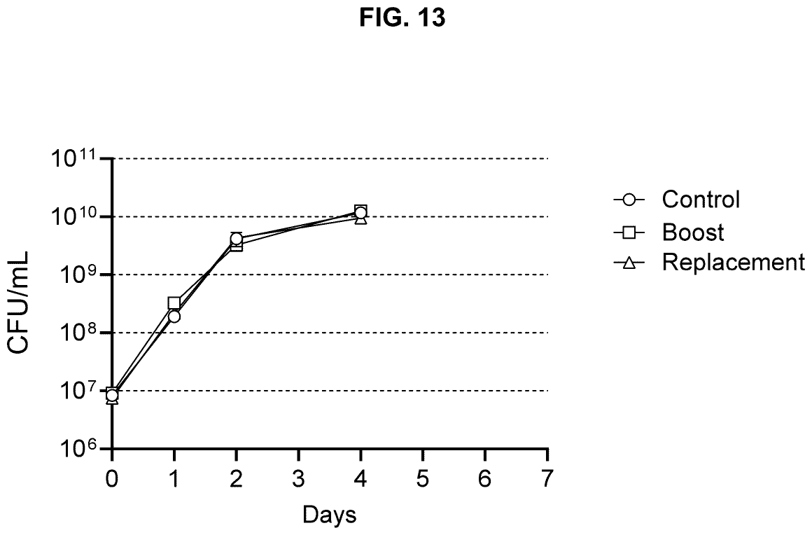

Results: Cultures grown in the RH “Boost” treatment aligned closely with the control treatment, while cultures in the RH “Replacement” treatment achieved ˜70% of cell density (CFU/ml) of the control treatment (FIG. 13).

### Diluent Compatibility.

Purpose: Autoclaved RH (Formulation 1) contained many dissolved ions that may affect X. autotrophicus survival when used as a diluent for storage. From diluent formulation trials above, the formulation 1 preparation method was chosen as optimal for workability and sterility. From this formulation, the middle phase (aqueous, without the floating white layer at the top and settled solids from the bottom) was used in diluent formulations, since it was homogenous and sterile.

Purpose: Identify an optimum RH concentration for supporting/maintaining cell viability in storage at 4° C. or room temperature.

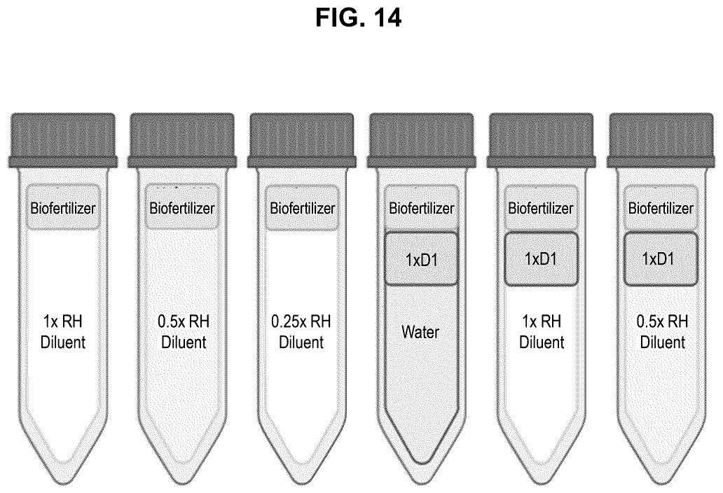

Design: Formulation 1 was used for all experiments, hereafter referred to as 1×D2 (21.1 g/L of rice hull). Liquid from middle phase of formulation 1 was removed with a serological pipette, vortexed, and then used for relevant treatments. Test samples were prepared using biofertilizer with cells only, which was produced from a bioreactor culture containing X. autotrophicus cells. Controls representing standard Biofertilizer+D1 were prepared using standard D1 diluent. All mixtures were prepared to a starting cell density of 1e9 CFU/ml. Five aliquots of 8 mL were prepared per treatment and stored in 15 mL aliquots in a refrigerator at 4° C. for 27 days. After 6 days a subset of samples were moved to room temperature (˜25° C.) and placed in a cabinet after plating, then sampled non-destructively repeatedly over 18 days. All samples from both storage temperatures were plated in triplicate at the −E6 dilution. See FIG. 14.

TABLE 28
Composition of Round 1 diluent trial treatments.

| | Treatment | 5 × D1 | 1 × D2 | DI H20 | X. autotrophicus | Total |
|---|---|---|---|---|---|---|
| Trt # | ID | (mL) | (mL) | (mL) | culture (mL) | (mL) |
| 1 | Biofertilizer + | 0 | 34.91 | 0 | 5.09 | 40 |
| | 1 × D2 | | | | | |
| 2 | Biofertilizer + | 0 | 17.45 | 17.45 | 5.09 | 40 |
| | 0.5 × D2 | | | | | |
| 3 | Biofertilizer + | 0 | 8.72 | 26.18 | 5.09 | 40 |
| | 0.25 × D2 | | | | | |
| 4 | Biofertilizer + | 6.98 | 27.92 | 0 | 5.09 | 40 |
| | 1 × D1 + 1 × D2 | | | | | |
| 5 | Biofertilizer + | 6.98 | 13.96 | 13.96 | 5.09 | 40 |
| | 1 × D1 + 0.5 × D2 | | | | | |
| 6 | Biofertilizer + D1 | 6.98 | 0 | 27.92 | 5.09 | 40 |

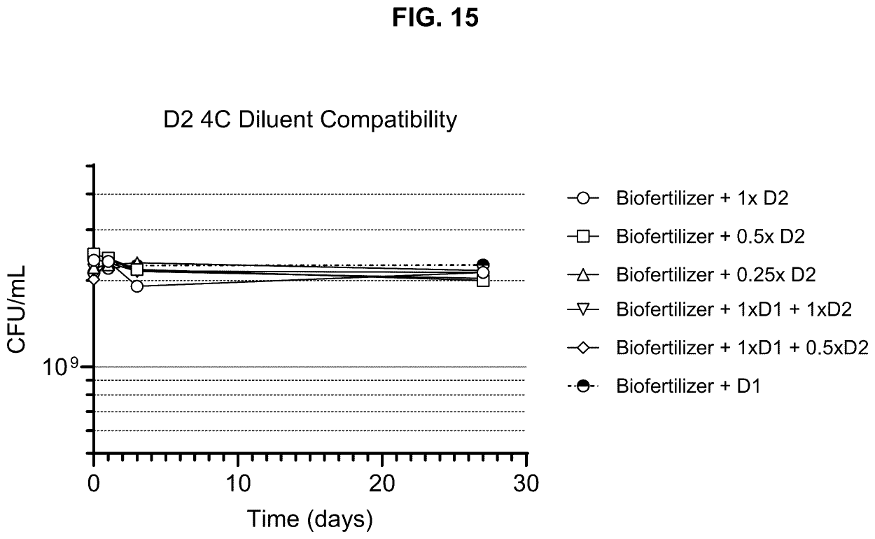

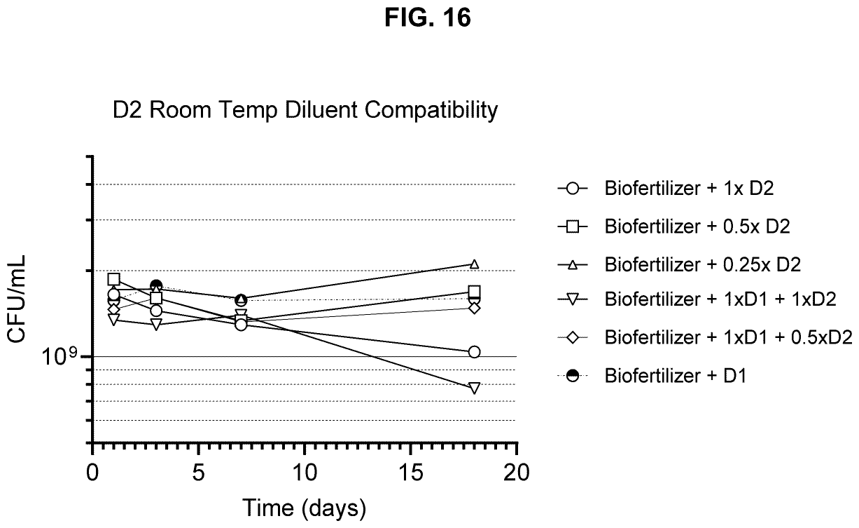

Results: At 4° C., there was little to no observable decline in CFUs over 21 days and no significant difference between standard Biofertilizer+D1 and test samples containing RH diluent (D2). Under room temperature conditions, lower concentrations of D2 maintained higher cell densities by the end of the trial at RT. Formulations containing 0.5× and 0.25×D2 had similar CFU counts at the end of the trial to the standard Biofertilizer+D1, while both formulations containing full strength (lx) D2 had reduced CFU counts. See FIGS. 15-16.

Purpose: Repeat best performing formulations at 25° C. Additional treatments including X. autotrophicus in water (Biofertilizer with cells only+water) as a control along with an additional diluent mixture (0.25×D2+1×D1) were also added.

Design: Formulation 1 was used for all experiments, hereafter referred to as 1×D2 (21.1 g/L of rice hull). Liquid from middle phase of formulation 1 was removed with a serological pipette, vortexed, and then used for relevant treatments. Test samples were prepared using biofertilizer with cells only and controls representing biofertilizer+D1 were prepared using standard D1 diluent. A single, non-destructive 5 mL aliquot was used for repeated sampling. Samples were plated in triplicate at the −E6 dilution with a single biological replicate. All samples were incubated at 25° C.

TABLE 29
Composition of diluent trial treatments

| | Treatment | 5 × D1 | 1 × D2 | DI H20 | X. autotrophicus | Total |
|---|---|---|---|---|---|---|
| Trt # | ID | (mL) | (mL) | (mL) | culture (mL) | (mL) |
| 1 | Biofertilizer + 0.25 × D2 | 0 | 23.26 | 69.79 | 6.95 | 100 |
| 2 | Biofertilizer + 0.5 × D2 | 0 | 46.53 | 46.53 | 6.95 | 100 |
| 3 | Biofertilizer + Water | 0 | 0 | 93.05 | 6.95 | 100 |
| 4 | Biofertilizer + | 18.61 | 23.26 | 51.18 | 6.95 | 100 |
| | 1 × D1 + 0.25 × D2 | | | | | |
| 5 | Biofertilizer + D1 | 18.61 | 0 | 74.44 | 6.95 | 100 |

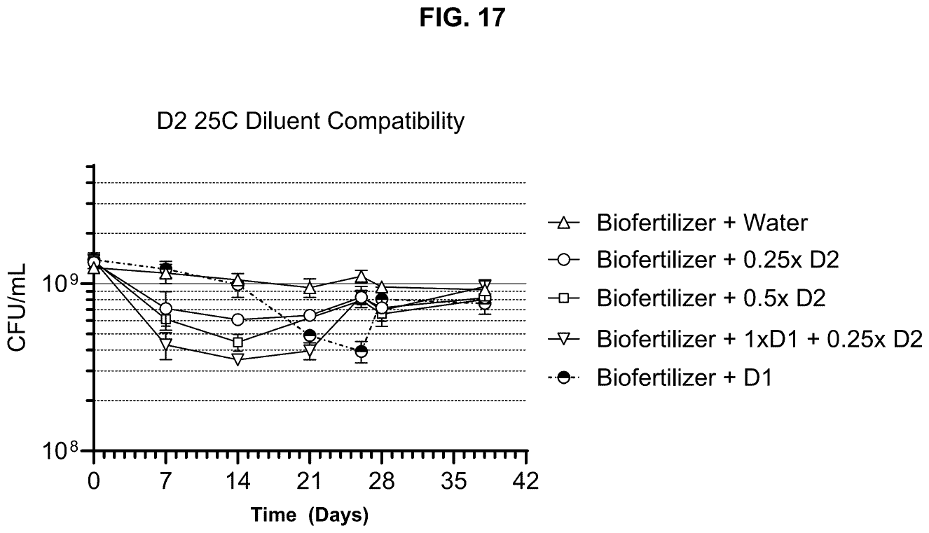

Results: All treatments converged on a similar CFU/mL at the end of the study period. Fluctuations observed in plate counts may be due to delayed growth after potentially stressful period at 25° C. More colonies appeared if plates were retained at room temperature for an additional 2 days and were added to the counts for the samples plated at day 28 and day 38, but not the previously plated samples. Day 28 samples were a replate of strange day 26 results. See FIG. 17.

### High Cell Density Trials.

Purpose: Test whether starting diluent treatment concentrations at a higher cell density impacts cell survival over time at 25° C. when using the most promising diluent candidate from previous trials, 0.25×D2.

Design: Formulation 1 was used for all experiments, hereafter referred to as 1×D2 (21.1 g/L of rice hull). Liquid from middle phase of formulation 1 was removed with a serological pipette, vortexed, and then used for relevant treatments. Test samples were prepared using X. autotrophicus bioreactor samples. Controls representing biofertilizer+D1 were prepared using standard D1 diluent. A single, non-destructive 10 mL aliquot was used for repeated sampling. Plating was performed in triplicate for each treatment, with a single biological replicate. Samples were stored at 25° C.

TABLE 30
Composition of higher density diluent trial treatments.

| | | 5 × D1 | 1 × D2 | DI H20 | biofertilizer | Total |
|---|---|---|---|---|---|---|
| Trt | Treatment | (mL) | (mL) | (mL) | (mL) | (mL) |
| 1 | Biofertilizer + | 1.81 | 0.00 | 7.25 | 0.94 | 10.00 |
| | D1 (1.5E9) | | | | | |
| 2 | Biofertilizer + | 1.96 | 0.00 | 7.85 | 0.19 | 10.00 |
| | D1 (1.5E9) | | | | | |
| 3 | Biofertilizer + | 0.00 | 1.72 | 5.16 | 3.13 | 10.00 |
| | 0.25 × D2 (5E9) | | | | | |
| 4 | Biofertilizer + | 0.00 | 2.34 | 7.03 | 0.63 | 10.00 |
| | 0.25 × D2 (5E9) | | | | | |
| 5 | Biofertilizer + | 0.00 | 2.19 | 6.56 | 1.26 | 10.00 |
| | 0.25 × D2 (1E10) | | | | | |

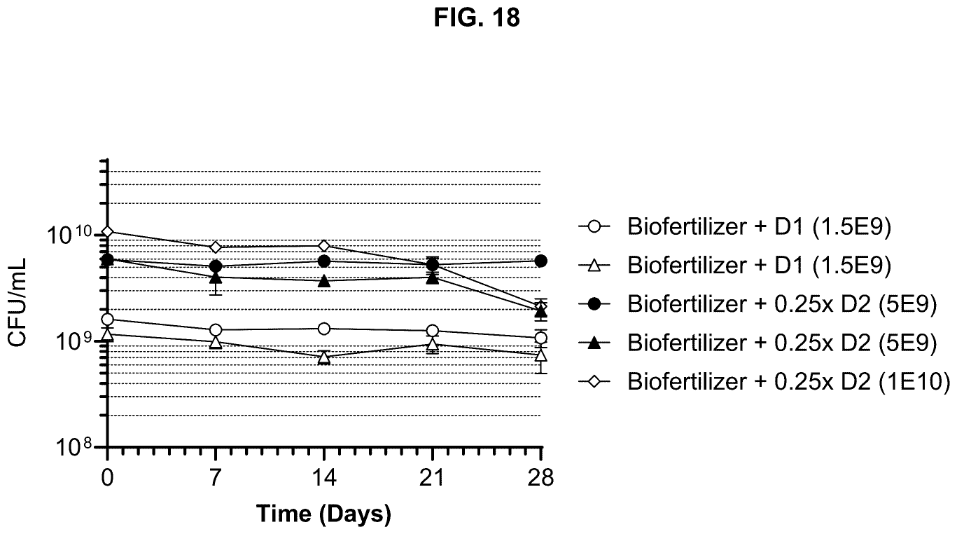

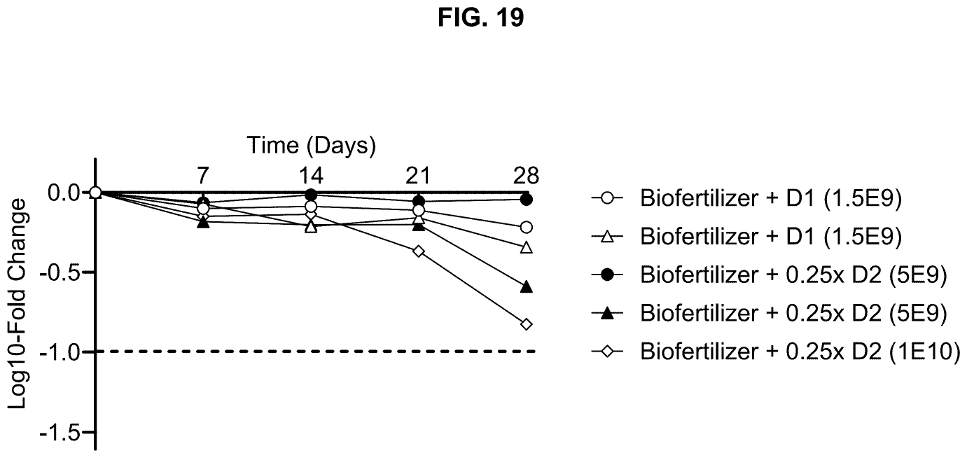

Results: High cell density (5E9 or 1E10 CFU/ml) treatments generally declined faster than lower cell density samples. The highest cell density 1E10 treatment declined the most overall by log-fold change. The Biofertilizer formulation with 0.25×D2 did not decline over time, showing similar stability to standard Biofertilizer+D1. See FIG. 18 and FIG. 19.

### Lettuce Tissue Culture Trials.

Purpose: An ideal diluent for X. autotrophicus product would not only provide a storage medium but increase plant growth through some mechanism—either on its own or synergistically. For RH to be a viable diluent candidate it needs to be at least as efficacious as or better than D1 in storage and plant growth promotion or pose some other benefit. Combinatorial treatments of D1 and D2 may behave differently than either diluent alone when combined with X. autotrophicus biofertilizer. Test how RH diluent and formulations perform when compared to the Biofertilizer+D1 and controls in lettuce seedling tissue culture vessel plant trials.

Design: Eight replicate Magenta™ Vessels per treatment were prepared for tissue culture with 100 mL of 50% concentration Hoagland's nutrient, 0.8% agar, with 2.5 g/L glucose. Romaine Lettuce seeds (Lactuca sativa var. salivus, Johnny's Selected Seeds, Winslow, Maine, USA) were surface sterilized and stratified at 4° C. for 48 hours prior to germination on 50% Hoagland's nutrient agar plates stored in a growth chamber with 16-hour day/8-hour night light cycles, temperatures at 25° C. during the day and 22° C. at night, and humidity maintained at 50%. Seeds were germinated for only 3 days compared to the average 4 days prior to transplant. Seedlings with root lengths of approximately 1 cm were transplanted into the Magenta™ Vessels.

Treatment media was prepared with biofertilizer and D1 diluent or Formulation 1 D2 and applied to seedlings transplanted into Magenta™ vessels in 10 μL volumes. All treatment media formulations, other than the negative control, were made to a calculated 1.5E9 CFU/mL density. Seedlings were grown for 3 weeks in a growth chamber with 16-hour day/8-hour night light cycles, temperatures at 25° C. during the day and 22° C. at night, and humidity maintained at 50% before being harvested for fresh vegetative biomass measurements, excluding roots. Treatments and calculations for stock preparations are as described in Table 31.

TABLE 31
Treatment composition for lettuce trials.

| | | 5 × | 1 × D2 | Tap H20 | biofertilizer | Total |
|---|---|---|---|---|---|---|
| Trt | Treatment | D1(mL) | (mL) | (mL) | (mL) | (mL) |
| 1 | 50% Hoagland's control | — | — | 40 | — | 40 |
| 2 | Biofertilizer + | 7.44 | — | 29.77 | 2.78 | 40 |
| | D1 Control | | | | | |
| 3 | Biofertilizer with | — | — | 37.21 | 2.78 | 40 |
| | cells only Control | | | | | |
| 4 | 1 × D1 only | 8 | — | 32 | — | 40 |
| 5 | 1 × D2 only | — | 40 | — | — | 40 |
| 6 | Biofertilizer + | — | 37.21 | — | 2.78 | 40 |
| | 1 × D2 | | | | | |
| 7 | Biofertilizer + | 7.44 | 29.77 | — | 2.78 | 40 |
| | 1 × D1 + 1 × D2 | | | | | |

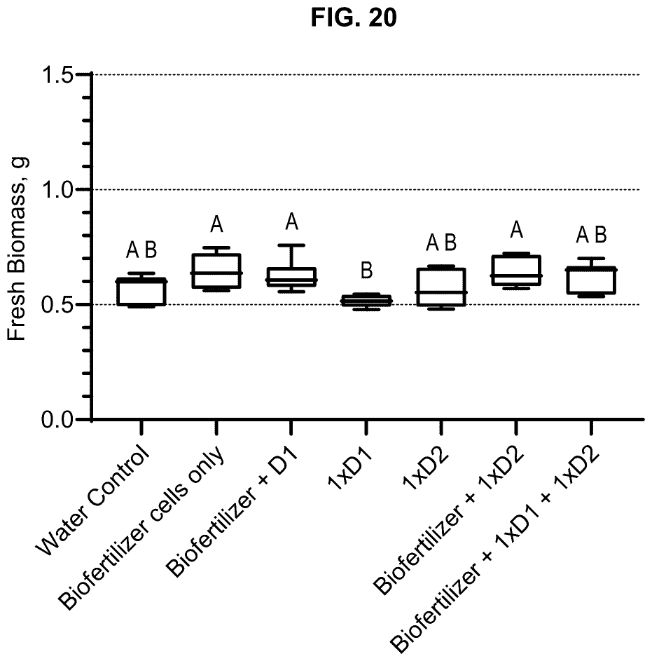

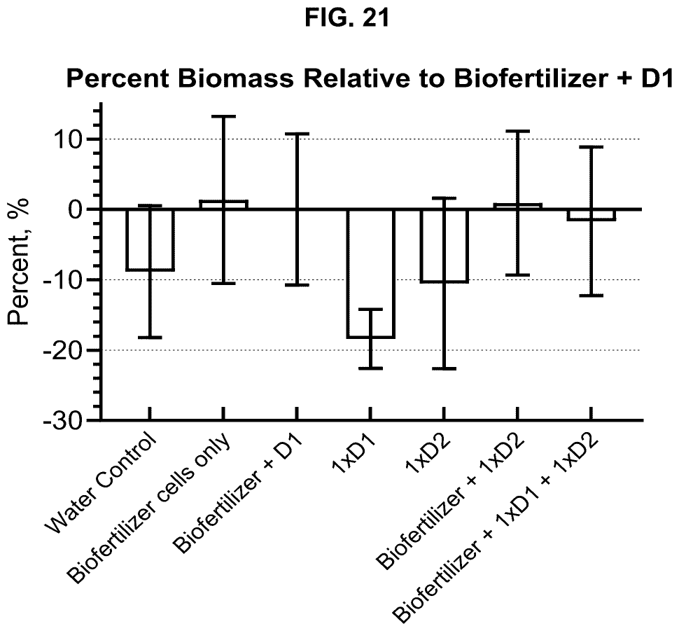

Results: Results show no statistically significant differences in biomass between the control seedling biomass and the Biofertilizer+D1 or Biofertilizer with cells only control treatments. No significantly negative effects of D2 on plant biomass were recognized in this experiment. Potential reasons for this result could be due to issues with seed health and germination. Following this experiment, lettuce seeds germinated from the same lot were tested, and had a low germination rate (approximately 50% compared to typical 90%) and the leaves of germinated seedlings showed unusual browning along the cotyledon mid ribs. See FIG. 20 and FIG. 21. Experiment was refined based on diluent trials and repeated.

### Redefined Lettuce Tissue Culture Trials.

Design: Ten replicate Magenta™ Vessels per treatment were prepared for tissue culture with 100 mL of 50% concentration Hoagland's nutrient, 0.8% agar, with 2.5 g/L glucose. Bromothymol blue was incorporated into the media starting with round 2, a color change indicator that turns blue as the agar media becomes more basic. X. autotrophicus' nitrogen fixation pathway is hypothesized to drive this change, and it can be used as an early indicator of nitrogen fixation activity before harvest.

Romaine Lettuce seeds (Lactuca sativa var. salivus, Johnny's Selected Seeds, Winslow, Maine, USA) were surface sterilized and stratified at 4 C. for 48 hours prior to germination on 50% Hoagland's nutrient agar plates incubated in a growth chamber with 16-hour day/8-hour night light cycles, temperatures at 25° C. during the day and 22° C. at night, and humidity maintained at 50% 3 days. Seeds were germinated for 96 hours. Seedlings with root lengths of approximately 1-1.5 cm were transplanted into the Magenta™ Vessels.

Treatment media was prepared with biofertilizer and D1 diluent or Formulation 1 D2 and applied to seedlings transplanted into Magenta™ vessels in 10 μL volumes. All treatment media formulations, other than the negative control, were made to a calculated 1.5E9 CFU/mL density. Seedlings were grown for 3 weeks in a growth chamber with 16-hour day/8-hour night light cycles, temperatures at 25° C. during the day and 22° C. at night, and humidity maintained at 50% before being harvested for fresh vegetative biomass measurements, excluding roots. Treatments and calculations for stock preparations are as described in Table 32.

TABLE 32
Composition of redefined lettuce trial treatments.

| | | 5 × D1 | 1 × D2 | Tap H20 | biofertilizer | Total |
|---|---|---|---|---|---|---|
| Trt | Treatment | (mL) | (mL) | (mL) | (mL) | (mL) |
| 1 | 50% Hoagland's control | — | — | — | — | — |
| 2 | Biofertilizer + | 2 | — | 7.19 | 0.81 | 10 |
| | D1 Control | | | | | |
| 3 | Biofertilizer cells | — | — | 9.19 | 0.81 | 10 |
| | only Control | | | | | |
| 4 | Biofertilizer + | — | 9.19 | — | 0.81 | 10 |
| | 1 × D2 | | | | | |
| 5 | Biofertilizer + | — | 4.60 | 4.60 | 0.81 | 10 |
| | 0.5 × D2 | | | | | |
| 6 | Biofertilizer + | — | 2.30 | 6.89 | 0.81 | 10 |
| | 0.25 × D2 | | | | | |

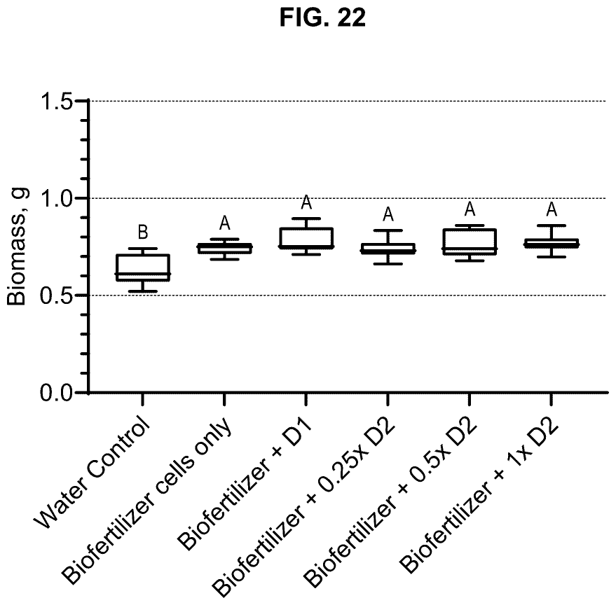

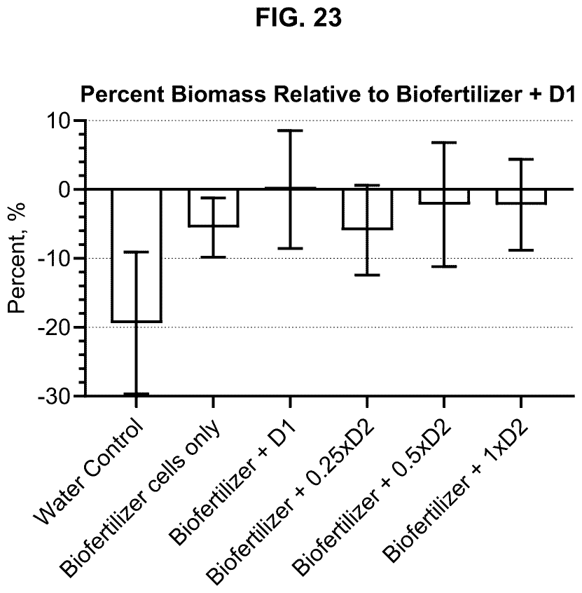

Results: Biofertilizer with cells only and Biofertilizer+D1 control treatments have statistically greater average biomasses compared to the average biomass of the Water Control treatment in this trial demonstrating the trial performed as expected. Biomass from treatments containing RH diluent (D2) were statistically similar to the Biofertilizer+D1 control treatment. See FIG. 22 and FIG. 23.

### Lower Density X. Autotrophicus in Lettuce Trials.

Design: Nine replicate Magenta™ Vessels per treatment were prepared for tissue culture with 100 mL of 50% concentration Hoagland's nutrient, 0.8% agar, with 2.5 g/L glucose. Bromothymol blue was incorporated into the media starting with round 2, a color change indicator that turns blue as the agar media becomes more basic. X. autotrophicus' nitrogen fixation pathway is hypothesized to drive this change, and it can be used as an early indicator of nitrogen fixation activity before harvest.

Romaine Lettuce seeds (Lactuca sativa var. salivus, Johnny's Selected Seeds, Winslow, Maine, USA) were surface sterilized and stratified at 4° C. for 48 hours prior to germination on 50% Hoagland's nutrient agar plates incubated in a growth chamber with 16-hour day/8-hour night light cycles, temperatures at 25° C. during the day and 22° C. at night, and humidity maintained at 50% for 3 days. Seeds were germinated for 96 hours. Seedlings with root lengths of approximately 1-1.5 cm were transplanted into the Magenta™ vessels.

Treatment media was prepared with biofertilizer and D1 diluent or Formulation 1 D2 and applied to seedlings transplanted into Magenta™ vessels in 10 μL volumes. Seedlings were grown for 3 weeks in a growth chamber with 16-hour day/8-hour night light cycles, temperatures at 25° C. during the day and 22° C. at night, and humidity maintained at 50% before being harvested for fresh vegetative biomass measurements, excluding roots. Treatments and calculations for stock preparations are as described in Table 33.

TABLE 33
Composition of lower density treatments.

| | | 5 × D1 | 1 × D2 | Tap H20 | biofertilizer | Total |
|---|---|---|---|---|---|---|
| Trt | Treatment | (mL) | (mL) | (mL) | (mL) | (mL) |
| 1 | 50% Hoagland's control | — | — | — | — | — |
| 2 | Biofertilizer + | — | — | 9.58 | 0.42 | 10 |
| | D1 Control | | | | | |
| 3 | Biofertilizer with | 1.92 | — | 7.66 | 0.42 | 10 |
| | cells only Control | | | | | |
| 4 | Biofertilizer + | — | 2.40 | 7.19 | 0.42 | 10 |
| | 0.25 × D2 | | | | | |
| 5 | Biofertilizer + | — | 4.79 | 4.79 | 0.42 | 10 |
| | 0.5 × D2 | | | | | |
| 6 | Biofertilizer + | 1.92 | 1.92 | 5.75 | 0.42 | 10 |
| | 1 × D1 + 0.25 × D2 | | | | | |

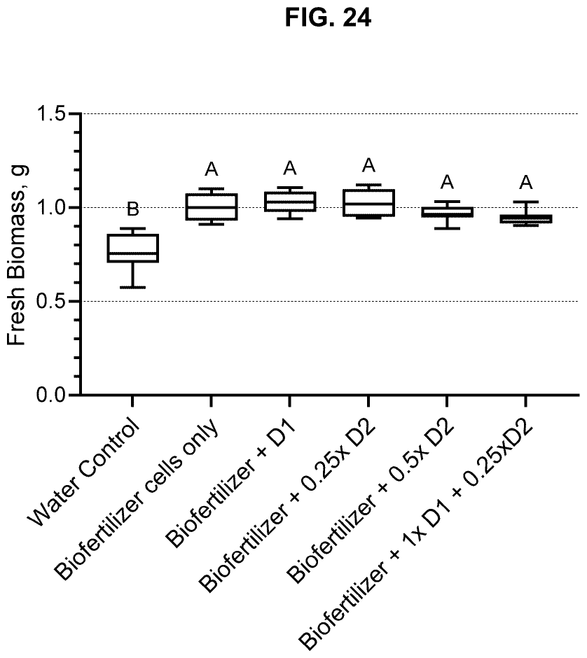

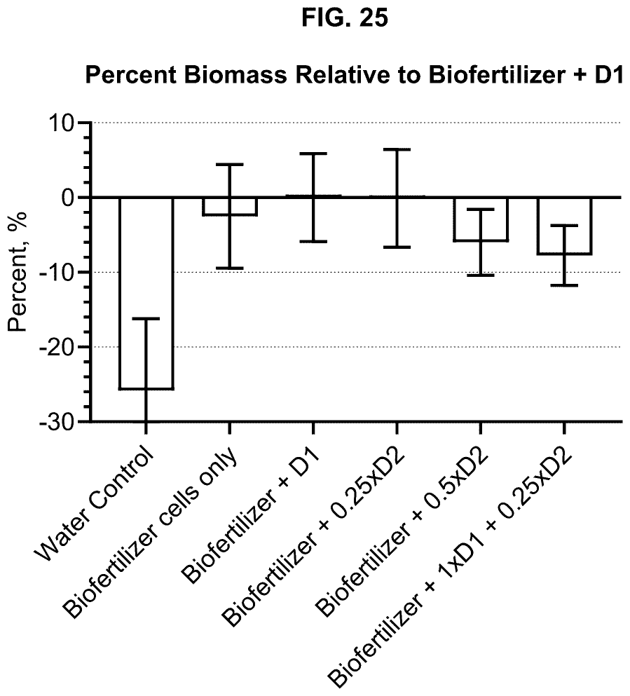

Results: Biofertilizer with cells only and Biofertilizer+D1 Control treatments have statistically greater average biomasses compared to the average biomass of the Water Control treatment in this trial. Biomass from treatments containing RH diluent (D2) were statistically similar to the Biofertilizer+D1 control treatment. See FIG. 24 and FIG. 25.

### Example 9—Combined Microorganisms and Diluent

The purpose of this experiment was to test efficacy of combined microorganisms with diluent for enhancing lateral root development by microbial fertilizers. Lateral root development was assessed in romaine lettuce seedlings (Lactuca sativa var. salivus, Johnny's Selected Seeds, Winslow, Maine, USA) as follows. 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach. Seeds were exposed to bleach solution for 10 minutes after which the bleach solution was aspirated, and the seeds were washed with 1 ml of sterile water six times. Seeds were then resuspended 1 ml sterile water and placed at 4° C. for 48 hours to stratify after which the water was aspirated, and seeds were stored dry at 4° C. for up to 2 weeks before sowing. Sterile lettuce seeds were placed near the top of the 50% Hoagland's agar plate along the “sowing line”. Using a pipette, 30 μl of either water (negative control) or tested product (according to Table 34 below), were plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at a ˜80° angle during growth to encourage root growth along the agar surface. Seedlings were grown for 7 days and then root development was assessed as lateral root density determined by dividing the lateral root number of total tap root length.

In this experiment, Standard Biofertilizer containing bioreactor grown X. autotrophicus was used as a positive control. For all microbial mixtures, bacteria including X. autotrophicus, Pseudomonas fluorescens, Rhodopseudomonas palustris, Azospirillum lipoferum, Cupriavidus necator were grown in flasks of nutrient broth (NB) in a shaking incubator at 30° C. and 200 rpm. Flasks were grown for 24 hours and then cultures were adjusted to a cell density of 1e9 CFU/ml based on an OD₆₀₀ of 0.57. Adjusted cultures were then used to prepare test samples as described above.

TABLE 34
Lateral root development in lettuce treatments.

| Treatment | | | |
|---|---|---|---|
| # | Treatment Description | Diluent Tested | Microbe Tested |
| 1 | Negative control (Water Only) | — | — |
| 2 | Biofertilizer + D1 | Alfalfa (21 g/L) | X. autotrophicus (1e9 CFU/ml) |
| 3 | Xa + Diluent | Alfalfa (21 g/L) | X. autotrophicus (1e9 CFU/ml) |
| 4 | Xa + Pf + Diluent | Alfalfa (21 g/L) | X. autotrophicus (5e8 CFU/ml) + |
| | | | P. fluorescens (5e8 CFU/ml) |
| 5 | Xa + Rp + Diluent | Alfalfa (21 g/L) | X. autotrophicus (5e8 CFU/ml) + |
| | | | R. palustris (5e8 CFU/ml) |
| 6 | Xa + Al + Diluent | Alfalfa (21 g/L) | X. autotrophicus (5e8 CFU/ml) + |
| | | | A. lipoferum (5e8 CFU/ml) |
| 7 | Xa + Cn + Diluent | Alfalfa (21 g/L) | X. autotrophicus (5e8 CFU/ml) + |
| | | | C. necator (5e8 CFU/ml) |

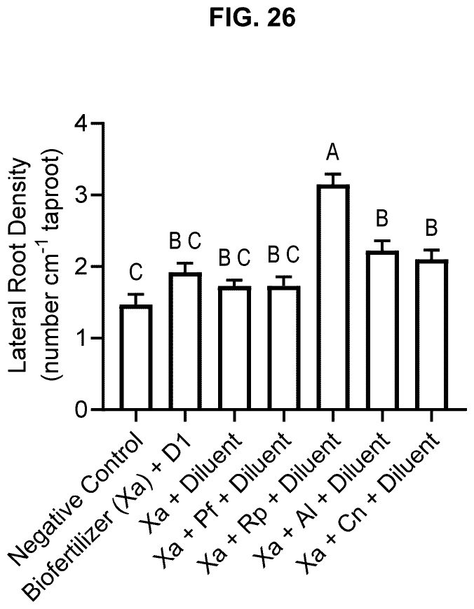

Application of Biofertilizer+D1 increased lateral root density of lettuce seedlings compared to the negative control (FIG. 26). This expected result validates the outcomes of this assay. Application of Biofertilizer prepared with tomato or orange peel extract increased lateral root density compared to the negative control regardless of diluent concentration. However, only tomato extract at 0.218 g/L and orange peel extract at 2.198 g/L significantly increased lateral root density compared to the negative control.

### Example 10—Freeze Dried Fermentate with Liquid Diluents

Purpose: To investigate and confirm the efficacy of dry product formulations using four distinct experimental approaches.

Across the four experiments listed below dry products were prepared for application to plants in a range of ways. Below is a treatment key describing how each version was prepared.

Freeze Dry Formulation V1: V1 uses a freeze dried fermentate with a liquid diluent. V1 is prepared by first resuspended the freeze dried fermentate powder in water before combining with diluent. This is then applied as a standard liquid product at ˜1e9 CFU/ml.

Freeze Dry Formulation V1—cells only: V1—biofertilizer with cells only used a freeze dried fermentate. V1—biofertilizer with cells only is prepared by resuspending the freeze dried fermentate in water before applying directly to plants as a liquid at ˜1e10 CFU/ml. Diluent is not included in this version.

Freeze Dry Formulation V2: V2 uses a freeze dried biofertilizer+D1 which includes fermentate and diluent dried together. V2 is prepared by resuspending the freeze dried finished product in water before applying directly to plants as standard liquid product at ˜1e9 CFU/ml.

Freeze Dry Formulation V3: V3 uses a freeze dried fermentate and freeze dried diluent which are dried separately. V3 is prepared by resuspending the freeze dried fermentate in water and resuspending the freeze dried diluent in water. Resuspended fermentate and diluent are then combined to prepare standard liquid product. This is applied to plants as standard liquid product at ˜1e9 CFU/ml.

Freeze Dry Formulation V4: V4 uses a liquid fermentate and a freeze dried diluent. V4 is prepared by first resuspending the freeze dried diluent in water and then combining with liquid fermentate to prepare standard liquid product. This is applied to plants as standard liquid product at ˜1e9 CFU/ml.

### Lateral Root Assay.

The purpose of this experiment was to investigate the ability of dry Biofertilizer formulation to enhance root development of lettuce seedlings compared to the standard liquid Biofertilizer. Lateral root development was assessed in romaine lettuce seedlings (Lactuca sativa var. salivus) as follows. 50% Hoagland's media prepared with 1.5% agar was adjusted to pH 6.5 and poured into 100 mm×100 mm square plates. Lettuce seeds were prepared for plating by first surface sterilizing and stratifying as follows: a 2 ml sterile microcentrifuge tube was filled with 500 μl of seeds along with 1 ml of 50% bleach. Seeds were exposed to bleach solution for 10 minutes after which the bleach solution was aspirated, and the seeds were washed with 1 ml of sterile water six times. Seeds were then resuspended 1 ml sterile water and placed at 4° C. for 48 hours to stratify after which the water was aspirated, and seeds were stored dry at 4° C. for up to 2 weeks before sowing. Sterile lettuce seeds were placed near the top of the 50% Hoagland's agar plate along the “sowing line”. Using a pipette, 30 μl of either water (negative control) or tested product (according to Table 35 below), were plated in a single line 45 mm below the sowing line. Plates were then placed in a growth chamber with 16-hour day/8-hour night light cycles, temperatures at 25° C. during the day and 22° C. at night, and humidity maintained at 50%. Plates were kept at a ˜80° angle during growth to encourage root growth along the agar surface. Seedlings were grown for 7 days and then root development was assessed as lateral root density determined by dividing the lateral root number of total tap root length.

TABLE 35
Treatments in the Lateral Root Assay

| | | Biofertilizer | Diluent |
|---|---|---|---|
| Treatment # | Treatment Description | Formulation | Formulation |
| 1 | Negative Control | — | — |
| 2 | Biofertilizer + D1 | Liquid | Liquid |
| 3 | Freeze Dry Formulation V1 − | Dry | — |
| | cells only | | |
| 4 | Freeze Dry Formulation V1 | Dry | Liquid |
| 5 | Freeze Dry Formulation V3 | Dry | Dry |
| 6 | Freeze Dry Formulation V4 | Liquid | Dry |

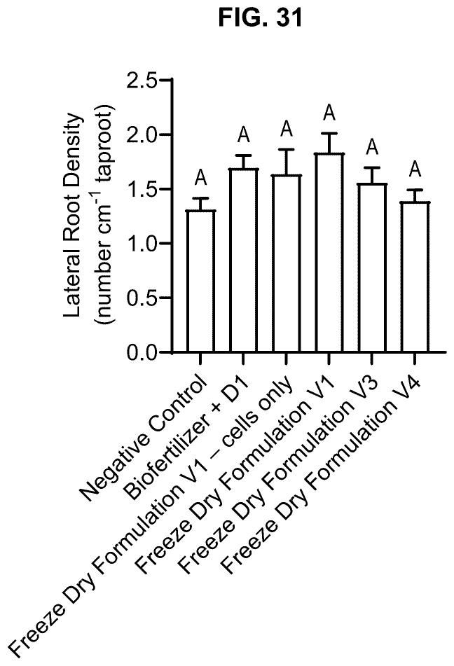

Application of Biofertilizer+D1, Freeze Dry Formulation V1—cells only, Freeze Dry Formulation V1, and Freeze Dry Formulation V3 all increased lateral root density of lettuce seedlings compared to the negative control (FIG. 31). Application of Freeze Dry Formulation V1 resulted in the greatest lateral root density. These results demonstrate root development of lettuce is similarly enhanced by standard liquid product and tested dry formulations.

### Tissue Culture Assay.

The purpose of this experiment was to investigate the efficacy of a dry Biofertilizer formulation relative to the standard liquid Biofertilizer+D1 product on lettuce under optimized growth conditions. Romaine lettuce seedlings (Lactuca sativa) were surface sterilized as described in experiment #1 above. Sterile seeds were plated onto 50% Hoagland's agar plates and germinated for 5 days before transplanting to tissue culture systems. Seedlings were transplanted into tissue culture vessels containing 50% Hoagland's agar. Seedlings were then treated with 10 μl of water or 10 μl of product according to table 36 below. This volume of product delivered 1e7 CFU of microbial cells per plant. Plants were grown for 21 days in a growth chamber with 16-hour day/8-hour night light cycles, temperatures at 25° C. during the day and 22° C. at night, and humidity maintained at 50% before harvesting for aboveground fresh biomass.

TABLE 36
Treatments in the Tissue Culture Assay

| | | Biofertilizer | Diluent |
|---|---|---|---|
| Treatment # | Treatment Description | Formulation | Formulation |
| 1 | Negative Control | — | — |
| 2 | Biofertilizer + D1 | Liquid | Liquid |
| 3 | Freeze Dry Formulation V1 − | Dry | — |
| | cells only | | |
| 4 | Freeze Dry Formulation V1 | Dry | Liquid |

| | | |
|---|---|---|
| 5 | Freeze Dry Formulation V2 | Dry |

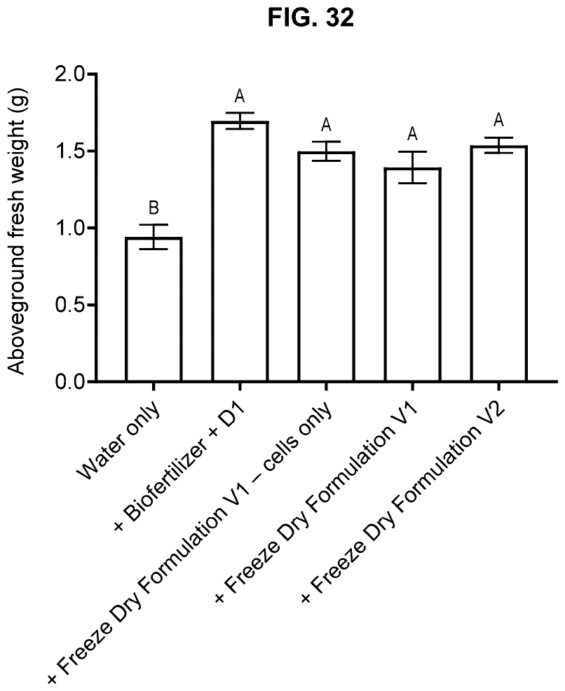

Application of Biofertilizer+D1, Freeze Dry Formulation V1—cells only, Freeze Dry Formulation V1, and Freeze Dry Formulation V2 all significantly increased aboveground biomass of lettuce compared to application of water only in a 50% Hoagland growth environment (FIG. 32). These results demonstrate a similar potential for Freeze Dry formulations and Biofertilizer+D1 standard liquid product to enhance lettuce growth under tissue culture conditions.

### Lettuce Pot Trial.

The purpose of this experiment was to investigate the efficacy of a dry Biofertilizer formulation relative to the standard liquid Biofertilizer+D1 product on lettuce. Romaine lettuce seedlings (Lactuca sativa) are sown into trays containing sunshine mix #1. Once germinated, seedlings are watered as needed and provided weekly doses of 50% Hoagland's solution with nitrogen to support growth. Seedlings are grown for 21-days before transplanting for the experimental trial. Seedlings are transplanted into 4″ pots containing coconut coir potting media. Lettuce plants are grown for 28 days from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 80% grower standard practice (GSP) or 100% GSP according to the treatments listed in Table 37 below. Biofertilizer (liquid or dry formulation) was applied as described below. Plants are watered as needed during the growth period. After 28 days of growth, lettuce plants are harvested for aboveground fresh weight and leaf tissue nitrogen (N). Leaf tissue nitrogen (%) was used to determine total leaf nitrogen by multiplying percent nitrogen and aboveground dry biomass. Nitrogen use efficiency crop (NUE_{crop}) was calculated as total leaf nitrogen divided by nitrogen supplied as fertilizer.

TABLE 37
Lettuce Pot Trial Treatments

| | | N equiv. | Biofertilizer | |
|---|---|---|---|---|
| Treatment # | Treatment Description | (lb/ac) | Formulation | Rate |
| 1 | 80% GSP | 36 | — | — |
| 2 | 80% GSP + Biofertilizer + D1 | 36 | Liquid | 8 oz/ac |
| 3 | 80% GSP + Freeze Dry Formulation V1 | 36 | Dry | 8 oz/ac |
| 4 | 80% GSP + Freeze Dry Formulation V2 | 36 | Dry | 8 oz/ac |
| 5 | 100% GSP | 48 | — | — |

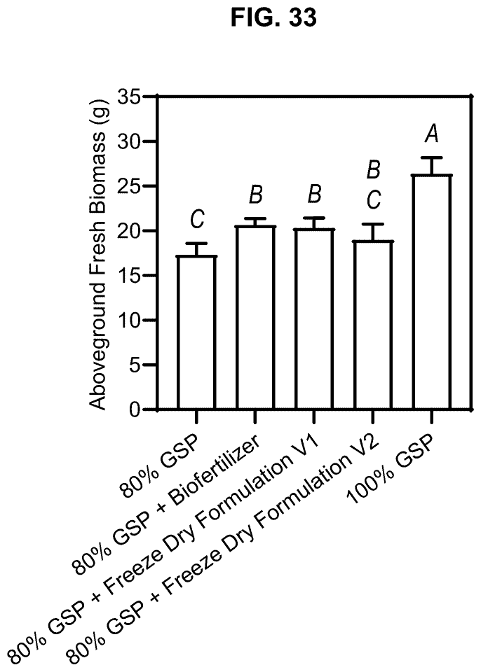

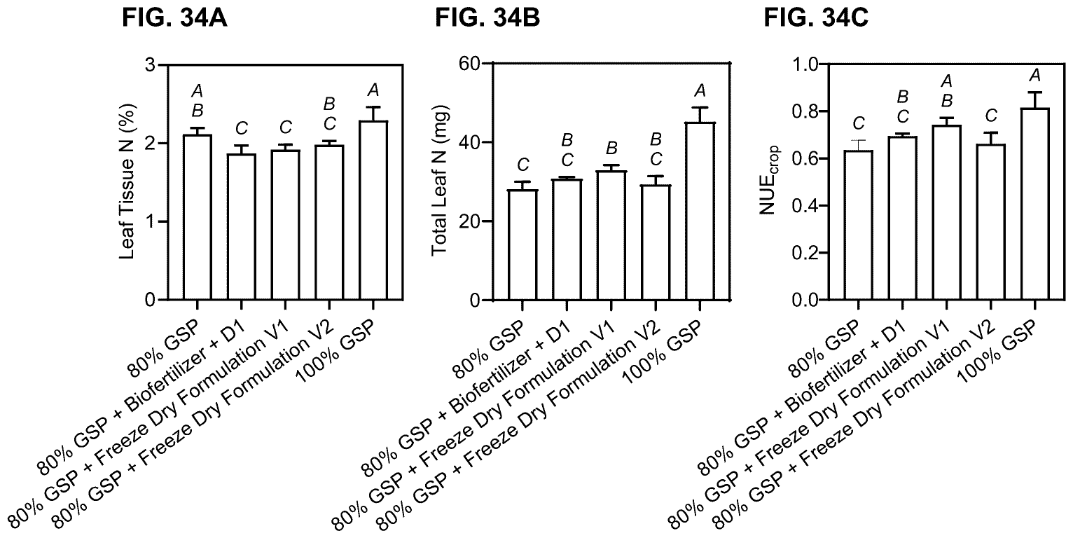

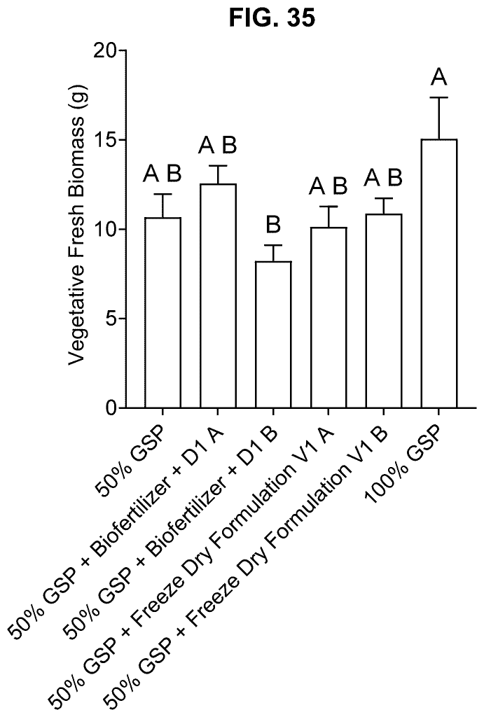

Application of Biofertilizer+D1 and Freeze Dry Formulation V1 significantly increased aboveground biomass of lettuce compared to the low nitrogen control of 80% GSP (FIG. 33). The application of Freeze Dry Formulation V2 did not significantly increase aboveground biomass of lettuce compared to the 80% GSP treatment. Percent leaf tissue N was lower in all biofertilizer treatments compared to the 80% GSP (FIGS. 34A-34C). However, total leaf N and NUE_{crop} was greater in the Biofertilizer+D1 and Freeze Dry Formulation V1 treatments compared to the 80% GSP (FIG. 35). These results demonstrate similar efficacy of Biofertilizer+D1 and Freeze Dry Formulation V1 on lettuce indicating that freeze dried fermentate can be successfully resuspended without efficacy loss.

### Tomato Pot Trial

The purpose of this experiment was to investigate the efficacy of a dry biofertilizer formulation relative to the standard liquid Biofertilizer+D1 product on tomatoes. Micro tomato seedlings (Solanum lycopersicum) were sown into trays containing sunshine mix #1. Once germinated, seedlings are watered as needed and provided weekly doses of 50% Hoagland's solution with nitrogen to support growth. Seedlings are grown for 40 days before transplanting for the experimental trial. Seedlings are transplanted into 4″ pots containing coconut coir potting media. Tomato plants are grown for 8 weeks from transplant to harvest in a growth chamber with 16-hour day/8-hour night light cycles, temperatures at 25° C. during the day and 22° C. at night, and humidity maintained at 50%. During the growth period, plants received weekly doses of nitrogen fertilizer as UAN-32 and nitrogen free Hoagland's solution for micronutrients. Nitrogen fertilizer was applied at 50% GSP or 100% GSP according to the treatments listed in Table 38 below. Biofertilizer (liquid or dry formulation) was applied using two different volumes of water for delivery as described in Table 38 below. Plants are watered as needed during the growth period. After 8 weeks of growth, lettuce plants are harvested for aboveground vegetative biomass and fruit yield. Fruit yields were recorded as total mass of fruit per plant, total mass of red fruit per plant, and total fruit counts per plant. Average fruit size was calculated by dividing total fruit mass per plant by total fruit counts per plant.

TABLE 38
Treatments for Tomato Pot trial

| | | | Biofertilizer | | |
|---|---|---|---|---|---|
| | | | | | Water |
| Treatment | | N equiv. | | | delivery |
| # | Treatment Description | (lb/ac) | Formulation | Rate | volume |
| 1 | 50% GSP | 100 | — | — | — |
| 2 | 50% GSP + Biofertilizer + D1 (A) | 100 | Liquid | 8 oz/ac | 20 ml |
| 3 | 50% GSP + Biofertilizer + D1 (B) | 100 | Liquid | 8 oz/ac | 40 ml |
| 4 | 50% GSP + Freeze Dry Formulation V1 (A) | 100 | Dry | 8 oz/ac | 20 ml |
| 5 | 50% GSP + Freeze Dry Formulation V1 (B) | 100 | Dry | 8 oz/ac | 40 ml |
| 6 | 100% GSP | 200 | — | — | — |

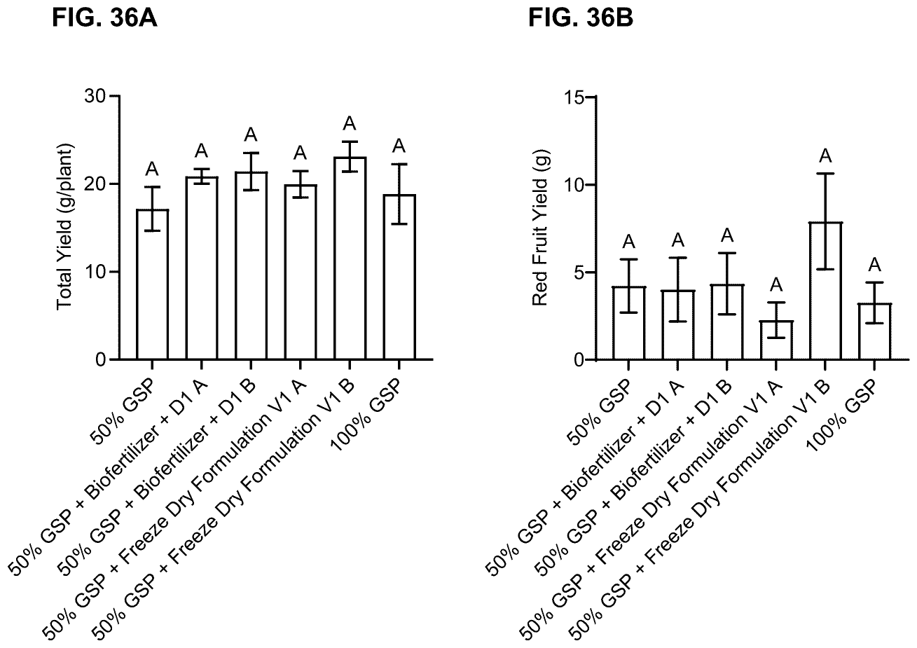

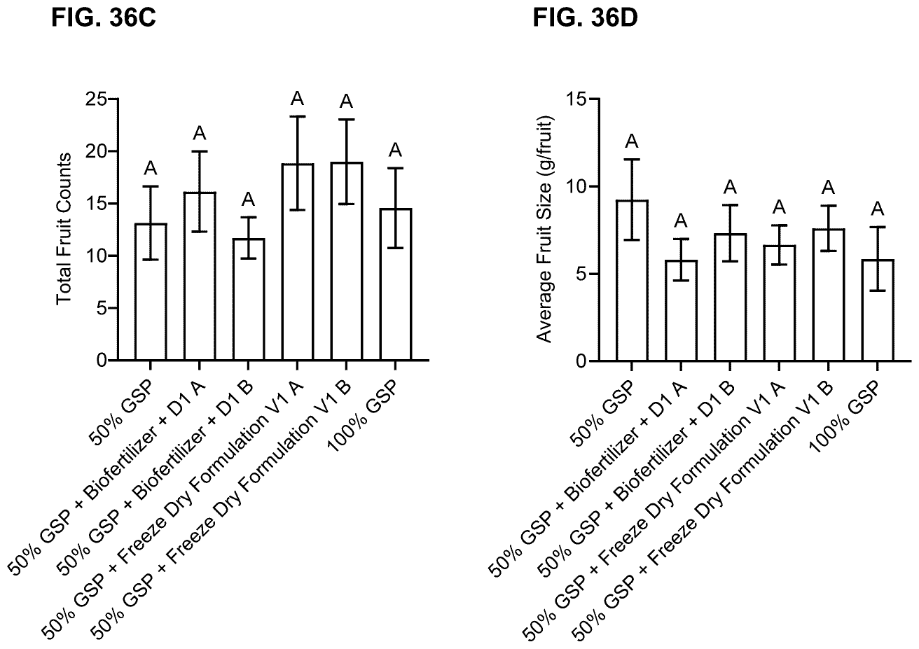

Application of Biofertilizer+D1 and Freeze Dry Formulation V1, regardless of water delivery volume, did not significantly increase the vegetative biomass of tomatoes (FIG. 35). Though not statistically significant, the application of Biofertilizer+D1 and Freeze Dry Formulation V1 tended to increase the total fruit yield per plant regardless of water delivery volume (FIG. 36A). Total fruit yield per plant tended to be greater than both the 50% GSP and 100% GSP treatments for all biofertilizer or freeze-dry treated plants. Only the application of Freeze Dry Formulation V1 in 40 ml of water meaningfully increased the mass of red fruit yield per plant over the 50% GSP and 100% GSP treatments (FIG. 36B). Additionally, the application of Biofertilizer+D1 in 20 ml of water and Freeze Dry Formulation V1 at both water delivery volumes tended to increase total fruit numbers over the 50% GSP and 100% GSP treatments (FIG. 36C). Average fruit size was consistent across all treatments with a slight trend towards larger mass fruits in the 50% GSP treatment (FIG. 36D). These results demonstrate similar efficacy between Biofertilizer+D1 standard liquid product and Freeze Dry Formulation V1 when applied to tomatoes.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A composition comprising i) a diluent and ii) a microorganism, wherein the diluent is derived from a plant, yeast, insect, crustacean or algae, and wherein the microorganism is a nitrogen fixing microorganism.

2. The composition of claim 1, wherein the diluent and/or the nitrogen fixing microorganism are in a dry formulation.

3. (canceled)

4. The composition of claim 1, wherein the diluent and/or the nitrogen fixing microorganism are in a liquid formulation.

5. (canceled)

6. The composition of claim 1, wherein the microorganism is a bacteria, wherein the bacteria is Xanthobacter autotrophicus.

7. The composition of claim 1, wherein the composition further comprises one, two, three, four, five, six, or more nitrogen fixing or non-nitrogen fixing microorganisms, wherein the nitrogen fixing or non-nitrogen fixing microorganisms are Xanthobacter autotrophicus, Pseudomonas fluorescens, Rhodopseudomonas palustris, Azospirillum lipoferum, Cupriavidus necator, or combinations thereof.

8.-12. (canceled)

13. The composition of claim 1, wherein the diluent is derived from a plant, wherein the plant is a coffee plant, a carrot, a potato, a citrus plant, a banana, an alfalfa grass, a tomato, a grape, a rice, or a maple tree.

14.-15. (canceled)

16. The composition of claim 1, wherein the diluent is derived from an algae, a yeast, or an insect.

17.-23. (canceled)

24. The composition of claim 1, wherein the nitrogen fixing microorganism is a bacteria, wherein the bacteria is Acidiphilium multivorum, Acidiphilium species, Alcaligenes paradoxus, Alcaligenes species, Arthrobacter species, Azoarcus indigens, Azohydromonas australica, Azohydromonas lata, Azohydromonas species, Azorhizobium caulinodans, Azospirillium brasiliense, Azospirillium spp., Azospirillum amazonsense, Azospirillum lipoferum, Azospirillum lipoferum (RSAL0111), Azospirillum species, Azospirillum thiophilum, Azotobacter chroococum (MCC 0055), Azotobacter spp., Azotobacter vinelandii, Azotobacter vinelandii (RSAV006), Bacillus megaterium, Bacillus pumilus, Bacillus amyloliquefaciens, Bacillus licheniformis, Bacillus subtilis, Beggiatoa alba, Beggiatoa species, Beijerinckia mobilis, Beijerinckia species, Bradyrhizobium elnakii, Bradyrhizobium japonicum, Bradyrhizobium japonicum (strain USDA 122), Bradyrhizobium species, Burkholderia species, Burkholderia vietnameiensis, Cupriavidus necator, Cupriavidus species, Cyanobacter species, Derxia gummosa, Derxia species, Gluconacetobacter diazotrophicus, Gluconacetobacter diazotrophicus (MCC 0046), Herbaspirillum autrotrophicum, Herbaspirillum frisingense (MCC 0052), Herbaspirillum species, Hydrogenophaga pseudoflava, Hydrogenophaga species, Klebsiella variicola, Kosakonia sacchari, Lactobacillus helveticus, Lactobacillus plantarum, Lactobacillus rhamnosus, Lactobacillus paracasei, Lactococcus lactis, Mesorhizobium alhagi, Mesorhizobium species, Methylibium petroleiphilum, Methylibium species, Methylocapsa aurea, Methylocapsa species, Methyloferula species, Methyloferula stellate, Methyloversatilis species, Methyloversatilis universalis, Microcyclus aquaticus, Microcyclus ebruneus, Microcyclus species, Nitrosococcus oceani, Nitrosococcus species, Nitrosomonas communis, Nitrospirillum amazonense, Nocardia autotrophica, Nocardia opaca, Nocardia species, Oligotropha carboxidovorans, Oligotropha species, Paenibacillus durus (MCC 0046), Pannonibacter phragmitetus, Pannonibacter species, Paracoccus denitrificans, Paracoccus pantrophus, Paracoccus species, Paracoccus yeei, Pelagibaca bermudensis, Pelagibaca species, Pseudomonas facilis, Pseudomonas fluorescens, Pseudomonas species, Pseudooceanicola atlanticus, Pseudooceanicola species, Ralstonia eutropha, Ralstonia species, Renobacter species, Renobacter vacuolatum, Rhizobium gallicum, Rhizobium japonicum, Rhizobium japonicum (MCC 0071), Rhizobium leguminosarum, Rhizobium leguminosarum biovar viciae, Rhizobium species, Rhizobium spp., Rhodobacter capsulatus, Rhodobacter species, Rhodobacter sphaeroides, Rhodomicrobium species, Rhodomicrobium vannielii, Rhodopseudomonas palustris, Rubrivivax gelatinosus, Rubrivivax species, Salipiger mucosus, Salipiger species, Sinorhizobium americanum, Sinorhizobium fredii, Sinorhizobium meliloti, Sinorhizobium species, Skermanella species, Skermanella stibiiresistens, Stappia aggregate, Stappia species, Thauera humireducens, Thauera species, Variovorax paradoxus, Variovorax species, Xanthobacter autotrophicus, Xanthobacter species, or combinations thereof.

25. The composition of claim 1, wherein the nitrogen fixing microorganism is a fungi, wherein the fungi is Glomus aggregatum, Glomus Intraradices, Glomus Mosseae, Glomus etunicatum, Trichoderma reesei, Candida utilis, Penicillium bilaiae, Saccharomyces cerevisiae, Trichoderma harzianum, Trichoderma virens, or combinations thereof.

26. (canceled)

27. The composition of claim 1, wherein the composition comprises (i) at least about 80% v/v, at least about 85% v/v, at least about 90% v/v, at least about 95% v/v, or at least about 99% v/v of the diluent; and/or (ii) at least about 1% v/v, at least about 5% v/v, at least about 10% v/v, at least about 15% v/v, or at least about 20% v/v of the microorganism.

28.-29. (canceled)

30. A kit comprising the composition of claim 1.

31.-32. (canceled)

33. A method of preparing a biofertilizer comprising:
i) obtaining an extract or slurry from a biomass to form a diluent, wherein the biomass is derived from plant, yeast, insect, crustacean or algae;
ii) combining the diluent with a nitrogen fixing microorganism to form a biofertilizer.

34.-35. (canceled)

36. A method of preparing a biofertilizer comprising:
i) adding a plant biomass or an algae biomass to a hot water bath to obtain a tea mixture;
ii) filtering the tea mixture and the plant biomass or an algae biomass to obtain a filtrate;
iii) centrifuging the filtrate to obtain a tea extract;
iv) filtering the tea extract to obtain a tea supernatant; and
v) combining the tea supernatant with a microorganism to form a biofertilizer.

37. A method of preparing a biofertilizer comprising:
a. providing a diluent in a dry formulation and a microorganism in a dry formulation, wherein the diluent is derived from a plant or algae, wherein the diluent has not been subjected to fermentation;
b. rehydrating the diluent in a dry formulation to form a rehydrated diluent;
c. rehydrating the microorganism in a dry formulation to form a rehydrated nitrogen fixing microorganism; and
d. combining the rehydrated diluent and the rehydrated nitrogen fixing microorganism to form a biofertilizer.

38. A method of increasing nitrogen utilization efficiency of a plant, comprising administering to the plant the composition of claim 1.

39. (canceled)

40. A method of increasing biomass, fruit quality, growth rate, lateral root density, or yield of a plant comprising administering to the plant the composition of claim 1.

41.-42. (canceled)

43. A method of increasing resistance to growth disease in a plant comprising administering to the plant the composition of claim 1.

44.-46. (canceled)

47. A method of increasing resistance to chemical over application in a plant comprising administering to the plant the composition of claim 1.

48. The method of claim 38, wherein the composition comprises (i) between about 1×10⁹ CFU/mL and about 4×10⁹ CFU/mL of the microbial cells; or (ii) about 5×10⁸ CFU/mL of a first microbial species, and about 5×10⁸ CFU/mL of a second microbial species.

49.-52. (canceled)

53. A method of improving the efficacy of a microorganism in a biofertilizer comprising contacting the microorganism with a diluent, wherein the diluent is derived from a plant, yeast, insect, or algae, wherein the microorganism is a nitrogen-fixing microorganism.

54.-63. (canceled)
